(19) United States
(12) Patent Application Publication
(10) Pub. No.: US 2024/0240206 A1
(43) Pub. Date: Jul. 18, 2024

(54) Insect cell production of parvoviral vectors with modified capsid proteins

(71) Applicant: uniQure biopharma B.V., Amsterdam (NL)

(72) Inventors: - Anggakusuma, Amsterdam (NL); Sebastian Niklas Kieper, Amsterdam (NL)

(73) Assignee: uniQure biopharma B.V., Amsterdam (NL)

(21) Appl. No.: 18/564,621

(22) PCT Filed: Jun. 2, 2022
(86) PCT No.: PCT/EP2022/065043

§ 371 Date: Nov. 28, 2023

(30) Foreign Application Priority Data
Jun. 2, 2021 (EP) 21177449.2

(51) Int. Cl. C12N 15/86; C07K 14/005; C12N 5/07

(52) U.S. Cl. CPC C12N 15/86; C07K 14/005; C12N 5/0601; C12N 2710/14043; C12N 2750/14152; C12N 2800/204

(57) ABSTRACT

The present invention relates to insect cells for producing parvoviral vectors with mosaic, chimeric and/or modified capsids. The insect cells of the invention comprise separate expression cassettes for the VP1 capsid protein and for the VP2 and VP3 proteins, which allow for the production of parvoviral vectors in which the VP1 capsid protein is of a different parvovirus or of a different serotype than the VP2 and VP3 protein and/or for the production of parvoviral vectors in which the VP1 capsid protein is modified, e.g. by the insertion of an exogenous amino acid sequence. Such exogenous amino acid sequence can e.g. encode a single domain antibody that targets the parvoviral vector to a specific tissue or type of cell. The invention further relates to method wherein the insect cells of the invention are used for the production of parvoviral vectors with mosaic, chimeric and/or modified capsids.

### FIELD OF THE INVENTION

The present invention relates to the fields of medicine, molecular virology, and gene therapy. The invention relates to means and methods for producing variants of parvoviral capsid proteins insect cells. In particular, the invention relates to the production of parvoviral vectors with modified capsid proteins that may be used in gene therapy.

### BACKGROUND OF THE INVENTION

The past decades have proven that parvoviruses such as Adeno-associated virus (AAV) can be successfully repurposed for gene therapy applications. The natural tropism of AAV serotypes allows for selecting suited serotypes depending on the application, e.g. whether specificity for a certain cell type or tissue is desired. However, these approaches can be limited by pre-existing neutralizing antibodies, by off-target specificity of the chosen serotype or by limitations imposed by tissues inaccessible to the AAV serotype (e.g. central nervous system due to the blood brain barrier).

One strategy to overcome these limitations has been to employ targeting technologies to redirect the AAV capsid to specific cell surface receptors (Foust et al., Nat. Biotechnol. 2009, 27: 59-65; Büning et al., Curr. Opin. Pharmacol. 2015, 24: 94-104), e.g., by genetic fusion or chemical ligation of the ligand for a cell surface receptor to a capsid protein (Ried et al., J. Virol. 2002, 76: 4559-4566; Ponnazhagan et al., J. Virol. 2002, 76, 12900-12907; Landegger et al., Nat. Biotechnol. 2017, 35: 280-284). The 3D structures of different AAV serotypes have provided a rational basis for eliminating binding to ubiquitously expressed receptors and for inserting a peptide or protein domain into an exposed loop of the viral capsid (Xie et al., Proc. Natl. Acad. Sci. USA, 2002, 99: 10405-10410; McCraw et al., Virology, 2012, 431: 40-49; Xie et al., Virology, 2011, 420: 10-19). For example, mutation of two arginine residues (R585, R588) to alanine abolishes binding of AAV2 to heparan sulfate proteoglycan (HSPG) (Kern et al., J. Virol. 2003, 77: 11072-11081; Boucas et al., J. Gene Med. 2009, 11: 1103-1113). Insertion of synthetic peptide libraries in this region followed by selection of specifically transduced cells or tissues has yielded AAV variants with improved target specificities (Büning et al., 2015, supra; Müller et al., Nat. Biotechnol. 2003, 21: 1040-1046; Maheshri et al., Nat. Biotechnol. 2006, 24: 198-204; Grimm et al., J. Virol. 2008, 82: 5887-5911; Deverman et al., Nat. Biotechnol. 2016, 34: 204-209; Körbelin et al., EMBO Mol. Med. 2016, 8: 609-625). More recently, Eichhoff et al. (Mol Ther Methods Clin Dev. 2019, 15: 211-220) reported that single chain antibodies (i.e. camelid VHHs) specific for cell-surface-proteins can be inserted into a surface loop of the VP1 capsid protein of AAV2 to redirect the cellular specificity of rAAV vectors with molecular precision.

However, the conventional approach for expressing such mosaic and/or modified AAV capsids relies on multi-plasmid expression platforms in mammalian cells (e.g. HEK293) that requires the simultaneous transfection with no less than 4 individual plasmids. The yield of intact and infectious AAV virions is strongly dependent on the transfection efficiency for each of those plasmids. For the production of research grade material and assessing the effect of the introduced modification, the yields of this approach suffices, nonetheless, scalability requires more robust and efficient production platforms.

It is therefore an object of the invention to provide means and methods for a robust, efficient and scalable insect cell-based production platform for recombinant parvoviral vectors with mosaic, chimeric or modified capsids.

### SUMMARY OF THE INVENTION

The present invention relates to an insect cell-based expression platform for the expression of mosaic, chimeric or modified parvoviral capsids.

In a first aspect, therefore, the invention relates to an insect cell comprising one or more nucleic acid constructs comprising: i) a first expression cassette comprising a first promoter operably linked to a nucleotide sequence encoding an mRNA, translation of which in the cell produces a parvoviral VP1 capsid protein; and, ii) a second expression cassette comprising a second promoter operably linked to a nucleotide sequence encoding an mRNA, translation of which in the cell produces parvoviral VP2 and VP3 capsid proteins.

In one embodiment, an insect cell of the invention comprises a first expression cassette wherein the nucleotide sequence encoding the mRNA for the parvoviral VP1 capsid protein, comprises at least one of: i) a suboptimal translation initiation codon for the VP1 coding sequence; ii) an inactivation of the native suboptimal translation initiation codon for the VP2 coding sequence; and, iii) an inactivation of the native ATG translation initiation codon for the VP3 coding sequence, and/or the nucleotide sequence encoding the mRNA for the parvoviral VP2 and VP3 capsid proteins, comprises at least one of: i) a deletion of the translation initiation codon for the VP1 coding sequence and optionally a deletion of at least a part of the VP1 coding sequence upstream of the VP2 initiation codon; ii) a suboptimal translation initiation codon for the VP2 coding sequence; and, iii) an ATG translation initiation codon for the VP3 coding sequence.

In one embodiment, an insect cell of the invention comprises a first expression cassette wherein the nucleotide sequence encoding the mRNA for the parvoviral VP1 capsid protein, comprises at least one of: i) the suboptimal translation initiation codon for the VP1 coding sequence is an ACG, CTG, TTG or GTG codon or an ATG codon in combination with an upstream out-of-frame initiation codon; ii) the native suboptimal translation initiation codon for the VP2 coding sequence is inactivated by replacement with another threonine codon; and, iii) the native ATG translation initiation codon for the VP3 coding sequence is inactivation by its deletion or by replacement with a codon coding for conservative substitution of methionine, preferably leucine.

In one embodiment, an insect cell of the invention comprises a first expression cassette wherein the nucleotide sequence encoding the mRNA for the parvoviral VP1 capsid protein encodes a common amino acid that has at least 90% amino acid sequence identity with a corresponding common amino acid encoded in the nucleotide sequence encoding the mRNA for the parvoviral VP2 and VP3 capsid proteins, and wherein the parts in the nucleotide sequences that encode the common amino acid sequences have less than 90% nucleotide sequence identity.

In one embodiment, there is provided for an insect cell, wherein the first and second expression cassettes are: a) both comprised in a single (episomal) nucleic acid construct, preferably a baculoviral vector; or, b) both comprised in at least one nucleic acid construct that is integrated in the genome of the insect cell, and wherein preferably, the first and second expression cassettes present in opposite directions of transcription.

In one embodiment, an insect cell of the invention is an insect cell wherein the first and second promoters are two different baculoviral promoters, preferably two different late or very late baculoviral promoters, more preferably two different baculoviral promoters selected from the group consisting of the polH, p10, p6.9 and pSel120 promoters, most preferably, the first promoter is the polH promoter and the second promoter is the p10 promoter.

In one embodiment, an insect cell of the invention is an insect cell wherein the parvoviral VP1 capsid protein is at least one of: a) a parvoviral VP1 capsid protein of a different parvovirus or of a different serotype than the parvoviral VP2 and VP3 capsid proteins; and, b) a parvoviral VP1 capsid protein comprising an insertion of an exogenous amino acid sequence. Preferably, the parvoviral VP1 capsid protein comprises an insertion of an exogenous amino acid sequence in an exposed loop of the capsid protein, wherein preferably, the exposed loop is at least one of the GH-L1 loop and the GH-L5 loop.

In one embodiment, an insect cell of the invention is an insect cell wherein the exogenous amino acid sequence encodes a single domain antibody, a ligand, designed ankyrin repeat protein (DARPin), an anticalin, an HDL-binding epitope or a reporter protein. Preferably, the single domain antibody, ligand, DARPin or anticalin has affinity for a cell surface marker that is specifically expressed on a target cell or target tissue, wherein preferably, the target cell or target tissue a central nervous system cell, a muscle cell, a liver cell, a synovial cell, a lymphocyte or a progenitor thereof, an endothelial cell, preferably a vascular endothelial cell, more preferably a vascular endothelial cell that is present in the blood brain barrier, or wherein the single domain antibody, ligand, DARPin or anticalin has affinity for HDL.

In one embodiment, an insect cell of the invention is an insect cell wherein: a) the parvoviral VP1 capsid protein is an AAV5 capsid protein; or, b) the parvoviral VP1 capsid protein is an AAV9 capsid protein and the parvoviral VP2 and VP3 capsid proteins are AAV5 capsid proteins.

In one embodiment, an insect cell of the invention is an insect cell further comprising at least one of: iii) a nucleic acid construct comprising at least one expression cassette for expression of nucleotide sequence encoding parvoviral Rep proteins; and, iv) a nucleic acid construct comprising a transgene that is flanked by at least one parvoviral inverted terminal repeat sequence, wherein preferably at least one of the nucleic acid construct in iii) and the nucleotide sequence in iv) is comprised in a baculoviral vector.

In another aspect, the invention pertains to a method for producing a recombinant parvoviral virion in a cell comprising the steps of: a) culturing an insect cell of the invention as herein defined under conditions such that recombinant parvoviral virion is produced; and, b) recovery of the recombinant parvoviral virion. Preferably, the recovery of the recombinant parvoviral virion in step b) comprises at least one of affinity-purification of the virion using an immobilised anti-parvoviral antibody, more preferably a single chain camelid antibody or a fragment thereof, or filtration over a filter having a nominal pore size of 30-70 nm.

In yet another aspect, the invention pertains to a kit of parts comprising at least an insect cell of the invention as herein defined and the nucleic acid construct and/or the nucleotide sequence as herein defined.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value.

As used herein, “an effective amount” is meant the amount of an agent required to ameliorate the symptoms of a disease relative to an untreated patient. The effective amount of active agent(s) used to practice the present invention for therapeutic treatment of, for example a cancer, varies depending upon the manner of administration, the age, body weight, and general health of the subject. Ultimately, the attending physician or veterinarian will decide the appropriate amount and dosage regimen. Such amount is referred to as an “effective” amount, which may be determined as genome copies per kilogram (GC/kg). Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the 5 product for use.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness 10 between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6× sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting expression of a gene in host cells or host organisms compatible with such sequences. These expression vectors typically include at least one “expression cassette” that is the functional unit capable of affecting expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in affecting expression may also be present, such as expression enhancer elements. The expression vector will be introduced into a suitable host cell and be able to affect expression of the coding sequence in an in vitro cell culture of the host cell. A preferred expression vector will be suitable for expression of viral proteins and/or nucleic acids, particularly recombinant parvoviral proteins and/or nucleic acids, such as baculoviral vectors for expression of parvoviral proteins and/or nucleic acids in insect cells.

A “parvoviral vector” is defined as a recombinantly produced parvovirus or parvoviral particle that comprises a polynucleotide to be delivered into a host cell, either in vivo, ex vivo or in vitro. An adeno-associated virus (AAV) vector is an example of a parvoviral vector. Herein, a parvoviral or AAV vector refers to the polynucleotide comprising part of the parvoviral genome, usually at least one ITR, and a transgene, which polynucleotide is preferably packaged in a parvoviral or AAV capsid.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which affect the translation, e.g., Kozak sequences, are known in insect cells. Expression control sequences can be of such nature as to modulate the nucleotide sequence to which it is operably linked such that lower expression levels or higher expression levels are achieved.

### DETAILED DESCRIPTION OF THE INVENTION

The present inventors have set out to develop an insect cell-based expression platform for the expression of mosaic, chimeric or modified parvoviral capsids. Its modularity allows for straightforward exchange of individual cap genes or for the modification of capsid proteins via peptide or even polypeptide insertions. While the production of unmodified parvoviral capsids in insect cells conventionally is accomplished by using three different (baculoviral) vectors, the production of capsid having e.g. a modified VP1 protein would require an additional fourth vector. This added complexity reduces the overall yield and robustness of the process. The requirement of an additional vector is circumvented by the inventors approach that employs two separate expression cassettes for the different capsid protein within one vector construct.

### The Insect Cell

In a first aspect, the invention provides an insect cell that comprises one or more nucleic acid constructs comprising a first expression cassette and a second expression cassette. The first expression cassette comprises a first promoter operably linked to a nucleotide sequence encoding an mRNA, translation of which in the cell produces a parvoviral VP1 capsid protein. In a preferred embodiment, the nucleotide sequence in the first expression cassette encodes an mRNA, translation of which in the cell produces only a parvoviral VP1 capsid protein (and not the VP2 and VP3 capsid proteins). The second expression cassette comprises a second promoter operably linked to a nucleotide sequence encoding an mRNA, translation of which in the cell produces parvoviral VP2 and VP3 capsid proteins.

In one embodiment, an insect cell of the invention comprises a) a first expression cassette wherein the nucleotide sequence encoding the mRNA for the parvoviral VP1 capsid protein, comprises at least one of: i) a suboptimal translation initiation codon for the VP1 coding sequence; ii) an inactivation of the native suboptimal translation initiation codon for the VP2 coding sequence; and, iii) an inactivation of the native ATG translation initiation codon for the VP3 coding sequence. In a preferred embodiment, the nucleotide sequence encoding the mRNA for the parvoviral VP1 capsid protein, comprises at least one of: ii) an inactivation of the native suboptimal translation initiation codon for the VP2 coding sequence; and, iii) an inactivation of the native ATG translation initiation codon for the VP3 coding sequence. More preferably, the nucleotide sequence encoding the mRNA for the parvoviral VP1 capsid protein, comprises ii) an inactivation of the native suboptimal translation initiation codon for the VP2 coding sequence; and, iii) an inactivation of the native ATG translation initiation codon for the VP3 coding sequence. In a most preferred embodiment, the nucleotide sequence encoding the mRNA for the parvoviral VP1 capsid protein, comprises: i) a suboptimal translation initiation codon for the VP1 coding sequence; ii) an inactivation of the native suboptimal translation initiation codon for the VP2 coding sequence; and, iii) an inactivation of the native ATG translation initiation codon for the VP3 coding sequence.

In one embodiment, an insect cell of the invention comprises a first expression cassette wherein the nucleotide sequence encoding the mRNA for the parvoviral VP1 capsid protein, comprises a suboptimal or non-canonical translation initiation codon selected from the group consisting of: ACG, ATT, ATA, AGA, AGG, AAA, CTG, CTT, CTC, CTA, CGA, CGC, TTG, TAG and GTG. In one embodiment, the suboptimal translation initiation codon is selected from the group consisting of GTG, CTG, ACG, and TTG, of which CTG is preferred. In another embodiment, the suboptimal translation initiation codon for the VP1 coding sequence consists of the combination of an ATG codon with an upstream out-of-frame initiation codon. In one embodiment, the ATG codon in combination with an upstream out-of-frame initiation codon as described in US-2020-0248206-A1, which is herein incorporated by reference. Thus, in one embodiment the upstream out-of-frame initiation codon can be selected from the group consisting of CTG, ATG, ACG, TTG, GTG, CTC and CTT. In one embodiment, the upstream out-of-frame initiation codon initiates an alternative open reading frame that encompasses the ATG translation initiation codon for the VP1 coding sequence, wherein preferably the alternative open reading frame encodes a peptide of up to 20 amino acids.

In one embodiment, an insect cell of the invention comprises a first expression cassette wherein the nucleotide sequence encoding the mRNA for the parvoviral VP1 capsid protein, comprises an inactivation of its native suboptimal (ACG) translation initiation codon for the VP2 coding sequence that is a replacement with another threonine codon. Thus, the native suboptimal ACG initiation codon for the VP2 coding sequence is replaced with one of ACT, ACC or ACA, of which ACA is preferred.

In one embodiment, an insect cell of the invention comprises a first expression cassette wherein the nucleotide sequence encoding the mRNA for the parvoviral VP1 capsid protein, comprises an inactivation of its native ATG translation initiation codon for the VP3 coding sequence that is either one of i) a deletion of the native ATG translation initiation codon for the VP3 coding sequence; or ii) a replacement of the native ATG translation initiation codon for the VP3 coding sequence with a codon coding for conservative amino acid substitution of methionine. Preferably, the native ATG translation initiation codon for the VP3 coding sequence is replaced with a codon coding for an aliphatic amino acid. More preferably, the native ATG translation initiation codon for the VP3 coding sequence is replaced with a codon coding for leucine, isoleucine or valine, of which leucine is most preferred.

In one embodiment, an insect cell of the invention comprises a second expression cassette wherein the nucleotide sequence encoding the mRNA for the parvoviral VP2 and VP3 capsid proteins comprises at least one of: i) a deletion of the translation initiation codon for the VP1 coding sequence and optionally a deletion of at least a part of the VP1 coding sequence upstream of the VP2 initiation codon; ii) a suboptimal translation initiation codon for the VP2 coding sequence; and, iii) an ATG translation initiation codon for the VP3 coding sequence. In a preferred embodiment, the nucleotide sequence encoding the mRNA for the parvoviral VP2 and VP3 capsid proteins comprises at least one of: ii) a suboptimal translation initiation codon for the VP2 coding sequence; and, iii) an ATG translation initiation codon for the VP3 coding sequence. In a more preferred embodiment, the nucleotide sequence encoding the mRNA for the parvoviral VP2 and VP3 capsid proteins comprises at least i) a deletion of the translation initiation codon for the VP1 coding sequence and preferably also a deletion of at least a part of the VP1 coding sequence upstream of the VP2 initiation codon. In a most preferred embodiment, the nucleotide sequence encoding the mRNA for the parvoviral VP2 and VP3 capsid proteins comprises: i) a deletion of the translation initiation codon for the VP1 coding sequence and optionally a deletion of at least a part of the VP1 coding sequence upstream of the VP2 initiation codon; ii) a suboptimal translation initiation codon for the VP2 coding sequence; and, iii) an ATG translation initiation codon for the VP3 coding sequence. In one embodiment, the nucleotide sequence encoding the mRNA for the parvoviral VP2 and VP3 capsid proteins comprises a suboptimal translation initiation codon for the VP2 coding sequence that is an ACG, CTG, TTG or GTG codon, of which ACG is most preferred.

As is known in the art, parvoviral VP1, VP2 and VP3 capsid proteins are naturally encoded by a single open reading frame. As a consequence the parvoviral capsid proteins comprise a common amino acid sequence consisting of the amino acid sequence of the VP3 protein that is also present in its entirety at the C-terminus of the VP1 and VP2 proteins, the latter of which is also present in its entirety at the C-terminus of the VP1 protein. As the insect cell of the invention comprises separate first and second expression cassettes for expression of respectively the VP1 and VP2/3 proteins, these expression cassettes, when using the native capsid coding sequences, will comprise identical nucleotide sequences that may cause instability of the expression cassettes in the insect cell due to homologous recombination between these identical sequences. To prevent such instability, therefore, in one embodiment of the cell of the invention, the nucleotide sequence (in the first expression cassette) encoding the mRNA, translation of which in the cell produces the parvoviral VP1 capsid protein encodes a common amino acid that has at least 90, 91, 92, 93, 94, 95, 96, 97, 98, 99 or 100% amino acid sequence identity with a corresponding common amino acid encoded in the nucleotide sequence (in the second expression cassette) encoding the mRNA, translation of which in the cell produces the parvoviral VP2 and VP3 capsid proteins, and wherein the parts in the nucleotide sequences that encode the common amino acid sequences have less than 90, 89, 88, 87, 86, 85, 84, 83, 82, 81, 80, 79, 78, 77, 76, 75, 74, 73, 72, 71, 70, 69, 68, 67, 66, 60% nucleotide sequence identity.

In a further embodiment, the nucleotide sequence encoding the common amino acid sequence of the mRNA, translation of which in the cell produces the parvoviral VP1 capsid protein has an improved codon usage bias for the insect cell as compared to the nucleotide sequence encoding the common amino acid sequence of the mRNA, translation of which in the cell produces the parvoviral VP2 and VP3 capsid proteins. Preferably, however, the nucleotide sequence encoding the common amino acid sequence of the mRNA, translation of which in the cell produces the parvoviral VP2 and VP3 capsid proteins has an improved codon usage bias for the insect cell as compared to the nucleotide sequence encoding the common amino acid sequences of the mRNA, translation of which in the cell produces the parvoviral VP1 capsid protein.

The adaptiveness of a nucleotide sequence encoding the common amino acid sequence to the codon usage of the host cell can be expressed as codon adaptation index (CAI). Preferably the codon usage is adapted to the insect cell wherein capsid proteins with the common amino acid sequence are expressed. Usually for baculoviral expression vectors (BEVs) this will be a cell of the genus Spodoptera, more preferably a Spodoptera frugiperda cell. The codon usage is thus preferably adapted to Spodoptera frugiperda or to an Autographa californica nucleopolyhedrovirus (AcMNPV) infected cell. A codon adaptation index is herein defined as a measurement of the relative adaptiveness of the codon usage of a gene towards the codon usage of highly expressed genes. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (Sharp and Li, 1987, Nucleic Acids Research 15: 1281-1295; also see: Kim et al., Gene. 1997, 25 199:293-301; zur Megede et al., Journal of Virology, 2000, 74: 2628-2635).

Preferably, the difference in codon adaptation index between the nucleotide sequences coding for the common amino acid sequences in the mRNA, translation of which in the cell produces the parvoviral VP2 and VP3 capsid proteins and the mRNA, translation of which in the cell produces the parvoviral VP1 capsid protein is at least 0.1, 0.2, 0.3, 0.4, 0.5, 0.6, 0.7, 0.8, 0.9 or 1.0 whereby more preferably, the CAI of the nucleotide sequence coding for the common amino acid sequence in the mRNA, translation of which in the cell produces the parvoviral VP2 and VP3 capsid proteins is at least 0.5, 0.6, 0.7, 0.8, 0.9 or 1.0.

In one embodiment, a nucleotide sequence coding for the mRNA, translation of which in the cell produces the VP2 and VP3 capsid proteins can be a wild type parvoviral nucleotide sequence, such as the nucleotide sequence of SEQ ID NO: 69 (coding for the AAV5 VP2 and VP3 capsid proteins), which is preferably used in combination with a nucleotide sequence coding for the mRNA, translation of which in the cell produces the AAV5 VP1 capsid protein that has been modified in the part coding for the common amino acid sequence it has in common with the VP2 and VP3 proteins to have less than 90, 89, 88, 87, 86, 85, 84, 83, 82, 81, 80, 79, 78, 77, 76, 75, 74, 73, 72, 71, 70, 69, 68, 67, 66, 60% nucleotide sequence identity, such as the nucleotide sequence of SEQ ID NO: 70 (coding for the AAV5 VP1 capsid protein), of which the part coding for the common amino acid sequence has no more than about 60% nucleotide sequence identity with SEQ ID NO: 69.

In one embodiment, an insect cell of the invention is an insect cell wherein the one or more nucleic acid constructs comprising the first and second expression cassettes of the invention are insect cell-compatible vectors. An “insect cell-compatible vector” is understood to be a nucleic acid molecule capable of productive transformation or transfection of an insect or insect cell. Exemplary insect cell-compatible vectors include plasmids, linear nucleic acid molecules, and recombinant viruses, such as baculoviruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In a preferred embodiment, the vector is a baculovirus, a viral vector, or a plasmid. In a more preferred embodiment, the vector is a baculovirus, i.e. the nucleic acid construct is a baculovirus-expression vector (BEV). It is well-known that baculovirus-expression vectors are particularly suitable for the transfer of nucleic acids to insect cells and methods for their use are described for example in: Summers and Smith, 1986, “A Manual of Methods for Baculovirus Vectors and Insect Culture Procedures”, Texas Agricultural Experimental Station Bull. No. 7555, College Station, Tex.; Luckow, 1991, In Prokop et al., “Cloning and Expression of Heterologous Genes in Insect Cells with Baculovirus Vectors' Recombinant DNA Technology and Applications”, 97-152; King and Possee, 1992, “The baculovirus expression system”, Chapman and Hall, United Kingdom; O'Reilly, Miller, and Luckow, 1992, “Baculovirus Expression Vectors: A Laboratory Manual”, New York; Freeman and Richardson, 1995, “Baculovirus Expression Protocols”, Methods in Molecular Biology, volume 39; U.S. Pat. No. 4,745,051; US2003148506; and WO 03/074714.

In one embodiment, an insect cell of the invention is an insect cell wherein the first and second expression cassettes are both comprised in a single nucleic acid construct. In one embodiment, the single nucleic acid construct is an episomal nucleic acid construct. In one embodiment, the single nucleic acid construct is a baculoviral vector. In one embodiment, the single nucleic acid construct comprises the first and second expression cassettes in opposite directions of transcription.

In one embodiment, an insect cell of the invention is an insect cell wherein the first and second expression cassettes are both comprised in at least one nucleic acid construct that is integrated in the genome of the insect cell. Thus, in one embodiment, the first and second expression cassettes are both comprised in a single nucleic acid construct that is integrated in the insect cell's genome. In another embodiment, the first and second expression cassettes are each comprised in a two separate nucleic acid constructs that are both integrated in the insect cell's genome. In one embodiment, the first and second expression cassettes are integrated in the insect cell's genome in opposite directions of transcription. Therefore, in one embodiment, the first and second expression cassettes are both integrated on the same chromosome in the insect cell. In one embodiment, the first and second expression cassettes are both integrated on the same chromosome in the insect cell within less than 0.5, 1.0, 2.0, 5.0, 10, 20, 50 or 100 kb from each other.

One of ordinary skill in the art knows how to stably introduce a nucleotide sequence into the insect genome and how to identify a cell having such a nucleotide sequence in the genome. The incorporation into the genome may be aided by, for example, the use of a vector comprising nucleotide sequences highly homologous to regions of the insect genome. The use of specific sequences, such as transposons, is another way to introduce a nucleotide sequence into a genome. The incorporation into the genome may be through one or more than one steps. Reference to the term “integrated” will be known to one in the art to also mean “stably integrated”.

An insect cell of the invention can be any cell that is suitable for the production of heterologous proteins. Preferably the insect cell allows for replication of baculoviral vectors and can be maintained in culture, more preferably in suspended culture. In a preferred embodiment, the insect cell allows for replication of recombinant parvoviral vectors, including rAAV vectors. For example, the cell line used can be from Spodoptera frugiperda, Drosophila, or mosquito, e.g., Aedes albopictus derived cell lines. Preferred insect cells or cell lines are cells from the insect species which are susceptible to baculovirus infection, including e.g. S2 (CRL-1963, ATCC), Se301, SeIZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA). A preferred insect cell according to the invention is an insect cell for production of recombinant parvoviral vectors.

Growing conditions for insect cells in culture, and production of heterologous products in insect cells in culture are well-known in the art and described e.g. in the above cited references on molecular engineering of insect cells (see also WO2007/046703).

In one embodiment, an insect cell of the invention is an insect cell wherein the first and second promoters are promoters that control expression of the operably linked nucleotide sequences in the insect cell. In one embodiment, the first and second promoters are baculoviral promoters, preferably late or very late baculoviral promoters. In one embodiment, the first and second promoters are two different promoters. In one embodiment, the first and second promoters are two different baculoviral promoters, preferably two different late or very late baculoviral promoters. In one embodiment, the first and second promoters are selected from the group consisting of the polH, p10, p6.9 and pSel120 promoters. In one embodiment, the first and second promoters are two different baculoviral promoters selected from the group consisting of the polH, p10, p6.9 and pSel120 promoters. In one embodiment, the first promoter is the polH promoter and the second promoter is the p10 promoter.

In one embodiment, an insect cell of the invention further comprises iii) a nucleic acid construct comprising at least one expression cassette for expression of parvoviral replicases or Rep proteins. Parvoviral, especially AAV, replicases are non-structural proteins encoded by the rep gene. In wild type parvoviruses the rep gene produces two overlapping messenger ribonucleic acids (mRNA) with different length, due to an internal P19 promoter. Each of these mRNA can be spliced out or not to eventually generate four Rep proteins, Rep78, Rep68, Rep52 and Rep40. The Rep78/68 and Rep52/40 are important for the ITR-dependent AAV genome or transgene replication and viral particle assembly. Rep78/68 serve as a viral replication initiator proteins and act as replicase for the viral genome (Chejanovsky and Carter, J Virol., 1990, 64:1764-1770; Hong et al., Proc Natl Acad Sci USA, 1992, 89:4673-4677; Ni., et al., J Virol., 1994, 68:1128-1138). The Rep52/40 protein is DNA helicase with 3′ to 5′ polarity and plays a critical role during packaging of viral DNA into empty capsids, where they are thought to be part of the packaging motor complex (Smith and Kotin, J. Virol., 1998, 4874-4881; King, et al., EMBO J., 2001, 20:3282-3291). To produce AAV from the baculoviral vectors in an insect cell platform, the presence of both Rep68 and Rep40 is not prerequisite (Urabe, et al., 2002).

A nucleotide sequence encoding a parvoviral Rep protein or encoding parvoviral Rep proteins, is herein understood as a nucleotide sequence encoding at least one of the two non-structural Rep proteins, Rep 78 and Rep52, that together are required and sufficient for parvoviral vector production in insect cells. The parvovirus nucleotide sequence preferably is from a dependovirus, more preferably from a human or simian adeno-associated virus (AAV) and most preferably from an AAV which normally infects humans (e.g., serotypes 1, 2, 3A, 3B, 4, 5, 6, 8 and 9) or primates (e.g., serotypes 1 and 4). Examples of nucleotide sequences encoding parvoviral Rep proteins are given in SEQ ID NO's: 60-66.

It is understood that the exact molecular weights of the Rep78 and Rep52 proteins, as well as the exact positions of the translation initiation codons may differ between different parvoviruses. However, the skilled person will know how to identify the corresponding position in nucleotide sequence from other parvoviruses than AAV-2. Preferably, the nucleotide sequence encodes parvovirus Rep proteins that are functionally active in the sense that they have the required activities of viral replication initiator protein, replicase of the viral genome, DNA helicase and packaging of viral DNA into empty capsids as described above, sufficient for parvoviral vector production in insect cells. In one embodiment, possible false translation initiation sites in the Rep protein coding sequences, other than the Rep78 and Rep52 translation initiation sites are eliminated. In one embodiment, putative splice sites that may be recognised in insect cells are eliminated from the Rep protein coding sequences. Elimination of these sites will be well understood by an artisan of skill in the art.

In one embodiment, the nucleic acid construct for expression of the parvoviral Rep proteins comprises a single expression cassette for expression of at least both the parvoviral Rep78 and Rep52 proteins. In one embodiment, the single expression cassette for expression of at least both the parvoviral Rep78 and Rep52 proteins comprises a single open reading frame encoding at least both the parvoviral Rep78 and Rep52 proteins and having a suboptimal translation initiation codon for the Rep78 coding sequence, which suboptimal initiation codon effect partial exon skipping so that both at least both the parvoviral Rep78 and Rep52 proteins are translated in the insect cell, as e.g. described in U.S. Pat. No. 8,512,981, incorporated herein by reference. Suitable suboptimal translation initiation codons include e.g. ACG, CTG, TTG and GTG. In another embodiment, the single expression cassette for expression of at least both the parvoviral Rep78 and Rep52 proteins comprises in 5′ to 3′order: (i) a first promoter linked operably to a 5′ portion of a first open reading frame of a parvovirus Rep78 protein, the first open reading frame comprising a translation initiation codon, (ii) an intron comprising a second insect cell promoter, the second promoter operably linked to a 5′ portion of an at least one additional open reading frame of a parvovirus Rep52 gene, wherein the at least one additional open reading frame comprises at least one additional translation initiation codon and overlaps with the 3′ portion of the first open reading frame, e.g. described in U.S. Pat. No. 8,945,918, incorporated herein by reference.

In another embodiment, the nucleic acid construct for expression of the parvoviral Rep proteins comprises at least two separate expression cassettes, one for expression of at least a parvoviral Rep78 protein and another for expression of at least a parvoviral Rep52 protein. Preferably, in this embodiment, the parvoviral Rep78 protein and the parvoviral Rep 52 protein comprise a common amino acid sequence comprising the amino acid sequence from the second amino acid to the most C-terminal amino acid of the parvoviral Rep 52 protein, wherein the common amino acid sequences of the parvoviral Rep78 protein and the parvoviral Rep52 protein are at least 90, 91, 92, 93, 94, 95, 96, 97, 98, 99 or 100% identical, and wherein the nucleotide sequence encoding the common amino acid sequence of the parvoviral Rep78 protein and the nucleotide sequence encoding the common amino acid sequences of the parvoviral Rep52 protein are less than 90, 89, 88, 87, 86, 85, 84, 83, 82, 81, 80, 79, 78, 77, 76, 75, 74, 73, 72, 71, 70, 69, 68, 67, 66, 60% identical, such as is described in U.S. Pat. No. 8,697,417, incorporated herein by reference. In a further embodiment, the nucleotide sequence encoding the common amino acid sequence of the parvoviral Rep78 protein has an improved codon usage bias for the cell as compared to the nucleotide sequence encoding the common amino acid sequences of the parvoviral Rep52 protein. Preferably, however, the nucleotide sequence encoding the common amino acid sequence of the parvoviral Rep52 protein has an improved codon usage bias for the cell as compared to the nucleotide sequence encoding the common amino acid sequences of the parvoviral Rep78 protein. Preferably, the difference in codon adaptation index (as defined hereinabove) between the nucleotide sequences coding for the common amino acid sequences in the parvoviral Rep78 protein and the parvoviral Rep52 protein is at least 0.1, 0.2, 0.3, 0.4, 0.5, 0.6, 0.7, 0.8, 0.9 or 1.0 whereby more preferably, the CAI of the nucleotide sequence coding for the common amino acid sequence in the parvoviral Rep52 protein is at least 0.5, 0.6, 0.7, 0.8, 0.9 or 1.0.

In one embodiment, the nucleotide sequences coding for the parvoviral Rep78 protein is SEQ ID NO: 66, coding for the wt AAV Rep78 protein and the nucleotide sequences coding for the parvoviral Rep52 selected from one of SEQ ID NO's: 61-64, each of which has been modified to have a different codon usage than the wild type Rep78 coding sequence of SEQ ID NO: 66. In a preferred embodiment, the nucleotide sequence coding for the parvoviral Rep78 protein is SEQ ID NO: 66, and is used in combination SEQ ID NO: 64 as nucleotide sequence coding for the parvoviral Rep52, the latter having been modified to differ as much as possible from SEQ ID NO: 66 in codon usage.

In one embodiment, the two separate expression cassettes for resp. the Rep78 and Rep52 proteins in the insect cell are optimised to obtain a desired molar ratio of the Rep78 to Rep52 proteins in the cell. Preferably, the combination of Rep78 and Rep52 expression cassettes in the cell produces a molar ratio of Rep78 to Rep52 in the range of 1:10 to 10:1, 1:5 to 5:1, or 1:3 to 3:1 in the (insect) cell. More preferably, the combination of Rep78 and Rep52 expression cassettes produces a molar ratio of Rep78 to Rep52 that is at least 1:2, 1:3, 1:5 or 1:10. The molar ratio of the Rep78 and Rep52 may be determined by means of Western blotting, preferably using a monoclonal antibody that recognizes a common epitope of both Rep78 and Rep52, or using e.g. a mouse anti-Rep antibody (303.9, Progen, Germany; dilution 1:50). A desired molar ratio of Rep78 to Rep52 can be obtained by the choice of the promoters in respectively the Rep78 and Rep52 expression cassettes as herein further described below. Alternatively or in combination, the desired molar ratio of Rep78 to Rep52 can be obtained by using means to reduce the steady state level of the at least one of parvoviral Rep 78 and 52 proteins. Thus, in one embodiment, the nucleotide sequence encoding the mRNA for the parvoviral Rep protein comprises a modification that affects a reduced steady state level of the parvoviral Rep protein. The reduced steady state condition can be achieved for example by truncating the regulation element or upstream promoter (Urabe et al., supra, Dong et al., supra), adding protein degradation signal peptide, such as the PEST or ubiquitination peptide sequence, substituting the start codon into a more suboptimal one, or by introduction of an artificial intron as described in WO 2008/024998. When using the two separate expression cassettes for resp. the Rep78 and Rep52 proteins in the insect cell the promoter in the Rep52 cassette is preferably stronger than the promoter in the Rep78 cassette. In one embodiment, the promoters in resp. the Rep78 and Rep52 cassettes are baculoviral promoters. In one embodiment, the promoters in resp. the Rep78 and Rep52 cassettes are distinct. In one embodiment, the Rep78 promoter is a delayed early baculoviral promoter, such as the 39 k promoter. In one embodiment, the Rep52 promoter is a late or very late baculovirus promoter, such as the polH, p10, p6.9 and pSel120 promoters. In one embodiment, the late or very late baculovirus promoter that is used in the Rep52 cassette is a different promoter than the first and second (late or very late baculovirus) promoters used in the first and second expression cassettes for expression of the capsid proteins.

In a preferred embodiment, the nucleotide sequence encoding at least one of parvoviral parvoviral Rep protein comprises an open reading frame that starts with a suboptimal translation initiation codon. The suboptimal initiation codon preferably is an initiation codon that affects partial exon skipping. Partial exon skipping is herein understood to mean that at least part of the ribosomes do not initiate translation at the suboptimal initiation codon of the Rep78 protein but may initiate at an initiation codon further downstream, whereby preferably the (first) initiation codon further downstream is the initiation codon of the Rep52 protein. Alternatively, the nucleotide sequence encoding a parvoviral Rep protein comprises an open reading frame that starts with a suboptimal translation initiation codon and has no initiation codons further downstream. The suboptimal initiation codon preferably affects partial exon skipping upon expression of the nucleotide sequence in an insect cell. Preferably, the suboptimal initiation codon affects partial exon skipping in an insect cell so as to produce in the insect cell a molar ratio of Rep78 to Rep52 in the range of 1:10 to 10:1, 1:5 to 5:1, or 1:3 to 3:1. The molar ratio of the Rep78 and Rep52 may be determined by means of Western blotting, preferably using a monoclonal antibody that recognizes a common epitope of both Rep78 and Rep52, or using e.g. a mouse anti-Rep antibody (303.9, Progen, Germany; dilution 1:50).

The term “suboptimal initiation codon” herein not only refers to the tri-nucleotide initiation codon itself but also to its context. Thus, a suboptimal initiation codon may consist of an “optimal” ATG codon in a suboptimal context, e.g. a non-Kozak context. However, more preferred are suboptimal initiation codons wherein the tri-nucleotide initiation codon itself is suboptimal, i.e. is not ATG. Suboptimal is herein understood to mean that the codon is less efficient in the initiation of translation in an otherwise identical context as compared to the normal ATG codon. Preferably, the efficiency of suboptimal codon is less than 90, 80, 60, 40 or 20% of the efficiency of the normal ATG codon in an otherwise identical context. Methods for comparing the relative efficiency of initiation of translation are known per se to the skilled person. Preferred suboptimal initiation codons may be selected from ACG, TTG, CTG, and GTG. More preferred is ACG. A nucleotide sequence encoding parvovirus Rep proteins, is herein understood as a nucleotide sequence encoding the non-structural Rep proteins that are required and sufficient for parvoviral vector production in insect cells such the Rep78 and Rep52 proteins.

In one embodiment, an insect cell of the invention further comprises iv) a nucleic acid construct comprising a transgene that is flanked by at least one parvoviral inverted terminal repeat (ITR) sequence.

In the context of the invention “at least one parvoviral inverted terminal repeat nucleotide sequence” is understood to mean a palindromic sequence, comprising mostly complementary, symmetrically arranged sequences also referred to as “A,” “B,” and “C” regions. The ITR functions as an origin of replication, a site having a “cis” role in replication, i.e. being a recognition site for trans acting replication proteins, such as e.g. Rep 78 (or Rep68), which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. A parvovirus replicating in a mammalian cell typically has two ITR sequences. It is, however, possible to engineer an ITR so that binding sites on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Preferably, however, two or another even number of regular ITRs are used. Most preferably, two ITR sequences are used. A preferred parvoviral ITR is an AAV ITR. More preferably AAV2 ITRs are used. For safety reasons it may be desirable to construct a recombinant parvoviral (rAAV) vector that is unable to further propagate after initial introduction into a cell in the presence of a second AAV. Such a safety mechanism for limiting undesirable vector propagation in a recipient may be provided by using rAAV with a chimeric ITR as described in US2003148506.

The term “flanked” with respect to a sequence that is flanked by another element(s) herein indicates the presence of one or more of the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence that is “flanked” by two other elements (e.g. ITRs), indicates that one element is located 5′ to the sequence and the other is located 3′ to the sequence; however, there may be intervening sequences there between. In a preferred embodiment a nucleotide sequence of (i) is flanked on either side by parvoviral inverted terminal repeat nucleotide sequences.

In the embodiments of the invention, the nucleotide sequence comprising the transgene (encoding either a gene product of interest, e.g. a protein, a nucleic acid molecule or a combination thereof, as further defined herein below) that is flanked by at least one parvoviral ITR sequence preferably becomes incorporated into the genome of a recombinant parvoviral (rAAV) vector produced in the insect cell. In one embodiment, the nucleotide sequence comprising the transgene is flanked by two parvoviral (AAV) ITR nucleotide sequences and wherein the transgene is located in between the two parvoviral (AAV) ITR nucleotide sequences. In one embodiment, the nucleotide sequence encoding a gene product of interest is incorporated into the recombinant parvoviral (rAAV) vector produced in the insect cell if it is located between two regular ITRs, or is located on either side of an ITR engineered with two D regions. Thus, in a preferred embodiment, the invention provides an insect cell, wherein the nucleotide sequence comprises two AAV ITR nucleotide sequences and wherein the at least one nucleotide sequence encoding a gene product of interest is located between the two AAV ITR nucleotide sequences.

Typically, the transgene, including ITRs and promoter & polyadenylation sequences, is 5,000 nucleotides (nt) or less in length. In another embodiment, an oversized DNA molecule, i.e. more than 5,000 nt in length, can be expressed in vitro or in vivo by using the AAV vector described by the present invention. An oversized DNA is here understood as a DNA exceeding the maximum AAV packaging limit of 5.5 kbp. Therefore, the generation of AAV vectors able to produce recombinant proteins that are usually encoded by larger genomes than 5.0 kb is also feasible.

AAV sequences that may be used in the present invention for the production of a recombinant AAV virion in insect cells can be derived from the genome of any AAV serotype. Generally, the AAV serotypes have genomic sequences of significant homology at the amino acid and the nucleic acid levels, provide an identical set of genetic functions, and produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). Any AAV serotype can be used as source of AAV nucleotide sequences for use in the context of the present invention. Preferably the AAV ITR sequences for use in the context of the present invention are derived from AAV1, AAV2, AAV4 and/or AAV7. Likewise, the Rep (Rep78/68 and Rep52/40) coding sequences are preferably derived from AAV1, AAV2, AAV4 and/or AAV7. The sequences coding for the VP1, VP2, and VP3 capsid proteins for use in the context of the present invention are defined in more details herein below.

AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82% (Bantel-Schaal et al., 1999, J. Virol., 73(2):939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells.

Modified “AAV” sequences also can be used in the context of the present invention, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8, AAV9, AAV10, AAV11, AAV12 or AAV13 ITR, Rep, or VP can be used in place of wild-type AAV ITR, Rep, or VP sequences.

In one embodiment of an insect cell of the invention, at least one of iii) the nucleic acid construct comprising at least one expression cassette for expression of the parvoviral Rep proteins; and iv) the nucleic acid construct comprising the transgene flanked by at least one parvoviral ITR; is comprised in an episomal nucleic acid construct, whereby preferably, the episomal nucleic acid construct is a baculoviral vector.

In one embodiment of an insect cell of the invention, both of iii) the nucleic acid construct comprising at least one expression cassette for expression of the parvoviral Rep proteins; and iv) the nucleic acid construct comprising the transgene flanked by at least one parvoviral ITR; are comprised in a single episomal nucleic acid construct, whereby preferably, the episomal nucleic acid construct is a baculoviral vector.

In one embodiment of an insect cell of the invention, iii) the nucleic acid construct comprising at least one expression cassette for expression of the parvoviral Rep proteins; and iv) the nucleic acid construct comprising the transgene flanked by at least one parvoviral ITR; are each comprised in a two separate episomal nucleic acid construct, whereby preferably, the episomal nucleic acid construct is a baculoviral vector.

In one embodiment, an insect cell of the invention is an insect cell wherein iii) the nucleic acid construct comprising at least one expression cassette for expression of the parvoviral Rep proteins is integrated into the genome of the insect cell. Preferably, when integrated into the insect cell's genome, the nucleic acid construct for expression of the parvoviral Rep proteins comprises at least two separate expression cassettes, one for expression of at least a parvoviral Rep78 protein and another for expression of at least a parvoviral Rep52 protein. Preferably, in this embodiment, the parvoviral Rep78 protein and the parvoviral Rep 52 protein comprise a common amino acid sequence comprising the amino acid sequence from the second amino acid to the most C-terminal amino acid of the parvoviral Rep 52 protein, wherein the common amino acid sequences of the parvoviral Rep78 protein and the parvoviral Rep52 protein are at least 90, 91, 92, 93, 94, 95, 96, 97, 98, 99 or 100% identical, and wherein the nucleotide sequence encoding the common amino acid sequence of the parvoviral Rep78 protein and the nucleotide sequence encoding the common amino acid sequences of the parvoviral Rep52 protein are less than 90, 89, 88, 87, 86, 85, 84, 83, 82, 81, 80, 79, 78, 77, 76, 75, 74, 73, 72, 71, 70, 69, 68, 67, 66, 60% identical, such as is described in U.S. Pat. No. 8,697,417, incorporated herein by reference.

In one embodiment, the two separate Rep78 and Rep52 expression cassettes are integrated in the insect cell's genome in opposite directions of transcription. Therefore, in one embodiment, the Rep78 and Rep52 expression cassettes are both integrated on the same chromosome in the insect cell. In one embodiment, the Rep78 and Rep52 expression cassettes are both integrated on the same chromosome in the insect cell within less than 0.5, 1.0, 2.0, 5.0, 10, 20, 50 or 100 kb from each other.

In one embodiment of the insect cell of the invention, the cell comprises tightly controlled inducible expression of Rep genes stably integrated in insect cell lines by providing means for reducing leaky expression under non-induced conditions while maintaining strong expression under induced conditions. Such insect cells are also referred to as iRep cells, or simply iRep and are described in more detail in co-pending application PCT/EP2021/058798, incorporated by reference herein. Thus, in this embodiment of the cell, the two separate Rep78 and Rep52 expression cassettes, e.g. as described above, are integrated in the insect cell's genome in opposite directions of transcription, whereby both expression cassettes comprise promoters that are operably linked to at least one enhancer element is dependent on a transcriptional transregulator, wherein introduction of the transcriptional transregulator into the insect cell induces transcription from the promoters in the Rep78 and Rep52 expression cassettes. In one embodiment, the promoters in the Rep78 and Rep52 expression cassettes are baculoviral promoters, the transcriptional transregulator is a baculoviral immediate-early protein (IE1) or its spice variant (IE0) and the transcriptional transregulator-dependent enhancer element is a baculoviral homologous region (hr) enhancer element, wherein preferably the baculovirus is Autographa californica multicapsid nucleopolyhedrovirus. In one embodiment, the hr enhancer element comprises at least one copy of the hr 28-mer sequence of SEQ ID NO: 67 and/or at least one copy of a of a sequence of which at least 20, 21, 22, 23, 24, 25, 26, or 27 nucleotides are identical to sequence SEQ ID NO: 67 and which binds to a baculoviral IE1 protein, and wherein the hr enhancer element, when operably linked to an expression cassette comprising a reporter gene operably linked to the polH promoter, a) under non-inducing conditions, the expression cassette with the hr enhancer element produces less reporter transcript than an otherwise identical expression cassette which comprises the hr2-0.9 element, or the cassette with the hr enhancer element produces less than a factor 1.1, 1.2, 1.5, 2, 5 or 10 of the amount reporter transcript produced by an otherwise identical expression cassette which comprises the hr4b element; and, b) under inducing conditions, the expression cassette with the hr enhancer element produces at least 50, 60, 70, 80, 90 or 100% of the amount of reporter transcript produced by an otherwise identical expression cassette which comprises the hr4b or the hr2-0.9 element. In one embodiment, the hr enhancer element is selected from the group consisting of hr1, hr2-0.9, hr3, hr4b and hr5, of which hr2-0.9, hr4b and hr5 are preferred, of which hr4b is most preferred.

### The Capsid Proteins and Modification Thereof

A nucleotide sequence encoding a parvoviral capsid (Cap) protein is herein understood to comprise nucleotide sequences encoding one or more of the three parvoviral capsid proteins, VP-1, -2 and -3. The parvovirus nucleotide sequence preferably is from a dependovirus, more preferably from a human or simian adeno-associated virus (AAV) and most preferably from an AAV which normally infects humans (e.g., serotypes 1, 2, 3A, 3B, 4, 5, 6, 7, 8, 9, 10, 11, 12 or 13) or primates (e.g., serotypes 1 and 4), of which the nucleotide and amino acid sequences are listed in Lubelski et al. US2017356008, which is incorporated herein in its entirety by reference. Thus, sequences coding for the VP1, and/or VP2 and VP3 capsid proteins for use in the context of the present invention can be taken from any of the known 42 serotypes, more preferably from AAV1, AAV2, AAV3, AAV4, AAV5, AAV6, AAV7, AAV8. AAV9, AAV10, AAV11, AAV12 or AAV13 or newly developed AAV-like particles obtained by e.g. capsid shuffling techniques and AAV capsid libraries, or from newly and synthetically designed, developed or evolved capsid, such as the Anc-80 capsid.

The sequences of the capsid proteins of the various serotypes are set out in the following:

| AAV serotype | VP1 | VP2 | VP3 |
|---|---|---|---|
| AAV1 | SEQ ID NO: 8 | SEQ ID NO: 9 | SEQ ID NO: 10 |
| AAV2 | SEQ ID NO: 11 | SEQ ID NO: 12 | SEQ ID NO: 13 |
| AAV3 | SEQ ID NO: 14 | SEQ ID NO: 15 | SEQ ID NO: 16 |
| AAV4 | SEQ ID NO: 17 | SEQ ID NO: 18 | SEQ ID NO: 19 |
| AAV5 | SEQ ID NO: 20 | SEQ ID NO: 21 | SEQ ID NO: 22 |
| AAV6 | SEQ ID NO: 23 | SEQ ID NO: 24 | SEQ ID NO: 25 |
| AAV7 | SEQ ID NO: 26 | SEQ ID NO: 27 | SEQ ID NO: 28 |
| AAV8 | SEQ ID NO: 29 | SEQ ID NO: 30 | SEQ ID NO: 31 |
| AAV9 | SEQ ID NO: 32 | SEQ ID NO: 33 | SEQ ID NO: 34 |
| AAV10 | SEQ ID NO: 35 | SEQ ID NO: 36 | SEQ ID NO: 37 |
| AAV11 | SEQ ID NO: 38 | SEQ ID NO: 39 | SEQ ID NO: 40 |
| AAV12 | SEQ ID NO: 41 | SEQ ID NO: 42 | SEQ ID NO: 43 |

In one embodiment, the one or more nucleic acid constructs comprising the first and second expression cassettes for expression of resp. the VP1 capsid protein and the parvoviral VP2 and VP3 capsid proteins encode for the wild type AAV VP1, and/or VP2 and VP3 capsid proteins having amino acid sequences as depicted in SEQ ID NO's: 8-43. Alternatively, the sequences can be man-made, for example, the sequence may be a hybrid form or may be codon optimized, such as for example by codon usage of AcmNPv or Spodoptera frugiperda. In one embodiment, the open reading frames for resp. the AAV VP1, and VP2 and 3 capsid proteins encode the AAV5 capsid proteins (SEQ ID NO's: 20, 21 and 22) or AAV2/5 hybrid capsid proteins, preferably (SEQ ID NO's: 44 and 45) or AAV8 capsid proteins (SEQ ID NO's: 29, 30 and 31). It is understood that the exact molecular weights of the capsid proteins, as well as the exact positions of the translation initiation codons may differ between different parvoviruses. However, the skilled person will know how to identify the corresponding position in nucleotide sequence from other parvoviruses than AAV5. Alternatively, the sequence encoding AAV capsid proteins is a man-made sequence, for example as a result of directed evolution experiments. This can include generation of capsid libraries via DNA shuffling, error prone PCR, bioinformatics rational design, site saturated mutagenesis. Resulting capsids are based on the existing serotypes but contain various amino acid or nucleotide changes that improve the features of such capsids. The resulting capsids can be a combination of various parts of existing serotypes, “shuffled capsids” or contain completely novel changes, i.e. additions, deletions or substitutions of one or more amino acids or nucleotides, organized in groups or spread over the whole length of gene or protein. See for example Schaffer and Maheshri; Proceedings of the 26th Annual International Conference of the IEEE EMBS San Francisco, CA, USA; Sep. 1-5, 2004, pages 3520-3523; Asuri et al., 2012, Molecular Therapy 20(2):329-3389; Lisowski et al., 2014, Nature 506(7488):382-386, herein incorporated by reference.

In one embodiment, the nucleotide sequence encoding the mRNA, translation of which in the cell produces (only) the parvoviral VP1 capsid protein comprises at least one modification of the nucleotide sequence encoding AAV VP1 capsid protein selected from among a G at nucleotide position 12, an A at nucleotide position 21, and a C at nucleotide position 24 of the VP1 open reading frame, wherein the nucleotide positions correspond to the nucleotide positions of the wild-type nucleotide sequences. A “potential/possible false start site” or “potential/possible false translation initiation codon” is herein understood to mean an in-frame ATG codon located in the coding sequence of the capsid protein(s). Elimination of possible false start sites for translation within the VP1 coding sequences of other serotypes will be well understood by an artisan of skill in the art, as will be the elimination of putative splice sites that may be recognized in insect cells. For example, the modification of the nucleotide at position 12 is not required for recombinant AAV5, since the nucleotide T is not giving rise to a false ATG codon. A further specific examples of a nucleotide sequence encoding parvovirus capsid proteins is given in SEQ ID NO: 46. Nucleotide sequences encoding parvoviral Cap of the invention may also be defined by their capability to hybridise with the nucleotide sequences of e.g. SEQ ID NO's: 44 and 46, respectively, under moderate, or preferably under stringent hybridisation conditions.

In one embodiment, the nucleotide sequences coding for resp. the VP1, and/or VP2 and VP3 capsid proteins as used in the invention comprise one or more modifications as described in WO2007/046703. Various further modifications of VP coding regions are known to the skilled artisan which could either increase yield of VP and virion or have other desired effects, such as altered tropism or reduce antigenicity of the virion. These modifications are within the scope of the present invention.

An important feature of the insect cell of the invention is that it allows for the production of the VP1 protein independently from the production of the VP2 and VP3 proteins. This provide the possibility to produce parvoviral virions of which VP1 protein is of a different serotype or type of dependovirus, than the different serotype or type of dependovirus of VP2 and VP3 proteins. Thus, in one embodiment, the invention relates to an insect cell wherein the nucleotide sequence in the first expression cassette, encoding (only) the parvoviral VP1 capsid protein, encodes a parvoviral VP1 capsid protein that is of a different serotype than the serotype of the parvoviral VP2 and VP3 capsid proteins, as encoded by the nucleotide sequence in the second expression cassette. In one embodiment, the parvoviral VP1 capsid protein is of a different AAV serotype than the AAV serotype of the parvoviral VP2 and VP3 capsid proteins. In one embodiment, the VP1 capsid protein is an AAV1 capsid protein and the VP2 and VP3 capsid proteins are AAV2 capsid proteins. In one embodiment, the VP1 capsid protein is an AAV1 capsid protein and the VP2 and VP3 capsid proteins are AAV3 capsid proteins. In one embodiment, the VP1 capsid protein is an AAV1 capsid protein and the VP2 and VP3 capsid proteins are AAV4 capsid proteins. In one embodiment, the VP1 capsid protein is an AAV1 capsid protein and the VP2 and VP3 capsid proteins are AAV5 capsid proteins. In one embodiment, the VP1 capsid protein is an AAV1 capsid protein and the VP2 and VP3 capsid proteins are AAV6 capsid proteins. In one embodiment, the VP1 capsid protein is an AAV1 capsid protein and the VP2 and VP3 capsid proteins are AAV7 capsid proteins. In one embodiment, the VP1 capsid protein is an AAV1 capsid protein and the VP2 and VP3 capsid proteins are AAV8 capsid proteins. In one embodiment, the VP1 capsid protein is an AAV1 capsid protein and the VP2 and VP3 capsid proteins are AAV9 capsid proteins. In one embodiment, the VP1 capsid protein is an AAV1 capsid protein and the VP2 and VP3 capsid proteins are AAV10 capsid proteins. In one embodiment, the VP1 capsid protein is an AAV1 capsid protein and the VP2 and VP3 capsid proteins are AAV11 capsid proteins. In one embodiment, the VP1 capsid protein is an AAV1 capsid protein and the VP2 and VP3 capsid proteins are AAV12 capsid proteins. In one embodiment, the VP1 capsid protein is an AAV1 capsid protein and the VP2 and VP3 capsid proteins are AAV13 capsid proteins. In one embodiment, the VP1 capsid protein is an AAV2 capsid protein and the VP2 and VP3 capsid proteins are AAV1 capsid proteins. In one embodiment, the VP1 capsid protein is an AAV2 capsid protein and the VP2 and VP3 capsid proteins are AAV3 capsid proteins. In one embodiment, the VP1 capsid protein is an AAV2 capsid protein and the VP2 and VP3 capsid proteins are AAV4 capsid proteins. In one embodiment, the VP1 capsid protein is an AAV2 capsid protein and the VP2 and VP3 capsid proteins are AAV5 capsid proteins. In one embodiment, the VP1 capsid protein is an AAV2 capsid protein and the VP2 and VP3 capsid proteins are AAV6 capsid proteins. In one embodiment, the VP1 capsid protein is an AAV2 capsid protein and the VP2 and VP3 capsid proteins are AAV7 capsid proteins. In one embodiment, the VP1 capsid protein is an AAV2 capsid protein and the VP2 and VP3 capsid proteins are AAV8 capsid proteins. In one embodiment, the VP1 capsid protein is an AAV2 capsid protein and the VP2 and VP3 capsid proteins are AAV9 capsid proteins. In one embodiment, the VP1 capsid protein is an AAV2 capsid protein and the VP2 and VP3 capsid proteins are AAV10 capsid proteins. In one embodiment, the VP1 capsid protein is an AAV2 capsid protein and the VP2 and VP3 capsid proteins are AAV11 capsid proteins. In one embodiment, the VP1 capsid protein is an AAV2 capsid protein and the VP2 and VP3 capsid proteins are AAV12 capsid proteins. In one embodiment, the VP1 capsid protein is an AAV2 capsid protein and the VP2 and VP3 capsid proteins are AAV13 capsid proteins. In one embodiment, the VP1 capsid protein is an AAV3 capsid protein and the VP2 and VP3 capsid proteins are AAV1 capsid proteins. In one embodiment, the VP1 capsid protein is an AAV3 capsid protein and the VP2 and VP3 capsid proteins are AAV2 capsid proteins. In one embodiment, the VP1 capsid protein is an AAV3 capsid protein and the VP2 and VP3 capsid proteins are AAV4 capsid proteins. In one embodiment, the VP1 capsid protein is an AAV3 capsid protein and the VP2 and VP3 capsid proteins are AAV5 capsid proteins. In one embodiment, the VP1 capsid protein is an AAV3 capsid protein and the VP2 and VP3 capsid proteins are AAV6 capsid proteins. In one embodiment, the VP1 capsid protein is an AAV3 capsid protein and the VP2 and VP3 capsid proteins are AAV7 capsid proteins. In one embodiment, the VP1 capsid protein is an AAV3 capsid protein and the VP2 and VP3 capsid proteins are AAV8 capsid proteins. In one embodiment, the VP1 capsid protein is an AAV3 capsid protein and the VP2 and VP3 capsid proteins are AAV9 capsid proteins. In one embodiment, the VP1 capsid protein is an AAV3 capsid protein and the VP2 and VP3 capsid proteins are AAV10 capsid proteins. In one embodiment, the VP1 capsid protein is an AAV3 capsid protein and the VP2 and VP3 capsid proteins are AAV11 capsid proteins. In one embodiment, the VP1 capsid protein is an AAV3 capsid protein and the VP2 and VP3 capsid proteins are AAV12 capsid proteins. In one embodiment, the VP1 capsid protein is an AAV3 capsid protein and the VP2 and VP3 capsid proteins are AAV13 capsid proteins. In one embodiment, the VP1 capsid protein is an AAV4 capsid protein and the VP2 and VP3 capsid proteins are AAV1 capsid proteins. In one embodiment, the VP1 capsid protein is an AAV4 capsid protein and the VP2 and VP3 capsid proteins are AAV2 capsid proteins. In one embodiment, the VP1 capsid protein is an AAV4 capsid protein and the VP2 and VP3 capsid proteins are AAV3 capsid proteins. In one embodiment, the VP1 capsid protein is an AAV4 capsid protein and the VP2 and VP3 capsid proteins are AAV5 capsid proteins. In one embodiment, the VP1 capsid protein is an AAV4 capsid protein and the VP2 and VP3 capsid proteins are AAV6 capsid proteins. In one embodiment, the VP1 capsid protein is an AAV4 capsid protein and the VP2 and VP3 capsid proteins are AAV7 capsid proteins. In one embodiment, the VP1 capsid protein is an AAV4 capsid protein and the VP2 and VP3 capsid proteins are AAV8 capsid proteins. In one embodiment, the VP1 capsid protein is an AAV4 capsid protein and the VP2 and VP3 capsid proteins are AAV9 capsid proteins. In one embodiment, the VP1 capsid protein is an AAV4 capsid protein and the VP2 and VP3 capsid proteins are AAV10 capsid proteins. In one embodiment, the VP1 capsid protein is an AAV4 capsid protein and the VP2 and VP3 capsid proteins are AAV11 capsid proteins. In one embodiment, the VP1 capsid protein is an AAV4 capsid protein and the VP2 and VP3 capsid proteins are AAV12 capsid proteins. In one embodiment, the VP1 capsid protein is an AAV4 capsid protein and the VP2 and VP3 capsid proteins are AAV13 capsid proteins. In one embodiment, the VP1 capsid protein is an AAV5 capsid protein and the VP2 and VP3 capsid proteins are AAV1 capsid proteins. In one embodiment, the VP1 capsid protein is an AAV5 capsid protein and the VP2 and VP3 capsid proteins are AAV2 capsid proteins. In one embodiment, the VP1 capsid protein is an AAV5 capsid protein and the VP2 and VP3 capsid proteins are AAV3 capsid proteins. In one embodiment, the VP1 capsid protein is an AAV5 capsid protein and the VP2 and VP3 capsid proteins are AAV4 capsid proteins. In one embodiment, the VP1 capsid protein is an AAV5 capsid protein and the VP2 and VP3 capsid proteins are AAV6 capsid proteins. In one embodiment, the VP1 capsid protein is an AAV5 capsid protein and the VP2 and VP3 capsid proteins are AAV7 capsid proteins. In one embodiment, the VP1 capsid protein is an AAV5 capsid protein and the VP2 and VP3 capsid proteins are AAV8 capsid proteins. In one embodiment, the VP1 capsid protein is an AAV5 capsid protein and the VP2 and VP3 capsid proteins are AAV9 capsid proteins. In one embodiment, the VP1 capsid protein is an AAV5 capsid protein and the VP2 and VP3 capsid proteins are AAV10 capsid proteins. In one embodiment, the VP1 capsid protein is an AAV5 capsid protein and the VP2 and VP3 capsid proteins are AAV11 capsid proteins. In one embodiment, the VP1 capsid protein is an AAV5 capsid protein and the VP2 and VP3 capsid proteins are AAV12 capsid proteins. In one embodiment, the VP1 capsid protein is an AAV5 capsid protein and the VP2 and VP3 capsid proteins are AAV13 capsid proteins. In one embodiment, the VP1 capsid protein is an AAV6 capsid protein and the VP2 and VP3 capsid proteins are AAV1 capsid proteins. In one embodiment, the VP1 capsid protein is an AAV6 capsid protein and the VP2 and VP3 capsid proteins are AAV2 capsid proteins. In one embodiment, the VP1 capsid protein is an AAV6 capsid protein and the VP2 and VP3 capsid proteins are AAV3 capsid proteins. In one embodiment, the VP1 capsid protein is an AAV6 capsid protein and the VP2 and VP3 capsid proteins are AAV4 capsid proteins. In one embodiment, the VP1 capsid protein is an AAV6 capsid protein and the VP2 and VP3 capsid proteins are AAV5 capsid proteins. In one embodiment, the VP1 capsid protein is an AAV6 capsid protein and the VP2 and VP3 capsid proteins are AAV7 capsid proteins. In one embodiment, the VP1 capsid protein is an AAV6 capsid protein and the VP2 and VP3 capsid proteins are AAV8 capsid proteins. In one embodiment, the VP1 capsid protein is an AAV6 capsid protein and the VP2 and VP3 capsid proteins are AAV9 capsid proteins. In one embodiment, the VP1 capsid protein is an AAV6 capsid protein and the VP2 and VP3 capsid proteins are AAV10 capsid proteins. In one embodiment, the VP1 capsid protein is an AAV6 capsid protein and the VP2 and VP3 capsid proteins are AAV11 capsid proteins. In one embodiment, the VP1 capsid protein is an AAV6 capsid protein and the VP2 and VP3 capsid proteins are AAV12 capsid proteins. In one embodiment, the VP1 capsid protein is an AAV6 capsid protein and the VP2 and VP3 capsid proteins are AAV13 capsid proteins. In one embodiment, the VP1 capsid protein is an AAV7 capsid protein and the VP2 and VP3 capsid proteins are AAV1 capsid proteins. In one embodiment, the VP1 capsid protein is an AAV7 capsid protein and the VP2 and VP3 capsid proteins are AAV2 capsid proteins. In one embodiment, the VP1 capsid protein is an AAV7 capsid protein and the VP2 and VP3 capsid proteins are AAV3 capsid proteins. In one embodiment, the VP1 capsid protein is an AAV7 capsid protein and the VP2 and VP3 capsid proteins are AAV4 capsid proteins. In one embodiment, the VP1 capsid protein is an AAV7 capsid protein and the VP2 and VP3 capsid proteins are AAV5 capsid proteins. In one embodiment, the VP1 capsid protein is an AAV7 capsid protein and the VP2 and VP3 capsid proteins are AAV6 capsid proteins. In one embodiment, the VP1 capsid protein is an AAV7 capsid protein and the VP2 and VP3 capsid proteins are AAV8 capsid proteins. In one embodiment, the VP1 capsid protein is an AAV7 capsid protein and the VP2 and VP3 capsid proteins are AAV9 capsid proteins. In one embodiment, the VP1 capsid protein is an AAV7 capsid protein and the VP2 and VP3 capsid proteins are AAV10 capsid proteins. In one embodiment, the VP1 capsid protein is an AAV7 capsid protein and the VP2 and VP3 capsid proteins are AAV11 capsid proteins. In one embodiment, the VP1 capsid protein is an AAV7 capsid protein and the VP2 and VP3 capsid proteins are AAV12 capsid proteins. In one embodiment, the VP1 capsid protein is an AAV7 capsid protein and the VP2 and VP3 capsid proteins are AAV13 capsid proteins. In one embodiment, the VP1 capsid protein is an AAV8 capsid protein and the VP2 and VP3 capsid proteins are AAV1 capsid proteins. In one embodiment, the VP1 capsid protein is an AAV8 capsid protein and the VP2 and VP3 capsid proteins are AAV2 capsid proteins. In one embodiment, the VP1 capsid protein is an AAV8 capsid protein and the VP2 and VP3 capsid proteins are AAV3 capsid proteins. In one embodiment, the VP1 capsid protein is an AAV8 capsid protein and the VP2 and VP3 capsid proteins are AAV4 capsid proteins. In one embodiment, the VP1 capsid protein is an AAV8 capsid protein and the VP2 and VP3 capsid proteins are AAV5 capsid proteins. In one embodiment, the VP1 capsid protein is an AAV8 capsid protein and the VP2 and VP3 capsid proteins are AAV6 capsid proteins. In one embodiment, the VP1 capsid protein is an AAV8 capsid protein and the VP2 and VP3 capsid proteins are AAV7 capsid proteins. In one embodiment, the VP1 capsid protein is an AAV8 capsid protein and the VP2 and VP3 capsid proteins are AAV9 capsid proteins. In one embodiment, the VP1 capsid protein is an AAV8 capsid protein and the VP2 and VP3 capsid proteins are AAV10 capsid proteins. In one embodiment, the VP1 capsid protein is an AAV8 capsid protein and the VP2 and VP3 capsid proteins are AAV11 capsid proteins. In one embodiment, the VP1 capsid protein is an AAV8 capsid protein and the VP2 and VP3 capsid proteins are AAV12 capsid proteins. In one embodiment, the VP1 capsid protein is an AAV8 capsid protein and the VP2 and VP3 capsid proteins are AAV13 capsid proteins. In one embodiment, the VP1 capsid protein is an AAV9 capsid protein and the VP2 and VP3 capsid proteins are AAV1 capsid proteins. In one embodiment, the VP1 capsid protein is an AAV9 capsid protein and the VP2 and VP3 capsid proteins are AAV2 capsid proteins. In one embodiment, the VP1 capsid protein is an AAV9 capsid protein and the VP2 and VP3 capsid proteins are AAV3 capsid proteins. In one embodiment, the VP1 capsid protein is an AAV9 capsid protein and the VP2 and VP3 capsid proteins are AAV4 capsid proteins. In one embodiment, the VP1 capsid protein is an AAV9 capsid protein and the VP2 and VP3 capsid proteins are AAV5 capsid proteins. In one embodiment, the VP1 capsid protein is an AAV9 capsid protein and the VP2 and VP3 capsid proteins are AAV6 capsid proteins. In one embodiment, the VP1 capsid protein is an AAV9 capsid protein and the VP2 and VP3 capsid proteins are AAV7 capsid proteins. In one embodiment, the VP1 capsid protein is an AAV9 capsid protein and the VP2 and VP3 capsid proteins are AAV8 capsid proteins. In one embodiment, the VP1 capsid protein is an AAV9 capsid protein and the VP2 and VP3 capsid proteins are AAV10 capsid proteins. In one embodiment, the VP1 capsid protein is an AAV9 capsid protein and the VP2 and VP3 capsid proteins are AAV11 capsid proteins. In one embodiment, the VP1 capsid protein is an AAV9 capsid protein and the VP2 and VP3 capsid proteins are AAV12 capsid proteins. In one embodiment, the VP1 capsid protein is an AAV9 capsid protein and the VP2 and VP3 capsid proteins are AAV13 capsid proteins. In one embodiment, the VP1 capsid protein is an AAV10 capsid protein and the VP2 and VP3 capsid proteins are AAV1 capsid proteins. In one embodiment, the VP1 capsid protein is an AAV10 capsid protein and the VP2 and VP3 capsid proteins are AAV2 capsid proteins. In one embodiment, the VP1 capsid protein is an AAV10 capsid protein and the VP2 and VP3 capsid proteins are AAV3 capsid proteins. In one embodiment, the VP1 capsid protein is an AAV10 capsid protein and the VP2 and VP3 capsid proteins are AAV4 capsid proteins. In one embodiment, the VP1 capsid protein is an AAV10 capsid protein and the VP2 and VP3 capsid proteins are AAV5 capsid proteins. In one embodiment, the VP1 capsid protein is an AAV10 capsid protein and the VP2 and VP3 capsid proteins are AAV6 capsid proteins. In one embodiment, the VP1 capsid protein is an AAV10 capsid protein and the VP2 and VP3 capsid proteins are AAV7 capsid proteins. In one embodiment, the VP1 capsid protein is an AAV10 capsid protein and the VP2 and VP3 capsid proteins are AAV8 capsid proteins. In one embodiment, the VP1 capsid protein is an AAV10 capsid protein and the VP2 and VP3 capsid proteins are AAV9 capsid proteins. In one embodiment, the VP1 capsid protein is an AAV10 capsid protein and the VP2 and VP3 capsid proteins are AAV11 capsid proteins. In one embodiment, the VP1 capsid protein is an AAV10 capsid protein and the VP2 and VP3 capsid proteins are AAV12 capsid proteins. In one embodiment, the VP1 capsid protein is an AAV10 capsid protein and the VP2 and VP3 capsid proteins are AAV13 capsid proteins. In one embodiment, the VP1 capsid protein is an AAV11 capsid protein and the VP2 and VP3 capsid proteins are AAV1 capsid proteins. In one embodiment, the VP1 capsid protein is an AAV11 capsid protein and the VP2 and VP3 capsid proteins are AAV2 capsid proteins. In one embodiment, the VP1 capsid protein is an AAV11 capsid protein and the VP2 and VP3 capsid proteins are AAV3 capsid proteins. In one embodiment, the VP1 capsid protein is an AAV11 capsid protein and the VP2 and VP3 capsid proteins are AAV4 capsid proteins. In one embodiment, the VP1 capsid protein is an AAV11 capsid protein and the VP2 and VP3 capsid proteins are AAV5 capsid proteins. In one embodiment, the VP1 capsid protein is an AAV11 capsid protein and the VP2 and VP3 capsid proteins are AAV6 capsid proteins. In one embodiment, the VP1 capsid protein is an AAV11 capsid protein and the VP2 and VP3 capsid proteins are AAV7 capsid proteins. In one embodiment, the VP1 capsid protein is an AAV11 capsid protein and the VP2 and VP3 capsid proteins are AAV8 capsid proteins. In one embodiment, the VP1 capsid protein is an AAV11 capsid protein and the VP2 and VP3 capsid proteins are AAV9 capsid proteins. In one embodiment, the VP1 capsid protein is an AAV11 capsid protein and the VP2 and VP3 capsid proteins are AAV10 capsid proteins. In one embodiment, the VP1 capsid protein is an AAV11 capsid protein and the VP2 and VP3 capsid proteins are AAV12 capsid proteins. In one embodiment, the VP1 capsid protein is an AAV11 capsid protein and the VP2 and VP3 capsid proteins are AAV13 capsid proteins. In one embodiment, the VP1 capsid protein is an AAV12 capsid protein and the VP2 and VP3 capsid proteins are AAV1 capsid proteins. In one embodiment, the VP1 capsid protein is an AAV12 capsid protein and the VP2 and VP3 capsid proteins are AAV2 capsid proteins. In one embodiment, the VP1 capsid protein is an AAV12 capsid protein and the VP2 and VP3 capsid proteins are AAV3 capsid proteins. In one embodiment, the VP1 capsid protein is an AAV12 capsid protein and the VP2 and VP3 capsid proteins are AAV4 capsid proteins. In one embodiment, the VP1 capsid protein is an AAV12 capsid protein and the VP2 and VP3 capsid proteins are AAV5 capsid proteins. In one embodiment, the VP1 capsid protein is an AAV12 capsid protein and the VP2 and VP3 capsid proteins are AAV6 capsid proteins. In one embodiment, the VP1 capsid protein is an AAV12 capsid protein and the VP2 and VP3 capsid proteins are AAV7 capsid proteins. In one embodiment, the VP1 capsid protein is an AAV12 capsid protein and the VP2 and VP3 capsid proteins are AAV8 capsid proteins. In one embodiment, the VP1 capsid protein is an AAV12 capsid protein and the VP2 and VP3 capsid proteins are AAV9 capsid proteins. In one embodiment, the VP1 capsid protein is an AAV12 capsid protein and the VP2 and VP3 capsid proteins are AAV10 capsid proteins. In one embodiment, the VP1 capsid protein is an AAV12 capsid protein and the VP2 and VP3 capsid proteins are AAV11 capsid proteins. In one embodiment, the VP1 capsid protein is an AAV12 capsid protein and the VP2 and VP3 capsid proteins are AAV13 capsid proteins. In one embodiment, the VP1 capsid protein is an AAV13 capsid protein and the VP2 and VP3 capsid proteins are AAV1 capsid proteins. In one embodiment, the VP1 capsid protein is an AAV13 capsid protein and the VP2 and VP3 capsid proteins are AAV2 capsid proteins. In one embodiment, the VP1 capsid protein is an AAV13 capsid protein and the VP2 and VP3 capsid proteins are AAV3 capsid proteins. In one embodiment, the VP1 capsid protein is an AAV13 capsid protein and the VP2 and VP3 capsid proteins are AAV4 capsid proteins. In one embodiment, the VP1 capsid protein is an AAV13 capsid protein and the VP2 and VP3 capsid proteins are AAV5 capsid proteins. In one embodiment, the VP1 capsid protein is an AAV13 capsid protein and the VP2 and VP3 capsid proteins are AAV6 capsid proteins. In one embodiment, the VP1 capsid protein is an AAV13 capsid protein and the VP2 and VP3 capsid proteins are AAV7 capsid proteins. In one embodiment, the VP1 capsid protein is an AAV13 capsid protein and the VP2 and VP3 capsid proteins are AAV8 capsid proteins. In one embodiment, the VP1 capsid protein is an AAV13 capsid protein and the VP2 and VP3 capsid proteins are AAV9 capsid proteins. In one embodiment, the VP1 capsid protein is an AAV13 capsid protein and the VP2 and VP3 capsid proteins are AAV10 capsid proteins. In one embodiment, the VP1 capsid protein is an AAV13 capsid protein and the VP2 and VP3 capsid proteins are AAV11 capsid proteins. In one embodiment, the VP1 capsid protein is an AAV13 capsid protein and the VP2 and VP3 capsid proteins are AAV12 capsid proteins. In a preferred embodiment, the VP1 capsid protein is an AAV9 capsid protein and the VP2 and VP3 capsid proteins are AAV5 capsid proteins in order to combine AAV9's ability to cross the blood-brain barrier with AAV5 ability to escape neutralising antibodies.

In one embodiment, the invention relates to an insect cell wherein the nucleotide sequence in the first expression cassette, encoding (only) the parvoviral VP1 capsid protein, encodes a parvoviral VP1 capsid protein comprising an insertion of an exogenous amino acid sequence.

In principle the invention allows for the production of parvoviral vectors with an insertion of an exogenous amino acid sequence in all three of VP1, VP2 and VP3 capsid proteins. However, insertion in all three parvoviral capsid proteins can interfere with capsid assembly and/or other viral functions, such as e.g. infectivity, due to steric hindrance by the inserted exogenous amino acid sequence, particularly in the case of larger exogenous amino acid sequence (e.g. >50 or 100 amino acids). It is therefore preferred that the exogenous amino acid sequence is inserted only in a VP1 capsid protein. During assembly, VP1, VP2, and VP3 are incorporated at a ratio of resp. 1:1:10 into parvoviral capsids. Insertion of the exogenous amino acid sequence into only a VP1 capsid protein therefore does not interfere with capsid assembly or infectivity.

In one embodiment, an exogenous amino acid sequence is inserted into an exposed loop of a parvoviral capsid protein. Preferably, the exogenous amino acid sequence is inserted into an exposed loop of the parvoviral VP1 capsid protein, more preferably, the exogenous amino acid sequence is inserted into an exposed loop of only the parvoviral VP1 capsid protein.

X-ray crystallographic studies have shown that the icosahedral 3-fold axis of a parvoviral capsid, such as that of AAV5, is surrounded by protrusions to which two finger-like VP loops contribute (Govindasamy et al., J Virol. 2013, 87(20):11187-99. doi: 10.1128/JVI.00867-13; Zhang et al., Nat Commun. 2019, 10(1):3760. doi: 10.1038/s41467-019-11668-x). These two finger-like VP loops, termed GH-L1 and GH-L5, form the two outmost protrusions of the capsid exterior (FIG. 5A). Residues located within GH-L1 and GH-L5 (variable regions (VR) IV and VIII respectively) were shown to be involved in receptor attachment and transduction. It has however been demonstrated that the GH-L1 and GH-L5 loops can accommodate insertions without compromising infectivity of the resulting virions (Judd et al., Mol Ther Nucleic Acids. 2012; 1: e54). In one embodiment therefore, the exogenous amino acid sequence is inserted into at least one of the GH-L1 loop and the GH-L5 loop of a parvoviral capsid protein, of which the GH-L1 loop is preferred. Preferably, the exogenous amino acid sequence is inserted into at least one of the GH-L1 loop and the GH-L5 loop of the parvoviral VP1 capsid protein, whereby the GH-L1 loop is preferred. More preferably, the exogenous amino acid sequence is inserted into at least one of the GH-L1 loop and the GH-L5 loop of only the parvoviral VP1 capsid protein, whereby the GH-L1 loop is preferred (see Judd et al., 2012, supra).

In one embodiment, an exogenous amino acid sequence is inserted in the GH-L1 loop in the variable region (VR) IV, which, with reference to the amino acid sequence of the AAV5 VP1 capsid protein (SEQ ID NO: 20), comprises amino acid positions F438-F449 (corresponding to amino acid positions L445-F462 in VP1 of AAV2). In one embodiment, an exogenous amino acid sequence is inserted in the GH-L1 loop between T444 and G445, between G445 and G446, or between G446 and V447, of which G446 and V447 are preferred.

In one embodiment, an exogenous amino acid sequence is inserted in the GH-L5 loop in the VR VIII, which, with reference to the amino acid sequence of the AAV5 VP1 capsid protein (SEQ ID NO: 20), comprises amino acid positions Q574-P580 (corresponding to amino acid positions Q584-A590 in VP1 of AAV2). In one embodiment, an exogenous amino acid sequence is inserted in the GH-L5 loop between Q574 and S575, between S575 and S576, between S576 and T577, between T577 and T578, between T578 and A579, or between A579 and P580.

Corresponding positions for the insertion of an exogenous amino acid sequence in the GH-L1 or GH-L5 loops, resp. VR IV or VR VIII, of VP1 proteins of other AAV serotypes, or of other parvoviruses can be identified by alignment of the VP1 amino acid sequences with that of the AAV5 VP1 (see e.g. FIG. 5B), e.g. using an alignment algorithm and settings as herein described above.

It is further understood that the insertion of exogenous amino acid sequence as described herein above can be an insertion in the strict sense, i.e. without the removal of any native amino acid residues from the amino acid sequence of the capsid protein. However, also include in the invention is the insertion of an exogenous amino acid sequence with the concomitant replacement of 1, 2, 3, 4, 5, 6, 7, 8, 9, 10, 11, 12, 13, 14, 15, 16, 17, 18, 19, 20, 22, 25, or 30 native amino acid residues from the amino acid sequence of the capsid protein, whereby the native amino acid residues are preferably replace with the exogenous amino acid sequence.

In one embodiment, an exogenous amino acid sequence that is inserted in a parvoviral capsid protein comprises a linker sequence on at least one of the N-terminal or C-terminal end of the amino acid sequence as it is inserted. In one embodiment, the linker sequence is a flexible linker sequence. Suitable flexible linker-amino acid sequences are known in the art (e.g. from Chen et al., 2013, Adv Drug Deliv Rev. 65(10): 1357-1369). Flexible linkers are usually applied when the joined domains require a certain degree of movement or interaction. They are generally composed of small, non-polar (e.g. Gly) or polar (e.g. Ser or Thr) amino acids. The small size of these amino acids provides flexibility, and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. Preferred flexible linkers have sequences consisting primarily of stretches of Gly and Ser residues (“GS” linker). An example of preferred (and widely used) flexible linker has the sequence of (Gly-Gly-Gly-Gly-Ser)n. By adjusting the copy number “n”, the length of this GS linker can be optimized to achieve appropriate separation of the functional domains, or to maintain necessary inter-domain interactions. In one embodiment, n is 1, 2, 3, 4, or 5. Besides the GS linkers, many other flexible linkers have been designed for recombinant fusion proteins. These flexible linkers are also rich in small or polar amino acids such as Gly and Ser, but can contain additional amino acids such as Thr and Ala to maintain flexibility, as well as polar amino acids such as Lys and Glu to improve solubility, such as e.g. the flexible linkers having the amino acid sequences of SEQ ID NO's: 47 and 48, that have been applied for the construction of a bioactive scFv's.

In one embodiment, the exogenous amino acid sequence that is inserted in a parvoviral capsid protein to be expressed in an insect cell of the invention can in principle be any amino acid sequence. The inserted exogenous amino acid sequence can have any length. The inserted exogenous amino acid sequence can be a relatively short amino acid sequence of e.g. 3, 4, 5, 6, 7, 8, 9, 10, 11, 12, 13, 14, 15, 16, 17, 18, 19, 20, 22, 24, 26, 28 or 30 amino acids, e.g. comprising an epitope, or the inserted exogenous amino acid sequence can be a longer amino acid sequence comprising e.g. 40, 50, 60, 70, 80, 90, 100, 120, 150, 200, 300, 400 or more amino acid, e.g. comprising a functional domain such as a variable domain of an antibody, or a domain with enzymatic activity.

In one embodiment, the exogenous amino acid sequence that is inserted in a parvoviral capsid protein to be expressed in an insect cell of the invention comprises and/or encodes a single chain antibody or single domain antibody. A single-domain antibody is an antibody fragment consisting of a single monomeric variable antibody domain. Like a whole antibody, it is able to bind selectively to a specific antigen. With a molecular weight of only 12-15 kDa, single-domain antibodies are much smaller than conventional antibodies (150-160 kDa) which are composed of two heavy protein chains and two light chains. Single-domain antibodies can be engineered from heavy-chain antibodies found in camelids; these are called VHH fragments (Harmsen and De Haard 2007, Appl. Microbiol. Biotechnol. 77: 13-22). Cartilaginous fishes also have heavy-chain antibodies (IgNAR, ‘immunoglobulin new antigen receptor’), from which single-domain antibodies called VNAR fragments can be obtained (English et al., 2020, Antibody Ther. 3: 1-9). Single-domain antibody can be obtained by immunization of dromedaries, camels, llamas, alpacas or sharks with the desired antigen and subsequent isolation of the mRNA coding for heavy-chain antibodies by methods well-known in the art. By using a library construction methods, e.g. based on PCR-extension assembly and self-ligation (EASeL), phage displayed antibody libraries can be established. Screening techniques like phage display and ribosome display help to identify the clones binding the antigen (Feng et al., 2019, Antibody Ther. 2: 1-11; Arbabi Ghahroudi et al., 1997, FEBS Letters. 414: 521-6).

In one embodiment, the exogenous amino acid sequence that is inserted in a parvoviral capsid protein to be expressed in an insect cell of the invention comprises and/or encodes a ligand. The ligand can e.g. be a ligand (with affinity) for a receptor expressed at a cell surface of a particular cell.

In one embodiment, the exogenous amino acid sequence that is inserted in a parvoviral capsid protein to be expressed in an insect cell of the invention comprises and/or encodes a designed ankyrin repeat protein (DARPin) (see e.g. Pluckthun, 2015, Annu. Rev. Pharmacol. Toxicol. 55 (1): 489-511) or an anticalin (Skerra, 2001, Rev. Mol. Biol. 74, 257-275).

In one embodiment, a single domain antibody, ligand, anticalin or DARPin as defined hereinabove, has affinity for a cell surface marker. The term “cell surface marker” refers to a protein or a carbohydrate structure that is present on the surface of a target cell. In one embodiment, the cell surface marker is membrane protein, i.e. a protein that is attached to and/or integrated into the membrane of a cell, and of which at least a part is exposed on the outside of the target cell, such that it is available for binding by the single domain antibody, ligand, anticalin or DARPin. In one embodiment, the cell surface marker is, the carbohydrate structure is part of a glycolipid or glycoprotein that is expressed on the surface of the target cell. The membrane protein or the glycoprotein can e.g. be multi-span homo trimer membrane protein, a type-II single span membrane protein, a GPI-anchor membrane protein, an integrin, a type I membrane protein (e.g. members of the immunoglobulin superfamily), a receptor, such as a cytokine, growth factor or hormone receptor, an Fc receptor, a Toll-like receptors, C-type lectin-like receptors or a G-protein coupled receptor.

In one embodiment, the single domain antibody, ligand, anticalin or DARPin has affinity for a cell surface marker that is specifically expressed on a target cell or target tissue. In one embodiment, the target cell is a central nervous system (CNS) cell, e.g. at least one of a neuron, an oligodendrocyte, an astrocyte and a microglial cell. In one embodiment, the target cell is a muscle cell, e.g. at least one of a myocyte and a myotube. In one embodiment, the target cell is a liver cell such as an hepatocyte. In one embodiment, the target cell is a synovial cell. In one embodiment, the target cell is a lymphocyte, such as a B cell or a T cell or a progenitor thereof. In one embodiment, the target cell is an epithelial cell. In one embodiment, the target cell is an endothelial cell, preferably a vascular endothelial cell, more preferably a vascular endothelial cell that is present in the blood brain barrier (BBB). In one embodiment, the target cell is a tumor cell. In one embodiment, the single domain antibody, ligand, anticalin or DARPin has affinity for a cell surface marker that is selected from the group consisting of: an EGF receptor, an FGF receptor, CD71, TMEM30A, CD11b, HER2, a purinoceptor, an asialoglycoprotein receptor (ASGPR), CD200, POPDC2, NTCP, ZNT8 and VCAM1.

In one embodiment, a single domain antibody, ligand, anticalin or DARPin as defined hereinabove, has affinity for high-density lipoprotein (HDL). In one embodiment, the single domain antibody, ligand, anticalin or DARPin has affinity for Apolipoprotein A-I (ApoA-1).

In one embodiment, the exogenous amino acid sequence that is inserted in a parvoviral capsid protein to be expressed in an insect cell of the invention comprises and/or encodes an HDL-binding epitope. In one embodiment, the HDL-binding epitope is an ApoA-1-binding epitope. In one embodiment, the ApoA-1-binding epitope is derived from an ApoA-1-binding protein selected from the group consisting of: PON1 P1, PON1 P2, LCAT s108, LCAT K249, ABCA1, r587, q597, C1477 surround, S1506 surround and apoB. In one embodiment, wherein the ApoA-1-binding epitope comprises an amino acid sequence with no more than 1 or 2 amino acid differences from an amino acid sequence selected from the group consisting of SEQ ID NO's: 49-59. Parvoviral vectors that have affinity for and bind to HDL and ApoA1 improve the spread of the vector in the liver.

In one embodiment, the exogenous amino acid sequence that is inserted in a parvoviral capsid protein to be expressed in an insect cell of the invention comprises and/or encodes a reporter protein. In one embodiment, the reporter protein is luciferase or a fluorescent protein such as GFP, EGFP, mCherry, mOrange, Cerulean, mTurquoise2, Citrine or TagBFP.

### Parvoviral Vectors

The present invention relates to insect cells for producing recombinant parvoviruses, in particular dependoviruses such as infectious human or simian AAV, and the components thereof (e.g., a parvovirus genome) for use as vectors for introduction and/or expression of nucleic acids in mammalian cells, preferably human cells. In particular, the invention relates to means and methods that allow for the production in insect cells of such parvoviral vectors with modifications in one or more of their capsid proteins.

A “parvoviral vector” is defined as a recombinantly produced parvovirus or parvoviral particle that comprises a polynucleotide to be delivered into a host cell, either in vivo, ex vivo or in vitro. Examples of parvoviral vectors include e.g., adeno-associated virus vectors. Herein, a parvoviral vector construct refers to the polynucleotide comprising the viral genome or part thereof, and a transgene. Viruses of the Parvoviridae family are small DNA viruses. The family Parvoviridae may be divided between two subfamilies: the Parvovirinae, which infect vertebrates, and the Densovirinae, which infect invertebrates, including insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 1, 2, 3A, 3B, 4, 5, 6, 7, 8, 9, 10, 11, 12 and 13) or primates (e.g., serotypes 1 and 4), and related viruses that infect other warm-blooded animals (e.g., bovine, canine, equine, and ovine adeno-associated viruses). Further information on parvoviruses and other members of the Parvoviridae is described in Kenneth I. Berns, “Parvoviridae: The Viruses and Their Replication,” Chapter 69 in Fields Virology (3d Ed. 1996). For convenience, the present invention is further exemplified and described herein by reference to AAV. It is however understood that the invention is not limited to AAV but may equally be applied to other parvoviruses.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural viral particle (VP) proteins. The VP proteins (VP1, -2 and -3) form the capsid. The terminal 145 nt ITRs are self-complementary and are organized so that an energetically stable intramolecular duplex forming a T-shaped hairpin may be formed. These hairpin structures function as an origin for viral DNA replication, serving as primers for the cellular DNA polymerase complex. Following wildtype (wt) AAV infection in mammalian cells the Rep genes (i.e. Rep78 and Rep52) are expressed from the P5 promoter and the P19 promoter, respectively, and both Rep proteins have a function in the replication and packaging of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production. The three capsid proteins, VP1, VP2 and VP3 are expressed from a single VP reading frame from the p40 promoter. wtAAV infection in mammalian cells relies for the capsid proteins production on a combination of alternate usage of two splice acceptor sites and the suboptimal utilization of an ACG initiation codon for VP2.

A “recombinant parvoviral or AAV vector” (or “rAAV vector”) herein refers to a vector comprising one or more polynucleotide sequences of interest, genes of interest or “transgenes” that is/are flanked by at least one parvoviral or AAV inverted terminal repeat sequence (ITR). Preferably, the transgene(s) is/are flanked by ITRs, one on each side of the transgene(s). Such rAAV vectors can be replicated and packaged into infectious viral particles when present in an insect host cell that is expressing AAV rep and cap gene products (i.e. AAV Rep and Cap proteins). When an rAAV vector is incorporated into a larger nucleic acid construct (e.g. in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions.

### The Transgene

The nucleotide sequence comprising the transgene as defined herein above may thus comprise a nucleotide sequence encoding a gene product of interest (for expression in the mammalian cell) or encoding a nucleotide sequence targeting a gene of interest (for silencing said gene of interest in a mammalian cell), and may be located such that it will be incorporated into an recombinant parvoviral (rAAV) vector replicated in the insect cell. In the context of the invention it is understood that a particularly preferred mammalian cell in which the “gene product of interest” is to be expressed or silenced, is a human cell. Any nucleotide sequence can be incorporated for later expression in a mammalian cell transfected with the recombinant parvoviral (rAAV) vector produced in accordance with the present invention. The nucleotide sequence may e.g. encode a protein or it may express an RNAi agent, i.e. an RNA molecule that is capable of RNA interference such as, e.g. an shRNA (short hairpinRNA) or an siRNA (short interfering RNA). “siRNA” means a small interfering RNA that is a short-length double-stranded RNA that are not toxic in mammalian cells (Elbashir et al., 2001, Nature 411: 494-98; Caplen et al., 2001, Proc. Natl. Acad. Sci. USA 98: 9742-47). In a preferred embodiment, the nucleotide sequence comprising the transgene may comprise two coding nucleotide sequences, each encoding one gene product of interest for expression in a mammalian cell. Each of the two nucleotide sequences encoding a product of interest is located such that it will be incorporated into a recombinant parvoviral (rAAV) vector replicated in the insect cell.

The product of interest for expression in a mammalian cell may be a therapeutic gene product. A therapeutic gene product can be a polypeptide, or an RNA molecule (si/sh/miRNA), or other gene product that, when expressed in a target cell, provides a desired therapeutic effect. A desired therapeutic effect can for example be the ablation of an undesired activity (e.g. VEGF), the complementation of a genetic defect, the silencing of genes that cause disease, the restoration of a deficiency in an enzymatic activity or any other disease-modifying effect. Examples of therapeutic polypeptide gene products include, but are not limited to growth factors, factors that form part of the coagulation cascade, enzymes, lipoproteins, cytokines, neurotrophic factors, hormones and therapeutic immunoglobulins and variants thereof. Examples of therapeutic RNA molecule products include miRNAs effective in silencing diseases, including but not limited to polyglutamine diseases, dyslipidaemia or amyotrophic lateral sclerosis (ALS).

The diseases that can be treated using a recombinant parvoviral (rAAV) vector produced in accordance with the present invention are not particularly limited, other than generally having a genetic cause or basis. For example, the disease that may be treated with the disclosed vectors may include, but are not limited to, acute intermittent porphyria (AIP), age-related macular degeneration, Alzheimer's disease, arthritis, Batten disease, Canavan disease, Citrullinemia type 1, Crigler Najjar, congestive heart failure, cystic fibrosis, Duchene muscular dystrophy, dyslipidemia, glycogen storage disease type I (GSD-I), hemophilia A, hemophilia B, hereditary emphysema, homozygous familial hypercholesterolemia (HoFH), Huntington's disease (HD), Leber's congenital amaurosis, methylmalonic academia, ornithine transcarbamylase deficiency (OTC), Parkinson's disease, phenylketonuria (PKU), spinal muscular atrophy, paralysis, Wilson disease, epilepsy, Pompe disease, amyotrophic lateral sclerosis (ALS), Tay-Sachs disease, hyperoxaluria 9PH-1), spinocerebellar ataxia type 1 (SCA-1), SCA-3, u-dystrophin, Gaucher's types II or III, arrhythmogenic right ventricular cardiomyopathy (ARVC), Fabry disease, familial Mediterranean fever (FMF), proprionic acidemia, fragile X syndrome, Rett syndrome, Niemann-Pick disease and Krabbe disease. Examples of therapeutic gene products to be expressed include N-acetylglucosaminidase, alpha (NaGLU), Treg167, Treg289, EPO, IGF, IFN, GDNF, FOXP3, Factor VIII, Factor IX and insulin.

Alternatively, or in addition as another gene product, the nucleotide sequence comprising the transgene as defined herein above may further comprise a nucleotide sequence encoding a polypeptide that serves as a selection marker protein to assess cell transformation and expression. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel, supra. Furthermore, the nucleotide sequence comprising the transgene as defined herein above may comprise a further nucleotide sequence encoding a polypeptide that may serve as a fail-safe mechanism that allows to cure a subject from cells transduced with the recombinant parvoviral (rAAV) vector of the invention, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the transgenic cells in the subject (see e.g. Clair et al., 1987, Antimicrob. Agents Chemother. 31: 844-849).

The nucleotide sequence comprising a transgene as defined herein above for expression in a mammalian cell, further preferably comprises at least one mammalian cell-compatible expression control sequence, e.g. a promoter, that is/are operably linked to the sequence coding for the gene product of interest. Many such promoters are known in the art (see Sambrook and Russel, 2001, supra). Constitutive promoters that are broadly expressed in many cell-types, such as the CMV promoter may be used. However, more preferred will be promoters that are inducible, tissue-specific, cell-type-specific, or cell cycle-specific. For example, for liver-specific expression (as disclosed in PCT/EP2019/081743) a promoter may be selected from an a1-anti-trypsin promoter, a thyroid hormone-binding globulin promoter, an albumin promoter, LPS (thyroxine-binding globin) promoter, HCR-ApoCII hybrid promoter, HCR-hAAT hybrid promoter and an apolipoprotein E promoter, LP1, HLP, minimal TTR promoter, FVIII promoter, hyperon enhancer, ealb-hAAT. Other examples include the E2F promoter for tumor-selective, and, in particular, neurological cell tumor-selective expression (Parr et al., 1997, Nat. Med. 3:1145-9) or the IL-2 promoter for use in mononuclear blood cells (Hagenbaugh et al., 1997, J Exp Med; 185: 2101-10).

Various modifications of the nucleotide sequences as defined above, including e.g. the wild-type parvoviral sequences, for proper expression in insect cells is achieved by application of well-known genetic engineering techniques such as described e.g. in Sambrook and Russell (2001) “Molecular Cloning: A Laboratory Manual (3rd edition), Cold Spring Harbor Laboratory, Cold Spring Harbor Laboratory Press, New York. Various further modifications of coding regions are known to the skilled artisan which could increase yield of the encode proteins. These modifications are within the scope of the present invention.

### Method for Producing a Recombinant Parvoviral Virion

In a further aspect, the invention provides for a method for producing a recombinant parvoviral virion. The method preferably comprises the steps of: a) culturing an insect cell as defined herein; b) providing the cell cultured in a) with the nucleotide sequences as defined herein; and, c) recovery of the recombinant parvoviral virion. In one embodiment, the cell culture in a) is transfected, also known as infected, with the nucleotide sequences as defined herein.

Recovery preferably comprises the step of affinity-purification of the (virions comprising the) recombinant parvoviral (rAAV) vector using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, 2001, Biotechnol. 74: 277-302). The antibody for affinity-purification of rAAV preferably is an antibody that specifically binds an epitope on an AAV capsid protein, whereby preferably the epitope is an epitope that is present on capsid protein of more than one AAV serotype. E.g. the antibody may be raised or selected on the basis of specific binding to AAV2 capsid but at the same time also it may also specifically bind to AAV1, AAV3 and AAV5 capsids.

In a further embodiment, wherein recovery of the recombinant parvoviral virion in step c) comprises at least one of affinity-purification of the virion using an immobilised anti-parvoviral antibody, preferably a single chain camelid antibody or a fragment thereof, and filtration over a filter having a nominal pore size of 30-70 nm.

Therefore, in one embodiment the invention provides a method for producing a recombinant parvoviral virion in a cell. The method preferably comprising the steps of: a) culturing an insect cell as defined herein; b) infecting the cell cultured in a) with the nucleotide sequences as defined herein; and, c) recovery of the recombinant parvoviral virion wherein recovery of the recombinant parvoviral virion in step b) comprises at least one of affinity-purification of the virion using an immobilised anti-parvoviral antibody, preferably a single chain camelid antibody or a fragment thereof, or filtration over a filter having a nominal pore size of 30-70 nm.

In a further aspect the invention relates to a batch of parvoviral virions produced in the above described methods of the invention. A “batch of parvoviral virions” is herein defined as all parvoviral virions that are produced in the same round of production, optionally per container of insect cells.

In yet a further aspect, the invention relates to a pharmaceutical composition comprising parvoviral virions, e.g. AAV vectors, produced in the above described methods of the invention, and at least one pharmaceutically acceptable carrier.

### Kits

In a further aspect, the invention provides for a kit of parts comprising at least an insect cell as defined herein and a baculoviral vector and/or the nucleotide sequences as defined herein.

The present invention has been described above with reference to a number of exemplary embodiments as shown in the drawings. Modifications and alternative implementations of some parts or elements are possible, and are included in the scope of protection as defined in the appended claims.

### DESCRIPTION OF THE FIGURES

FIG. 1. Schematic overview of possible BacCapCap designs. Each expression cassette is driven by its own promoter, the late baculovirus promoters p10 and polH. Promoter orientation may be changed depending on desired properties of the expression cassettes. This arrangement potentially opens up the possibility of having temporal control over protein expression, e.g. by choosing early and late promoters. The ORFs for the polH-driven cassette is located on the plus-strand (5′-3′ orientation) while the p10 cassette is located on the minus-strand (3′-5′ orientation). The modularity of two separate expression cassettes allows for independent expression of VP2 and VP3 as well as either (A) modified VP1 (e.g. large peptide insertions such as single domain antibody (sdAb) sequences inserted between VP1 amino acids 444-445/445-446) or (B) VP1 from a different serotype. In case both cassettes contain coding sequences from the same serotype or sequences with a high level of homology, codon shuffling can be employed to reduce the occurrence of homologous recombination between the two cassettes.

FIG. 2. (A) Constructs used in transient transfection assay for Western Blot analysis and batch-binding purification. The VP123 construct (Cap) contains the baculovirus enhancer sequence as well as the AAV5 cap gene driven by the polH promoter. The VHH-VP1-VP23 construct (CapCap) contains the AAV5 VP2-VP3 ORF under control of the p10 promoter and the VHH-VP1 ORF under control of the polH promoter. Both expression cassettes are preceded by the baculovirus enhancer sequence required for baculovirus transactivation. (B) Western Blot of the 6-well plate transient transfection assay. The Cap construct expresses the unmodified VP1, VP2 and VP3 proteins. In contrast, the CapCap construct expressed the modified, higher molecular weight VHH-VP1 protein next to unmodified VP2 and VP3. Star symbol in VHH-VP1+VP23 indicates western blot background band (as found in negative control, also seen in FIG. 4). (C) SDS-PAGE of Batch-binding purification of 125 ml shaker flask transient transfection assay. The SDS PAGE analysis is representative of capsid protein stoichiometry of assembled virions purified by using the AVB Sepharose affinity resin from crude lysate. The VP123 construct shows the bona fide AAV stoichiometry of 1:1:10 (VP1:VP2:VP3). In contrast, AAV particles resulting from the CapCap construct display strong expression of VP2 and VP3 but an underrepresentation of VHH-VP1.

FIG. 3. (A) Schematic overview of BacCapCap designs recombined into BEV expression system. Each expression cassette is driven by its own promoter, the late baculovirus promoters p10 and polH. Two Designs were tested in which the interspacing between the promoters was varied (Design A: 20 bp interspacing; Design B: 141 bp interspacing). The ORFs for the polH-driven cassette is located on the plus-strand (5′-3′ orientation) while the p10 cassette is located on the minus-strand (3′-5′ orientation). The p10 promoter drives expression of unmodified VP2 and VP3, the polH promoter drives expression of a chimeric VP1 protein composed of AAV2 and AAV5 elements and containing a large peptide insertion (161 amino acids). In order to avoid homology between the ORFs the modified-VP1 was codon shuffled with respect to the VP2-VP3 cassette. (B) Western-Blot detection (using α-VP3 primary antibody) of modified VP1 and unmodified VP2 and VP3 proteins in cleared lysates of Sf+ insect cells transduced with CapCap-containing Baculovirus. Two representative clones are shown. (C) SDS-PAGE analysis of AVB-Sepharose purified rAAV expressed from CapCap Design A and B. rAAV was purified from cleared lysates used for WesternBlots shown in (B). (D) Genome copy quantification of AVB-Sepharose purified rAAV by taqMan quantitative PCR using primer-probe combination specific for SEAP-transgene CMV promoter. (E) Potency of purified rAAV measured as SEAP-activity in culture medium of Huh7 cells transduced at MOI of 10⁵.

FIG. 4. Western blot image of 6-well transient transfection assay. Cells were transfected with GFP expressing plasmid as a Western Blot negative control, a plasmid containing the VP123 wild-type cassette, a plasmid only containing VP1 or VP23 and the CapCap VHH-VP1+VP23 cassette. Protein expression was induced by transactivation using BacTrans (+) or not induced (−).

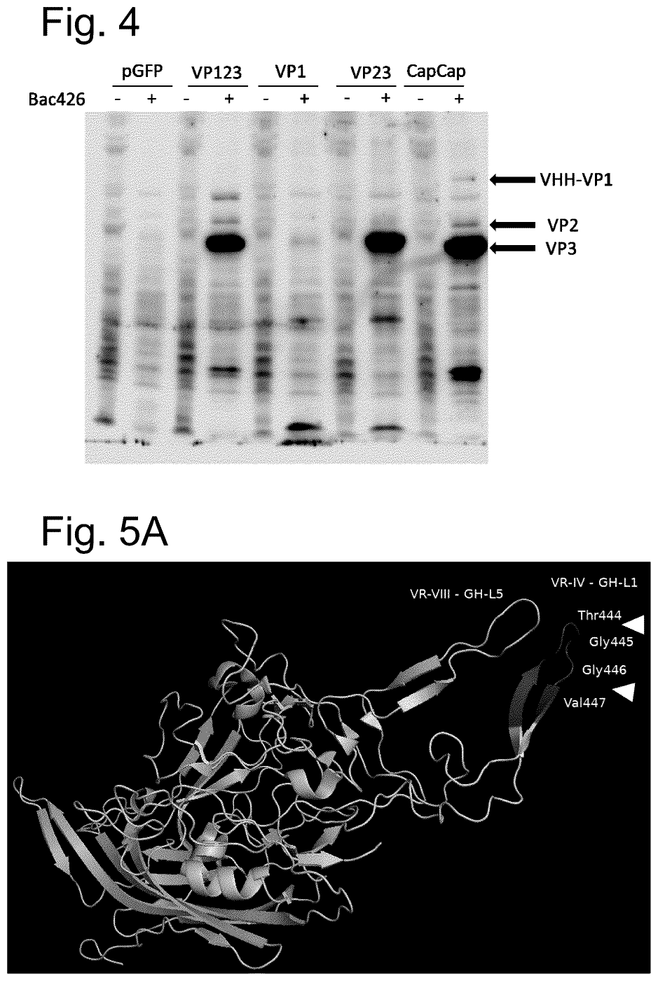

FIG. 5. (A) Cartoon representation of AAV5 VP1 3D structure in neutral conditions, resolved at 3.18 A (pdb 6JCT). The two outmost protrusions GH-L1 and GH-L5 are indicated. Chosen insertion sites are indicated with white arrows. (B) Alignment of VR-VI of AAV5 and AAV2 indicating that the AAV5 GH-L1 loop is shortened by 6 amino acids in comparison to AAV2 GH-L1.

FIG. 6. (A) Schematic overview of expression plasmids used for production of modified AAV in the HEK293T production platform. Red bar indicates insertion of either VHH or vNAR single domain antibody sequences. The VP1 translation initiation site of the VP2-VP3 expression plasmid is mutated in order to suppress expression of unmodified VP1. (B) SDS-PAGE analysis of AVB Sepharose purified AAV5 wild-type virions, AAV5 virions with VHH sdAb insertion (153 aa) and vNAR sdAb insertion (145 aa) between amino acids T444 and G445. (C) Genome copy quantification based on TaqMan quantitative PCR using SV40 PolyA-signal specific primers. (D) Comparison of SEAP activity resulting from Huh7 transduction using either AAV5 wild-type or sdAb-modified (vNAR and VHH) AAV5.

FIG. 7 (A) shows different CapCap design iterations used in transient transfection experiments of Sf+ insect cells. The top design (VP123) acts as a reference design encoding AAV5 wild-type viral proteins 1, 2 and 3. Middle (CapCap1; SEQ ID NO: 1) and bottom (CapCap2; SEQ ID NO: 77) design panels illustrate different promoter orientations used in order to in- or decrease expression levels of each respective ORF. CapCap1 utilizes the polH promoter to drive expression of VP1 containing a 161 amino-acid VHH modification and the p10 promoter to drive expression of wild-type VP2 and VP3. In the CapCap2 design this promoter orientation is reversed, making use of p10 to drive expression of modified VP1 and the polH promoter to drive expression of wild-type VP2 and VP3. Plasmids shown in (A) were used to transfect Sf+ cells that subsequently were infected with BacRep and BacTrans (SEAP transgene) baculovirus inoculant. Following incubation, rAAV was purified from cleared crude lysate using AVB Sepharose affinity resin. (B) shows SDS-PAGE of affinity purified rAAV with black arrows indicating different viral protein species. (C) shows viral titers (GC/ml) determined by qPCR in crude lysate and affinity purified virus batches. CapCap1 and CapCap2 both generate viral titers comparable to the VP123 reference design. (D) SEAP potency assay of VP123, CapCap1 and CapCap2 derived rAAV. Potency was assessed in two different HEK293T cell lines which either express or not express the receptor target for the CapCap-produced rAAV.

FIG. 8 shows production of CapCap1 and CapCap2 derived rAAV with CAG-eGFP as transgene. (A) shows the effect of promoter orientation on rAAV capsid stoichiometry based on SDS-PAGE analysis. (B) shows rAAV titers (GC/ml) in cleared crude lysate as well as affinity purified virus. (C) shows GFP-based potency assay. Target receptor positive and negative cells were co-cultured in different ratios and transduced with either AAV5 wild-type, CapCap1- or CapCap2-derived rAAV. Subsequently flow cytometry was used to determine transduction efficiencies in receptor positive or receptor negative cells.

### EXAMPLES

### Example 1

### 1.1. Materials & Methods

### 1.1.1 Plasmids

Plasmids used herein are listed as:

| Seq | |
|---|---|
| ID | |
| No. | Description |
| 1 | Transient transfection plasmid with AAV5 CapCap Design (Design |
| | A - short (20 bp) promoter interspacing) - p10 VP2-3 - polH VHH- |
| | VP1 |
| 2 | Transient transfection plasmid with single AAV5 Cap ORF - polH |
| | VP123 |
| 3 | Bac Recombination Plasmid for recombination with Bac.AMT05 |
| | containing AAV5 CapCap Design (Design A - short (20 bp) |
| | promoter interspacing) - p10 VP2-3 - polH VHH-VP1 |
| 4 | Bac Recombination Plasmid for recombination with Bac.AMT05 |
| | containing AAV5 CapCap Design (Design B - long (141 bp) |
| | promoter interspacing) - p10 VP2-3 - polH VHH-VP1 |
| 5 | HEK293T VP2-3 Expression plasmid |
| 6 | HEK293T VHH-VP1 Expression plasmid |
| 7 | HEK293T vNAR-VP1 Expression plasmid |

Plasmids were obtained from GeneArt (Thermo Fisher Scientific) that synthesized and subcloned the shown insert into the final CapCap plasmid. Plasmid identity was confirmed by Sanger sequencing and restriction digestion analysis.

1.1.2 rAAV Production in HEK293T Via Quadruple Transfection

HEK293T cells were seeded 24 hours prior to transfection in 150 mm2 petri dishes at final cell density of 10⁷ cells in a total of 25 ml complete DMEM (DMEM (Gibco)+10% FCS+1% PenStrep). 1 hour prior to transfection complete DMEM was replaced with 25 ml fresh complete DMEM. Quadruple transfection mixes were prepared by adding 4.5 pmol pHelper, 4.5 pmol VP2-3 Expression plasmid (SEQ ID NO: 5), 4.5 pmol VHH-VP1 or vNAR-VP1 (SEQ ID NO: 6 or 7, respectively) and 9 pmol pITR-SEAP to 0.9% NaCl solution. Equal volumes of DNA-NaCl solution and linear Polyethylenimine (25 kDa MW, Polysciences) solution (0.13 mg/ml) were incubated for 15 minutes at room-temperature and added to HEK293T culture medium. 72 hours post-transfection cells were lysed using 1× Lysis buffer (Lonza) and genomic DNA removed by Benzonase (Roche) digestion. Crude lysate was clarified by centrifugation at 1900×g for 15 minutes. AAV particles were bound in batch to AVB Sepharose HP resin (Cytiva LifeSciences) for 2 hours at room temperature and continuous shaking (85 rpm). AVB Sepharose HP resin was subsequently washed with PBS and bound particles eluted by addition of 0.2 M Glycine-HCL (pH2.5). Eluent was PH neutralized by addition of 0.5 M Tris/HCL (pH 8.5).

### 1.1.3 Transient Transfection and Transactivation of Express Sf+ Cells

Express Sf+ cells were cultured in Sf900 II medium (Thermo Fisher Scientific) at 28° C. Sf+ cells for small-scale expression were either cultured in 6-well plates without shaking or 125 ml shaker flasks with continuous rotary shaking at 135 rpm. Sf+ cells cultured in 6-well plates were seeded at 5×10⁵ cells/ml in a total volume of 1 ml. Sf+ cells cultured in 125 ml shaker flasks were seeded at 1.7×10⁶/ml in a total volume of 5 ml. Transfection mixes for 6-well plates were prepared by incubating 0.5 μg plasmid DNA with 1.5 μl Cellfectin II (Thermo Fisher Scientific) in a total volume of 120 μl 0.9% NaCl solution for 15 minutes at room-temperature. Transfection mixes for 125 ml shaker flasks were prepared by incubating 7.5 μg plasmid DNA with 22.5 μl Cellfectin II (Thermo Fisher Scientific) in a total volume of 1 ml 0.9% NaCl solution for 15 minutes at room temperature. Transfection mixes were slowly added to cell suspensions and homogenized by gently swirling. After 5 hours post-transfection, 9 ml of fresh, pre-warmed Sf900 II medium (Thermo Fisher Scientific) was added to the 125 ml shaker flasks. 6-well plates were incubated 16 hours at 28° C., 125 ml shaker flasks were incubated 72 hours at 28° C. and 135 rpm shaking prior to transactivation by addition of baculovirus. Transfected cells were transactivated after indicated incubation periods by addition of a BacTrans (SEQ ID NO: 68) at final concentrations of 1% (v/v) for 6-well plates or 1.5% (v/v) for 125 ml shaker flasks. Transfected and transactivated cells were harvested 48 hours post-infection for 6-well plate treatments or 72 hours post-infection for 125 ml shaker flask treatments.

For the production of genome containing rAAV 10 ml of Sf+ cell suspension (1.5e6/ml-1.5e7 cells total) was transfected with 15 ug of plasmid DNA. Transfection mixes were prepared by incubating 45 ul Cellfectin II (Thermo Fisher Scientific) and 15 ug of plasmid DNA in a total volume of 2 ml 0.9% NaCl solution for 15 minutes at room-temperature. Transfection mixes were slowly added to cell suspensions and homogenized by gently swirling. After 5 hours post-transfection, 18 ml of fresh, pre-warmed Sf900 II medium (Thermo Fisher Scientific) was added to the 125 ml shaker flasks and incubated for 72 hours at 28° C. and 135 rpm rotary shaking. Transfected cells were transactivated after 72 hours incubation by co-infection with Bac.ITR-SEAP (SEQ ID NO: 74) or Bac.ITR-eGFP (SEQ ID NO: 75) at final concentrations of 1% (v/v) and BacRep (SEQ ID NO: 76) at final concentration of 2% (v/v). Transfected and transactivated cells were harvested 72 hours post-infection and purified as described under batch-binding purification.

### 1.1.4 Production and Purification of AAV Via Baculovirus Expression System

AAV material was generated by volumetrically co-infecting expresSF+ insect cells with combinations of freshly amplified recombinant baculoviruses comprising resp. the two Cap expression cassettes (BacCapCap), BacRep (Bac.VD183 as described in WO2009/014445) and BacTrans (the SEAP transgene flanked by AAV2 ITRs, SEQ ID NO: 68). Following a 72 hour incubation at 28° C., cells were lysed in lysis buffer (1.5M NaCl, 0.5M Tris-HCl, 1 mM MgCl2, 1% Triton x-100, pH=8.5) for 1 hour. Next, genomic DNA was digested with benzonase (Merck) at 37 ºC for 1 hour after which cell debris was pelleted at 1900×g for 15 minutes (crude lysate samples). Supernatant was stored at 4° C. until the start of purification. AAV was then purified from crude lysed bulk (CLB) by batch binding with AVB sepharose (GE healthcare). In brief, AVB sepharose resin was washed in 0.2 M HPO4 pH=7.5 buffer, after which clarified crude lysate was added to the resin and incubated 2 hours at room temperature (RT) in an incubator shaking at 85 rpm. Resin was washed again in 0.2 M HPO4 pH=7.5 buffer. Next, bound virus was eluted from the resin with the addition of 0.2M Glycine pH=2.5. The pH of the eluted virus was immediately neutralized by the addition of 0.5M Tris-HCl PH=8.5 and stored at −20° C. until further use.

### 1.1.5 Western Blot Analysis

Samples from 6-well plates were harvest and lysed by aspiration of Sf900 II medium and addition of RIPA lysis buffer (Thermo Fisher Scientific) supplemented with cOmplete™ protease inhibitor cocktail (Roche). Contaminating DNA was removed by Benzonase (Roche) digestion. Samples were prepared for SDS PAGE analysis by addition of 1× Laemmli sample buffer. Proteins were denatured by boiling for 5 minutes at 95° C. Samples were run on 4-20% Mini-Protean TGX pre-cast (BioRad) gels for 45 minutes at 200 V constant voltage. Proteins were blotted by using the high-molecular weight species preset on the Trans-Blot Turbo Transfer system (BioRad). AAV5 VP1, VP2, VP3 were detected by addition of primary anti-VP123 antibodies (Progen) and secondary HRP-conjugated anti-mouse antibodies.

### 1.1.6 Batch-Binding Purification and Genome Copy Quantification of Recombinant AAV Particles and SDS PAGE Analysis

Samples from 125 ml shaker flasks were harvested and lysed by addition of lysis buffer (1× final concentration). Contaminating DNA was removed by Benzonase (Roche) digestion. Crude lysate was clarified by centrifugation at 1900×g for 15 minutes. AAV particles were bound in batch to AVB Sepharose HP resin (Cytiva LifeSciences) for 2 hours at room temperature and continuous shaking (85 rpm). AVB Sepharose HP resin was subsequently washed with PBS and bound particles eluted by addition of 0.2 M Glycine-HCL (pH2.5). Eluent was PH neutralized by addition of 0.5 M Tris/HCL (pH 8.5). Samples were prepared for SDS PAGE analysis by addition of 1× Laemmli sample buffer. Proteins were denatured by boiling for 5 minutes at 95° C. Samples were run on stain-free 4-20% Mini-Protean TGX pre-cast (BioRad) gels for 45 minutes at 200 V constant voltage. SDS-PAGE gels were developed in BioRad ChemiDoc MP Imaging System. The DNase-resistant AAV particle titers were determined using quantitative polymerase chain reaction (qPCR) with primers and probe directed against the promoter region.

### 1.1.7 In Vitro Functionality Assay Based on SEAP Expression

HEK293T cells (wild-type cells or cells stably expressing the receptor targeted by the modified VHH-VP1 protein) were seeded at 1e5 cells/well in 24-well plate. 24 hours after seeding, the culture medium was refreshed with Adenovirus 5 supplemented medium (MOI 50). rAAV as indicated was added to the cells either at 10⁴ GC/cell or 10⁵ GC/cell. 48 hours after infection, SEAP expression was measured in the supernatant using the SEAP Reporter Gene Assay (Roche) with an integration time of 1s.

1.1.8 In Vitro Functionality Assay Based on eGFP Expression

HEK293T cells (wild-type cells or cells stably expressing the receptor targeted by the VHH-modified VP1 protein) were mixed in different ratios to a final seeding density of 1e5 cells/well in 24-well plate. Receptor-expressing cells were added at either 10% or 90% final percentage. 24 hours after seeding, the culture medium was refreshed with Adenovirus 5 supplemented medium (MOI 30). rAAV was added at MOI 5e5 directly to wells after which cells were incubated for 48 hours. Cells were harvested for flow cytometric analysis by washing and resuspending cells in PBS buffer. In order to discriminate receptor positive and negative cells, the target-receptor encoding cells were stained using receptor-specific antibodies. Cell populations were subsequently quantified for the presence of eGFP (transgene) and VHH-target receptor.

### 1.2. Results

1.2.1 Production of AAV5 with Large Peptide Inserted Capsid from HEK293T Cells

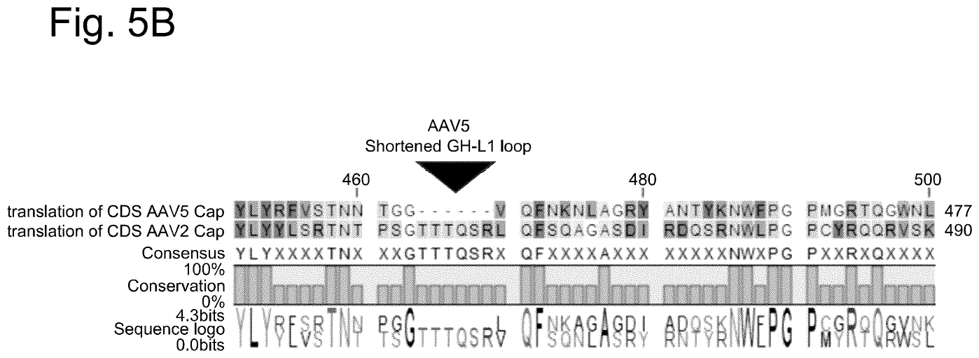

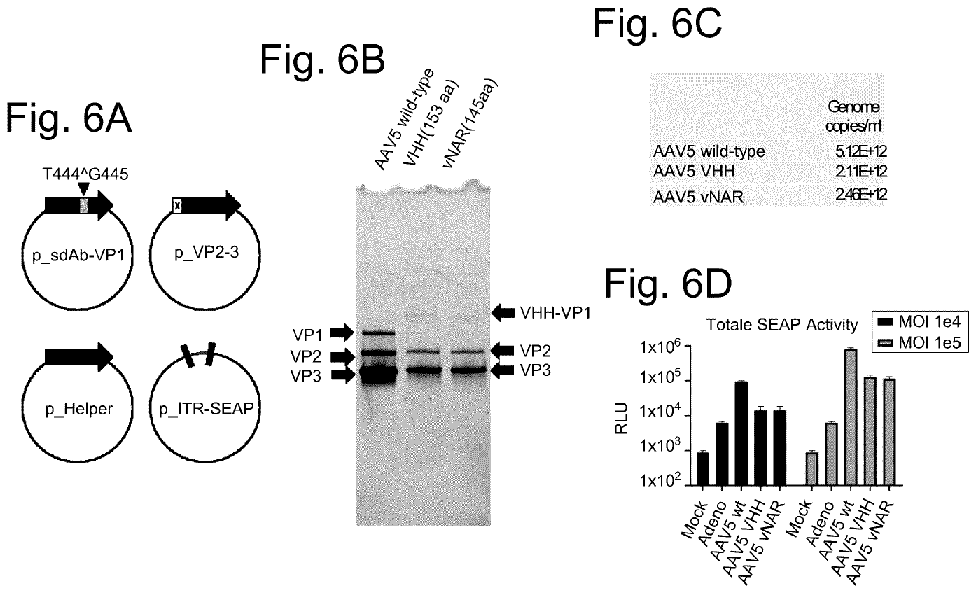

To see if large peptide insertion could be facilitated into AAV5 capsid, single domain antibody (sdAb) was used as the model. To test whether the first chosen insertion site (Thr444\hat{ }Gly445 in GH-L1 loop, FIG. 5(A)) would allow the co-expression of the modified VP1 and unmodified VP2-3, HEK293T cells were co-transfected with plasmids encoding AAV5-VP2-3 and a plasmid containing the modified AAV5-VP1. Additionally, a helper plasmid and a Rep plasmid were supplied (FIG. 6(A)). Virion assembly was assessed by AVB Sepharose affinity purification from clarified lysates. The chosen insertion site (Thr444\hat{ }Gly445 in GH-L1 loop, FIG. 5(A)) supported insertion of different sdAb sequences of ±150 amino acids as shown by expression in HEK293T cells using a quadruple transfection set up (FIG. 6(A)). Purification by AVB Sepharose affinity resin demonstrated assembly of viral particles bearing the sdAb-containing VP1 protein and wild-type VP2 and VP3 (FIG. 6(B)).

The SDS-PAGE analysis indicated a non-canonical stoichiometry of the capsid, deviating from the 1:1:10 (VP1:VP2:VP3) subunit distribution. This could be explained by a non-optimized quadruple transfection protocol, resulting in uneven distribution of plasmid DNA among recipient cells and hence insufficient expression of the modified VP1 protein. Optimization of plasmid DNA ratios and transfection reagents likely will lead to canonical capsid subunit stoichiometries. When quantifying the genome copy number using quantitative PCR, we found similar genome content in the modified AAV5 compared to the wild-type (FIG. 6(C)). In order to assess the potency of the modified AAV5 preparations, the activity of the transgene (secreted alkaline phosphatase) was measured in the supernatant of transduced Huh7 cells (FIG. 6(D)). Both modified AAV5 preparations were able to transduce Huh7, although less efficient than the wild-type reference. Considering the non-canonical stoichiometry of the modified AAV5 batches, we expect to restore the potency to similar levels as the wild-type when assembling the capsid with the canonical stoichiometry. However, due to the intrinsic challenge of transient transfection that would always favor more the combination of 2 or 3 plasmid per cell, robust production using quadruple plasmid would pose a challenge because of the increasing probability to produce VP23-only full particles.

### 1.2.2 CapCap Functionality in Insect Express Sf+ Cells Through Transient Transfection Method

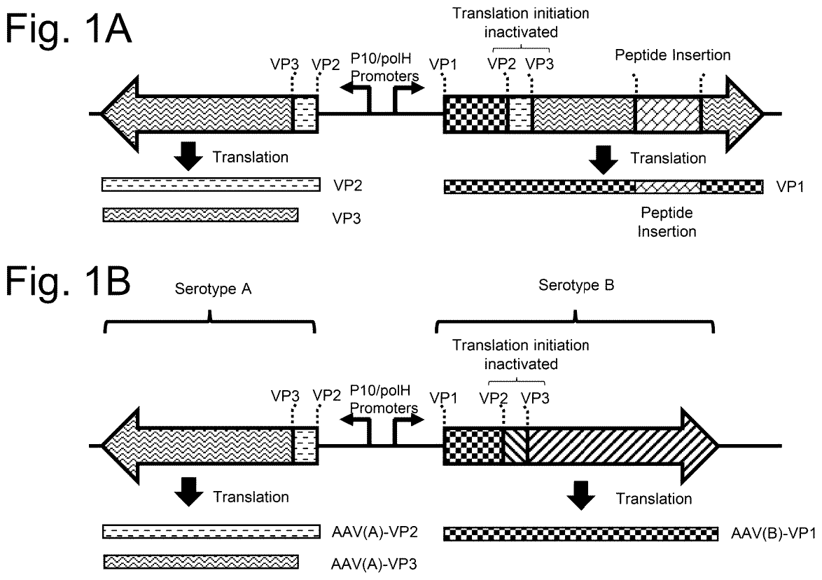

In order to assure all structural proteins of the large-peptide inserted AAV5 capsid would be produced in the same spatiotemporal, it is advantageous to express and regulate the gene expression by DNA sequences that act in cis. To facilitate this, the sdAb-VP1 (in this example the sdAb is a VHH) with the VP23 expression cassettes were combined to generate the CapCap concept (FIG. 1). To avoid the complexity of very large-sized plasmid generation or pHelper requirement in HEK based production, the CapCap concept can only be facilitated through the baculovirus expression system (BEVS) to produce the AAV.

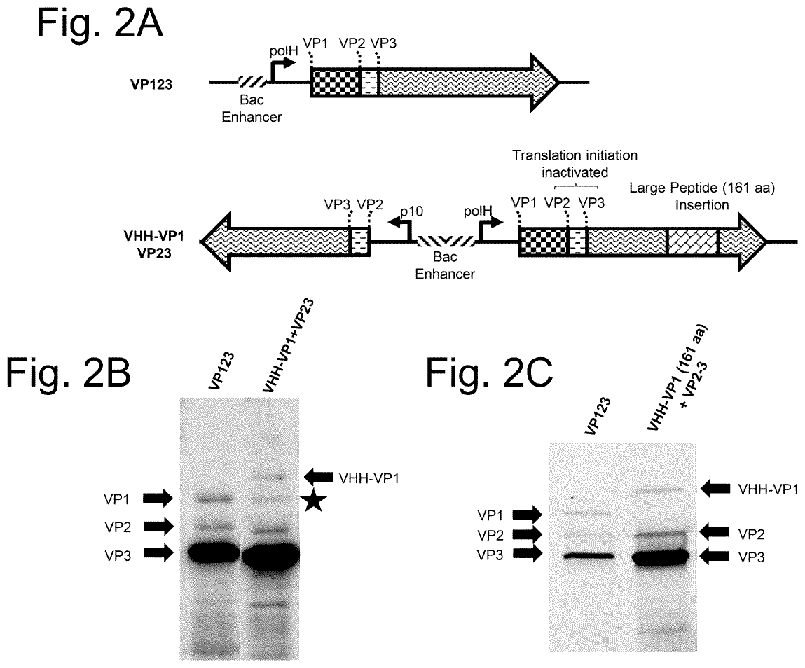

In order to obtain insights into the functionality of the CapCap construct as well as the insertion site Gly446\hat{ }Val447, transient transfection assays were employed to test expression of AAV5 VHH-VP1 as well as unmodified VP2 and VP3. In parallel to the CapCap construct, a reference construct containing the Cap open-reading frame (FIG. 2(A)), co-expressing the unmodified AAV5 VP1 with VP2 and VP3, was assessed. Our Western Blot analysis from small scale (6-well plate) transient transfection assays demonstrated that the CapCap construct sustained expression of the AAV5 VHH-VP1 in addition to unmodified AAV5 VP2 and VP3 (FIG. 2(B)). Comparing the Cap construct expression profile with the CapCap construct revealed a higher molecular weight species being expressed from the CapCap construct corresponding with the VHH-VP1 protein. In summary, the western blot analysis demonstrated expression of modified VP1 and unmodified VP2 and VP3 from the CapCap design.

Next, we were interested if the expression of the modified VHH-VP1 (Gly446\hat{ }Val447) with VP2 and VP3 would result in assembly of virions. Due to the absence of the Rep proteins and the ITR-flanked transgene, assembled particles would not contain viral genomes but assemble as empty particles. The employed batch-binding protocol relies on AVB Sepharose resin that specifically bound to assembled AAV virions. The purified AAV is therefore representative for the assembled virus population present in the crude lysate. SDS-PAGE analysis of purified AAV particles showed, as expected from previous Western Blot analysis, the presence of a higher molecular weight species of VP1 corresponding to the VHH-VP1 protein (FIG. 2(C)). In addition, we did not observe any Cathepsin protease cleavage products indicating that the sdAb insertion did not expose residues prone to Cathepsin cleavage (FIG. 2(C)). Estimating the molar ratios between VHH-VP1, VP2 and VP3 based on the band intensities showed a deviation from the consensus molar ratio (1:1:10 for VP1:VP2:VP3), indicating that expression levels of VHH-VP1 require optimization. Based on our preliminary data, we concluded that the CapCap design is capable of expressing modified VP1 and unmodified VP2 and VP3 that result in the assembly of empty virions. By modifying the translation initiation site of the modified VP1 we expected to increase expression levels and thereby shift the molar ratio of the capsid proteins towards the desired standard.

1.2.3 Generation of BacCapCap and Production of AAV5 with Large Peptide Inserted Capsid from Insect Sf+ Cells

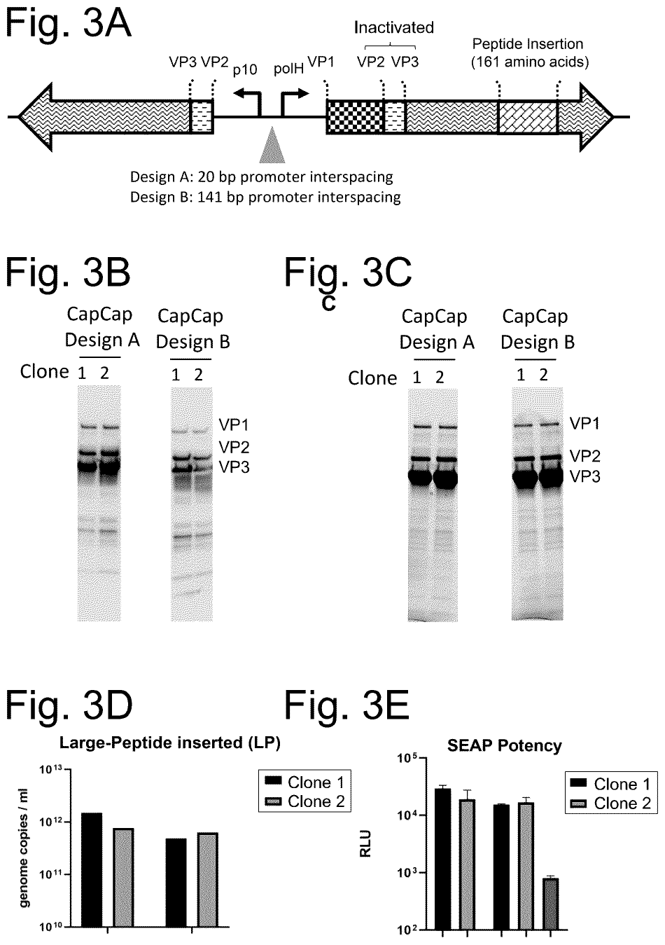

To see if the novel CapCap concept could be used to properly produce AAV particles, BacCapCap was generated and small transient recombinant AAV5 production experiments were performed in insect Express Sf+ cells. Intriguingly, the use of BacCapCap as designed in FIG. 3(A) could yield AAV particles with similar capsid profile like the transient transfection method with the same purification strategy (FIG. 3(B)). The purified DNase-resistant AAV5 particles also contained or packaged the transgene of interest as could be detected via qPCR (FIG. 3(D)). The same particles were also functional to transfer the gene of interest (SEAP) into Huh7, as representative target cells (FIG. 3(E)).

### 1.2.4 Other Design(s) for BacCapCap

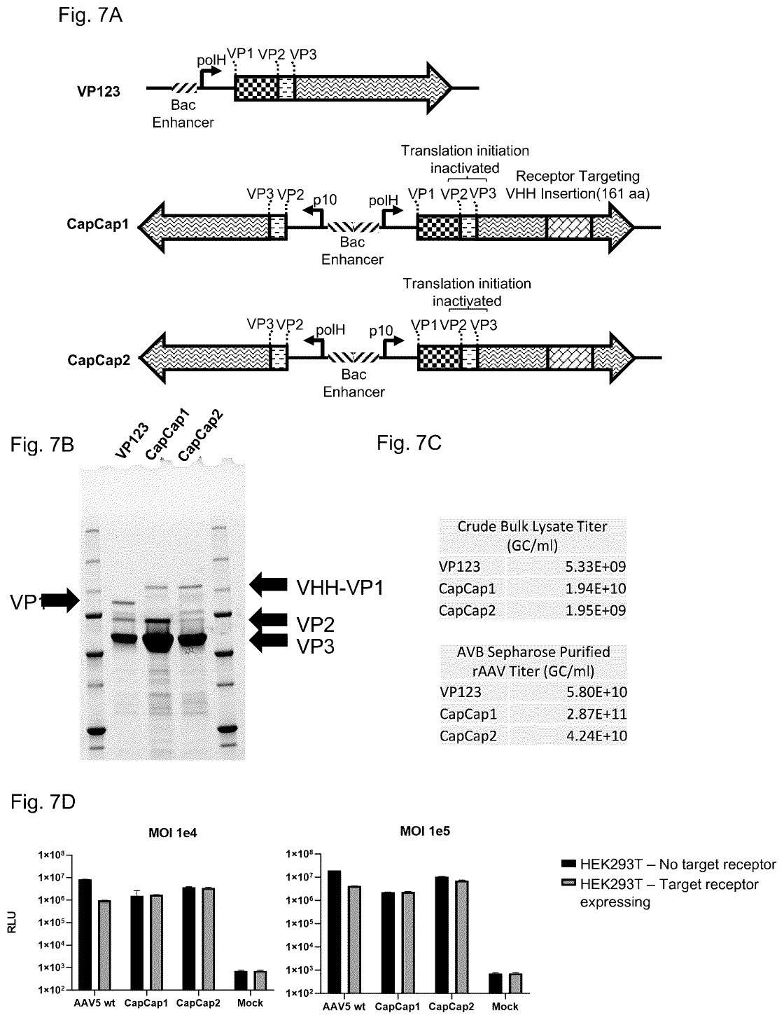

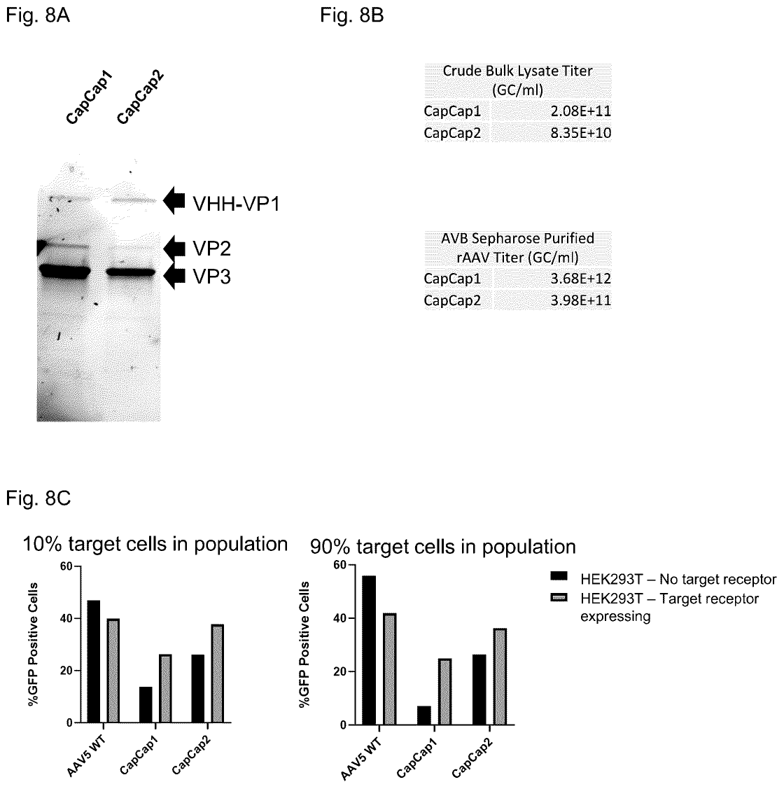

Different combination and/or orientation of promoters based on BacCapCap construct were tested for optimizing the VP1:2:3 stoichiometry based on the experimental protocols mentioned above. In the initial CapCap design (CapCap1; SEQ ID NO: 1) the p10 and polH promoters were used to drive expression of the two independent gene cassettes. It was established that using polH to drive expression of the modified VP1 protein resulted in non-canonical rAAV stoichiometries, with VP1 being underrepresented in the overall viral capsid population. In order to correct for this underrepresentation, it was chosen to exchange the promoter sequences between the independent ORFs. In this second design iteration (CapCap2; SEQ ID NO: 77) the stronger p10 promoter was used to drive the expression of modified VP1 while the relatively weaker polH promoter was used to drive the expression of VP2 and VP3 (FIG. 7(A)). The effect of exchanging the promoters between the two cassettes was compared side-by-side using the transient transfection method. In short, Sf+ cells were transfected with plasmids containing either CapCap1 or CapCap2 designs. After an incubation period to allow the cells to recover from transfection, the cell cultures were infected with two different baculoviruses (Bac-ITR and Bac-Rep) that provided the ITR-flanked SEAP-transgene and the replication proteins (Rep). rAAV particles were subsequently purified from cleared crude lysates using AVB Sepharose affinity purification. Upon analysis of purified rAAV by SDS PAGE it was found that using the weaker polH promoter to drive VP2-3 expression and the stronger p10 promoter to drive modified VP1 expression (CapCap2) resulted in capsid stoichiometries that resembles the VP123 stoichiometry of wild-type rAAV5 (FIG. 7 (B) and FIG. 8 (A)).

### 1.2.5 the Use of CapCap Design to Create AAV Capsid Displaying Two Different Serotypes

Expression and assembly of mosaic virions are tested by following the steps mentioned above in Transient Transfection and Transactivation of Express Sf+ cells.

Expression and assembly of mosaic virions, composed of, for example, AAV5-derived VP2 and VP3 as well as AAV9-derived VP1, are assessed in the transient transfection context. Molecular adjustments based on our findings of the transient transfection assays are translated into the generation of baculovirus seeds in order to prove functionality of the CapCap design in the baculovirus expression system.

### 1.2.6 the Use of CapCap Design to Create Genome-Packaging and Infectious Viral Particles

Viral titers of purified rAAV were determined by qPCR using transgene-promoter specific primer-probe combinations (FIG. 7(C) and FIG. 8(B)). Intriguingly, the CapCap2 design resulted in rAAV that tittered in a similar range as compared to the reference rAAV5 wild-type VP123 design. However, despite the non-canonical particle stoichiometry resulting from CapCap1 designs, rAAV derived from CapCap1 designs reached higher titers than both, AAV5 wild-type and CapCap2 derived material (FIG. 7(C)). Similarly, using a different Bac-ITR transgene (eGFP), rAAV derived from CapCap1 designs displayed higher titers than rAAV expressed from CapCap2 (FIG. 8 (B)). This observation can be explained by higher expression levels of VP2 and VP3 when using p10 as a promoter. VP2 and VP3 can assemble into viral particles and package genomes that are measured in the tittering qPCR, however, particles lacking VP1 are not infectious and hence qPCR-based titers are not entirely representative for infectivity of the purified virus.

In order to assess the infectivity of CapCap1 and CapCap2 derived rAAV, as well as testing functionality of the VP1 peptide insertion, two different HEK293T cell lines were infected at two different MOIs. Subsequently, the activity of the secreted alkaline phosphatase (SEAP) transgene was measured in the cell supernatant (FIG. 7 (D)). The VP1 modification consists of an epitope-binding VHH domain that specifically binds a receptor that is only present in one of the two HEK293T cell lines. Hence, infectivity was tested in the context of presence or absence of the rAAV target receptor. Interestingly, while rAAV5 wild-type was between 5 to 10-fold less efficient (depending on MOI used) in transducing target receptor positive cells, as measured by SEAP activity, both CapCap derived rAAV transduced receptor positive and negative cells with comparable efficiency. In addition, CapCap2 produced rAAV displayed approximately 5-fold higher transduction of both cell lines, indicating that capsids derived from CapCap2 are more infectious. Specificity of CapCap1 and CapCap2 produced rAAV was additionally tested using eGFP as transgene (FIG. 8 (C)). The aforementioned HEK293T receptor positive or receptor negative cells were mixed in different ratios, representing a situation in which target cells were either overrepresented (90% of total population) or underrepresented (10% of total population). Subsequently, mixed populations were transduced with either rAAV5 wild-type or CapCap1 or CapCap2 derived rAAV. Transduction was assessed by flow cytometry, detecting the target receptor as well as eGFP transgene expression. CapCap1 and CapCap2 derived rAAV preferably transduced cells expressing the target receptor. Interestingly, in both high and low abundance scenarios, the modified rAAV specifically targeted the receptor expressing cells, demonstrating functionality of the VHH-VP1 modification. Similarly to the SEAP-based assay, CapCap2 derived rAAV overall showed higher transduction efficiency compared to CapCap1, albeit CapCap1 rAAV being more target specific. In summary, both CapCap designs result in intact infectious viral particles that contain functional VHH peptide-modifications in their VP1. Due to lower production levels of VP2 and VP3, the CapCap2 design displays reduced productivity (lower GC/ml) compared to CapCap1. However, due to the higher abundance of VP1 in CapCap2 derived virions, this material shows higher potency—effectively compensating for the lower productivity.

1. An insect cell comprising one or more nucleic acid constructs comprising:
i) a first expression cassette comprising a first promoter operably linked to a nucleotide sequence encoding an mRNA, translation of which in the cell produces a parvoviral VP1 capsid protein; and,
ii) a second expression cassette comprising a second promoter operably linked to a nucleotide sequence encoding an mRNA, translation of which in the cell produces parvoviral VP2 and VP3 capsid proteins.

2. An insect cell according to claim 1, wherein at least one of:
a) the nucleotide sequence encoding the mRNA, translation of which in the cell produces only a parvoviral VP1 capsid protein, comprises at least one of:
i) a suboptimal translation initiation codon for the VP1 coding sequence;
ii) an inactivation of the native suboptimal translation initiation codon for the VP2 coding sequence; and,
iii) an inactivation of the native ATG translation initiation codon for the VP3 coding sequence,
and,
b) the nucleotide sequence encoding the mRNA, translation of which in the cell produces parvoviral VP2 and VP3 capsid proteins, comprises at least one of:
i) a deletion of the translation initiation codon for the VP1 coding sequence and optionally a deletion of at least a part of the VP1 coding sequence upstream of the VP2 initiation codon;
ii) a suboptimal translation initiation codon for the VP2 coding sequence; and,
iii) an ATG translation initiation codon for the VP3 coding sequence.

3. An insect cell according to claim 2, wherein in a) at least one of:
i) the suboptimal translation initiation codon for the VP1 coding sequence is an ACG, CTG, TTG or GTG codon or an ATG codon in combination with an upstream out-of-frame initiation codon;
ii) the native suboptimal translation initiation codon for the VP2 coding sequence is inactivated by replacement with another threonine codon; and,
iii) the native ATG translation initiation codon for the VP3 coding sequence is inactivation by its deletion or by replacement with a codon coding for conservative substitution of methionine, preferably leucine.

4. An insect cell according to claim 1, wherein the nucleotide sequence encoding the mRNA, translation of which in the cell produces only the parvoviral VP1 capsid protein, encodes a common amino acid sequence that has at least 90% amino acid sequence identity with a corresponding common amino acid sequence encoded in the nucleotide sequence encoding the mRNA, translation of which in the cell produces the parvoviral VP2 and VP3 capsid proteins, and wherein the parts in the nucleotide sequences that encode the common amino acid sequences have less than 90% nucleotide sequence identity.

5. An insect cell according to claim 1, wherein the first and second expression cassettes are:
a) both comprised in a single (episomal) nucleic acid construct, preferably a baculoviral vector; or,
b) both comprised in at least one nucleic acid construct that is integrated in the genome of the insect cell,
and wherein preferably, the first and second expression cassettes present in opposite directions of transcription.

6. An insect cell according to claim 1, wherein the first and second promoters are two different baculoviral promoters, preferably two different late or very late baculoviral promoters, more preferably two different baculoviral promoters selected from the group consisting of the polH, p10, p6.9 and pSel120 promoters, most preferably, the first promoter is the polH promoter and the second promoter is the p10 promoter.

7. An insect cell according to claim 1, wherein the parvoviral VP1 capsid protein is at least one of:
a) a parvoviral VP1 capsid protein of a different parvovirus or of a different serotype than the parvoviral VP2 and VP3 capsid proteins; and,
b) a parvoviral VP1 capsid protein comprising an insertion of an exogenous amino acid sequence.

8. An insect cell according to claim 7, wherein the parvoviral VP1 capsid protein comprises an insertion of an exogenous amino acid sequence in an exposed loop of the capsid protein, wherein preferably, the exposed loop is at least one of the GH-L1 loop and the GH-L5 loop.

9. An insect cell according to claim 7, wherein the exogenous amino acid sequence encodes a single domain antibody, a ligand, designed ankyrin repeat protein (DARPin), an anticalin, an HDL-binding epitope or a reporter protein.

10. An insect cell according to claim 9, wherein the single domain antibody, ligand, DARPin or anticalin has affinity for a cell surface marker that is specifically expressed on a target cell or target tissue, wherein preferably, the target cell or target tissue a central nervous system cell, a muscle cell, a liver cell, a synovial cell, a lymphocyte or a progenitor thereof, an endothelial cell, preferably a vascular endothelial cell, more preferably a vascular endothelial cell that is present in the blood brain barrier, or wherein the single domain antibody, ligand, DARPin or anticalin has affinity for HDL.

11. An insect cell according to claim 7, wherein:
a) the parvoviral VP1 capsid protein is an AAV5 capsid protein; or,
b) the parvoviral VP1 capsid protein is an AAV9 capsid protein and the parvoviral VP2 and VP3 capsid proteins are AAV5 capsid proteins.

12. An insect cell according to claim 1, wherein the insect cell further comprises at least one of:
iii) a nucleic acid construct comprising at least one expression cassette for expression of nucleotide sequence encoding parvoviral Rep proteins; and,
iv) a nucleic acid construct comprising a transgene that is flanked by at least one parvoviral inverted terminal repeat sequence,
wherein preferably at least one of the nucleic acid construct in iii) and the nucleotide sequence in iv) is comprised in a baculoviral vector.

13. A method for producing a recombinant parvoviral virion in a cell comprising the steps of:
a) culturing a cell as defined in claim 1 under conditions such that recombinant parvoviral virion is produced; and,
b) recovery of the recombinant parvoviral virion.

14. A method according to claim 13, wherein recovery of the recombinant parvoviral virion in step b) comprises at least one of affinity-purification of the virion using an immobilised anti-parvoviral antibody, preferably a single chain camelid antibody or a fragment thereof, or filtration over a filter having a nominal pore size of 30-70 nm.

15. A kit of parts comprising at least an insect cell as defined in claim 1 and the nucleic acid construct and/or at least one of:
iii) a nucleic acid construct comprising at least one expression cassette for expression of nucleotide sequence encoding parvoviral Rep proteins; and,
iv) a nucleic acid construct comprising a transgene that is flanked by at least one parvoviral inverted terminal repeat sequence.


SEQUENCE LISTING (77 sequences)

SEQ ID NO: 1 - DNA, 8771 bp, Artificial Sequence
  Transient transfection plasmid with AAV5 CapCap Design (Design A - short (20 bp) promoter interspacing) - p10 VP2-3 - polH VHH(P2X7)-VP1
gctaataacaggcctgctggtaatcgcaggcctttttatttggtgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgtagaaggaaggccgtcaaggcctaggcgcgccgtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgggtaccttaaaggggtcgggtaaggtatcgggttccgataggtctggtggttctgtattccccggtgctgtccggggcaaagtccacaaactgggggtcgttgtagttgtttgtgtactggatctctgggttccacctcttggagttttccttcttgagctcccactccatctccacggtgacctgcccggtgctgtactgggtgatgaagctgctgacgggcacgtccgagaagctggtgatatttccgggcacaggcgtgttcttgatgagcatcatgggcggtgggtgtttgagtccgaatccgcccatggccggagaggggtgaaagtgcgcccccgtctctgggatcttggcccagatgggtccttggaggtacacgtccctctccatccacacgctgccgggcacgatttcctggaggttgtacgtgccggtcgcgggggcagtggtggagctctggttgttggtggccatctgcccgccgacgttgtacgccacgcggttcaccggctgcgtctcgctctcgctggtgatgagcatgttgccctcgaggtacgtggcggtggtgcccgggttcgccggctggctgttgaagatcatagtgttctccagggcataggtgttgctgccctggaggttgttggtcatgccgttcggctgcgggggcacctggtaactcgcgccctcgagctccatcctattggtcgtggcgaaggcgctgacactggcgcggttgaccccggagcccaggttccagccctgggttcggcccatgggccccgggaaccagtttttgtaggtgttggcgtatctcccggccaggttcttgttgaactggactccgccagtgttatttgtgctcacgaagcggtacaagtactggtccaccagcgggttggccagcttgaacaggttctgactgggagcgaagctggagtggaagggcacctcctcaaagttgtaggtaaactcaaagttgttgcccgttctcagcatcttgctgggaaagtactctaggcagaagaagctgctcctctcggtgggattttctgtgttgtcgcggttcagcgtcgcgtaaccgtactgcggcagcgtaaagacctgcggagggaaggccggcaggcatccctcggtcccgttgccgacgacgtagggcagctggtagtcgtcgtccgtaaacacttggacggtggaggtgaggttgttggcgatggtggtggtggagtcctgcaccgtgacctctttgacttgaatgttgaagattttgactctgagggaccggggtctgaagccccagtagttgttgatgagtctttgccagtctcgggggctccagtggctgtggaagcggttaaagtcaaagtacccccagggggtgctgtatccaaagtaggcgttggcgttgcttccgtcgacggagccgcttttgatctctcggtactggtggttgttgtagctgggcagcacccaggttcgggtggacttggtgacgactctgtcccccatccacgtggaatcgcaatgccaatctcccgaggcattgcccactccatcggcaccttggttattgtcgcccaatgggccgccacctcccgcagacattgtatcagctcccaaacttgaggctggttgggctgggatttgcagctgctgggatccgctgggtccagcttcggcgtctgacgaggtggaaggcttggagtcctcttcggtccgagccttctttctttttggaaagtggtcgtctatccgctttccggtaggggccgtcttaacaggaccggtatccgcgaagggcgaattccagcacactggcggccgttactagtgagcgggccgctttcgaatctaggggtgatcaagtcttcgtcgagtgattgtaaataaaatgtaatttacagtatagtattttaattaatatacaaatgatttgataataattcttatttaactataatatattgtgttgggttgaattaaaggtccgtatcatgaggccggccgttttacgcgtagaattctacccgtaaagcgagtttagttatgagccatgtgcaaaacatgacatcagcttttatttttataacaaatgacatcatttcttgattgtgttttacacgtagaattctactcgtaaagcgagttcagttttgaaaaacaaatgacatcatctttttgattgtgctttacaagtagaattctacccgtaaatcaagttcggttttgaaaaacaaatgagtcatattgtatgatatcatattgcaaaacaaatgactcatcaatcgatcgtgcgttacacgtagaattctactcgtaaagcgagtttatgagccgtgtgcaaaacatgacatcatctcgatttgaaaaacaaatgacatcatccactgatcgtgcattacaagtagaattctactcgtaaagccagttcggttatgagccgtgtacaaaacatgacatcagattatgactcatacttgattgtgttttacgcgtagaattctactcgtaaagcggaccgcatgcaggaccgctttacgagtagaattttacttgtaaaacacaatcaagaaatgatgtcatttttgtacgtgattataaacatgtttaaacatggtacattgaacttaatttttgcaagttgataaacatgattaatgtacgactcatttgtttgtgcaagttgataaacgtgattaatatatgactcatatgtttgtgcaaaaatgatgtcatcgtacaaactcgctttacgagtagaattctacttgtaacgcatgatcaagggatgatgtcatttgtttttttaaaattcaactcgctttacgagtagaattctacttgtaaaacacaatcgagggatgatgtcatttgtagaatgatgtcatttgtttttcaaaaccgaactcgctttacgagtagaattctacttgtaacgcaagatcggtggatgatgtcattttaaaaatgatgtcatcgtacaaactcgctttacgagtagaattctacgtgtaaaactctagatgtaatgagacgcacaaactaatatcacaaactggaaatgtctatcaatatatagttgctgatctatgcatcagctgctagtactccggaatattaatagatcatggagataattaaaatgataaccatctcgcaaataaataagtattttactgttttcgtaacagttttgtaataaaaaaacctataaatattccggattattcataccgtcccaccatcgggcgcggatcgtaccgggcccaagcttcctgttaagacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacagctcccacaggcaaaagaatcgatgatcatttccccaagcggaaaaaagccagaacagaggaagatagcaaacccagcacaagctccgatgctgaggcaggtccatctggttcacaacaactccagattcctgctcagcctgctagctctctcggcgccgacaccttaagcgctggcggcggaggacccctcggagataacaatcagggcgctgacggcgtcggaaacgctagcggcgactggcactgtgacagcacatggatgggcgatagggtggtgacaaaaagcacaaggacatgggtcctcccttcttataataatcatcaatatagagaaattaagtccggaagcgtggatggctctaatgctaatgcttatttcggctattccacaccttggggatatttcgatttcaatagatttcattcccattggtcccctagggattggcagaggctgattaataattactggggatttaggcctagaagcctgagggtgaagatttttaatatccaggtgaaggaagtgaccgtccaagatagcacaacaacaattgctaataatctgacatcaacagtgcaggtcttcacagatgatgattatcaactcccttatgtggtgggaaatggcacagaaggctgtctccccgcttttccacctcaagtgttcacactccctcaatatggatatgccacactcaatagagataataccgagaaccctacagaaagatcctcctttttttgtttggaatatttcccttccaaaatgctcaggacaggaaataatttcgaattcacatataatttcgaagaagtcccttttcatagctcctttgccccttcccaaaatctctttaaactcgctaatcctctcgtcgatcaatatctctataggtttgtctccaccaacaataccggaggcgccggtggtggtggatcgggaggaggaggttcgggaggtggaggttctggaggcggtggcagcggaggcggcggttcagaggtacaattggtagaatcaggaggaaaattggtgcaggcaggaggctctctgaggctgtcgtgcagcgccagcggtcgcaccttttcgtttagtacctctacgatgggatggttcaggcaggcccctggtaaagaactcgagtttgtagccgcaattgactggtcggatttcaacacctattacgctgatagcgtaaagggaagatttacaatctcaaggcataacccgaggaattcagtgtatctccaactcaacagccttaaaccggaggacacggccgtctattactgcgctgctcatagtgaaacgcgcggtggaactcgctactttgataggccatcgttgtataactactggggccagggtacgcaagtgactgtctcatcgggaggcggaggcgccgttcaattcaataagaatctcgctggcaggtatgctaatacatataagaattggtttcctggccccatgggaaggacacaaggatggaatctcggaagcggagtgaatagagcttctgtgtccgcttttgctaccacaaacagaatggaactggaaggagccagctatcaagtccctcctcaacccaatggaatgacaaataatctgcaaggatccaatacatacgctctcgaaaataccatgatttttaattcccaacccgctaatcccggaacaacagctacatatctggaaggaaatatgctgattacatccgaatccgaaacccaacctgtcaatagagtcgcctataatgtgggaggacaaatggctacaaataatcaatccagcacaacagctcctgctacaggaacctataatctgcaagagattgtccctggttccgtctggatggaaagagatgtctatctgcagggccctatttgggctaaaattcccgaaacaggagcccatttccatcctagccctgctatgggaggctttggcctgaagcatcctccacctatgatgctgattaaaaataccccagtccctggcaacattacatcctttagcgatgtccctgtgtcctcctttattacacaatattccacaggccaagtgacagtcgaaatggaatgggaactgaaaaaagagaatagcaaaagatggaatcccgaaattcaatataccaataattataatgatcctcaattcgtcgatttcgctcctgattccacaggagagtataggacaacaaggcccattggcacaagatatctgacaagacctctgtgaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggccgcttggatcccagaattctaggtacctcttaattaactggcctcatgggccttccttctatcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaaataaaacgaaaggcccagtcttccgactgagcctttcgttttattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaa

SEQ ID NO: 2 - DNA, 7572 bp, Artificial Sequence
  Transient transfection plasmid with single AAV5 Cap ORF - polH VP123
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgagggcccgcggtaccgtcgatgaatccatggttcgcaattctgcacaggtgaagaccaagcaacacggacttaggcaccaggttttctggtcatgcaccatgtacggggaccctctggcacttctacatccgccgttactgtgaatccgaggcgttcgtagaagggcagattgcgaggagcacttgtttccaagaatgccggtacgcctgccctctccgccgcctctacaccaggcagcacgaccgcagagcccaagccctttccttggtgatcagggctgacacctactgtagccagaaaccacgcaggctctttcgggcggtgaggagcgagcaagccctccatttgctgttgtgcggcgagcctcgagcccgaaagttcagccatacgcgggcctatctcggcgaataccgcaccggcttcgacactttccggcgtcgtccaaacggccactgctgctccgtcgtcagctacccacaccttacctatatccaaaccgacacgagtgaggaacaactcctgcagttcagtcacacgctctatgtggcgatcaggatctacggtatgccttgtagctgggtaatcagcgaaagctgcagcaagtgtacgcacggcacgggggacatcatcgcgtgtcgccaatctaacagtcggtttgtactcagtcatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatttggccgcgaattcactagtgattgcggaataattgccatatgtaaatgatgtcatcgttctaactcgctttacgagtagaattctacgtgtaaaacataatcaagagatgatgtcatttgtttttcaaaactgaactcaagaaatgatgtcatttgtttttcaaaactgaactggctttacgagcagaattctacttgtaacgcatgatcaagggatgatgtcatttgttttttaaaattgaactggctttacgagtagaattctacttgtaaaacacaatcgagagatgatgtcatattttgcacacggctctaattaaactcgctttacgagtaaaattctacttgtaacgcatgatcaagggatgatgtcattggatgagtcatttgtttttcaaaactaaactcgctttacgagtagaattctacttgtaaaacacaatcaagggatgatgtcattatacaaatgatgtcatttgtttttcaaaactaaactcgctttacgggtagaattctacttgtaaaacacaatcgagggatgatgtcatcctttacacatgattataaacgtgtttatgtatgactcatttgtttttcaaaactaaactcgctttacgagtagaattctacttgtaacgcacgatcaagggatgatgtcatttatttgtgcaaagctgatgtcatcttttgcacacgattataaacacaatcaaataatgactcatttgttttcaaaactgaactcgctttacgagtagaattctacttgtaaaacacaatcaagggatgatgtcattttaaaaatgatgtcatttgtttttcaaaactaaactcgctttacgagtagaattctacgtgtaaaacacaatcaagggatgatgtcatttactaaaataaaataattatttaaataaaaatgtttttattgtaaaatacacattgattacacgtgacaatcgaattcccgcttgctagcttcttaagttagatctttatgcatttcggagcgagaccatcatggagataattaaaatgataaccatctcgcaaataaataagtattttactgttttcgtaacagttttgtaataaaaaaacctataaatattccggattattcataccgtcccaccatcgggcgcggatcgtaccgggcccaagcttcctgttaagacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaaaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 3 - DNA, 14171 bp, Artificial Sequence
  Bac Recombination Plasmid for recombination with Bac.AMT05 containing AAV5 CapCap Design (Design A - short (20 bp) promoter interspacing) - p10 VP2-3 - polH VHH(P2X7)-VP1
catggagatctgagctcggcgcgccgtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgggtaccttaaaggggtcgggtaaggtatcgggttccgataggtctggtggttctgtattccccggtgctgtccggggcaaagtccacaaactgggggtcgttgtagttgtttgtgtactggatctctgggttccacctcttggagttttccttcttgagctcccactccatctccacggtgacctgcccggtgctgtactgggtgatgaagctgctgacgggcacgtccgagaagctggtgatatttccgggcacaggcgtgttcttgatgagcatcatgggcggtgggtgtttgagtccgaatccgcccatggccggagaggggtgaaagtgcgcccccgtctctgggatcttggcccagatgggtccttggaggtacacgtccctctccatccacacgctgccgggcacgatttcctggaggttgtacgtgccggtcgcgggggcagtggtggagctctggttgttggtggccatctgcccgccgacgttgtacgccacgcggttcaccggctgcgtctcgctctcgctggtgatgagcatgttgccctcgaggtacgtggcggtggtgcccgggttcgccggctggctgttgaagatcatagtgttctccagggcataggtgttgctgccctggaggttgttggtcatgccgttcggctgcgggggcacctggtaactcgcgccctcgagctccatcctattggtcgtggcgaaggcgctgacactggcgcggttgaccccggagcccaggttccagccctgggttcggcccatgggccccgggaaccagtttttgtaggtgttggcgtatctcccggccaggttcttgttgaactggactccgccagtgttatttgtgctcacgaagcggtacaagtactggtccaccagcgggttggccagcttgaacaggttctgactgggagcgaagctggagtggaagggcacctcctcaaagttgtaggtaaactcaaagttgttgcccgttctcagcatcttgctgggaaagtactctaggcagaagaagctgctcctctcggtgggattttctgtgttgtcgcggttcagcgtcgcgtaaccgtactgcggcagcgtaaagacctgcggagggaaggccggcaggcatccctcggtcccgttgccgacgacgtagggcagctggtagtcgtcgtccgtaaacacttggacggtggaggtgaggttgttggcgatggtggtggtggagtcctgcaccgtgacctctttgacttgaatgttgaagattttgactctgagggaccggggtctgaagccccagtagttgttgatgagtctttgccagtctcgggggctccagtggctgtggaagcggttaaagtcaaagtacccccagggggtgctgtatccaaagtaggcgttggcgttgcttccgtcgacggagccgcttttgatctctcggtactggtggttgttgtagctgggcagcacccaggttcgggtggacttggtgacgactctgtcccccatccacgtggaatcgcaatgccaatctcccgaggcattgcccactccatcggcaccttggttattgtcgcccaatgggccgccacctcccgcagacattgtatcagctcccaaacttgaggctggttgggctgggatttgcagctgctgggatccgctgggtccagcttcggcgtctgacgaggtggaaggcttggagtcctcttcggtccgagccttctttctttttggaaagtggtcgtctatccgctttccggtaggggccgtcttaacaggaccggtatccgcgaagggcgaattccagcacactggcggccgttactagtgagcgggccgctttcgaatctaggggtgatcaagtcttcgtcgagtgattgtaaataaaatgtaatttacagtatagtattttaattaatatacaaatgatttgataataattcttatttaactataatatattgtgttgggttgaattaaaggtccgtaagtactacgcgtgctctagatgtaatgagacgcacaaactaatatcacaaactggaaatgtctatcaatatatagttgctgatctatgcatcagctgctagtactccggaatattaatagatcatggagataattaaaatgataaccatctcgcaaataaataagtattttactgttttcgtaacagttttgtaataaaaaaacctataaatattccggattattcataccgtcccaccatcgggcgcggatcgtaccgggcccaagcttcctgttaagacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacagctcccacaggcaaaagaatcgatgatcatttccccaagcggaaaaaagccagaacagaggaagatagcaaacccagcacaagctccgatgctgaggcaggtccatctggttcacaacaactccagattcctgctcagcctgctagctctctcggcgccgacaccttaagcgctggcggcggaggacccctcggagataacaatcagggcgctgacggcgtcggaaacgctagcggcgactggcactgtgacagcacatggatgggcgatagggtggtgacaaaaagcacaaggacatgggtcctcccttcttataataatcatcaatatagagaaattaagtccggaagcgtggatggctctaatgctaatgcttatttcggctattccacaccttggggatatttcgatttcaatagatttcattcccattggtcccctagggattggcagaggctgattaataattactggggatttaggcctagaagcctgagggtgaagatttttaatatccaggtgaaggaagtgaccgtccaagatagcacaacaacaattgctaataatctgacatcaacagtgcaggtcttcacagatgatgattatcaactcccttatgtggtgggaaatggcacagaaggctgtctccccgcttttccacctcaagtgttcacactccctcaatatggatatgccacactcaatagagataataccgagaaccctacagaaagatcctcctttttttgtttggaatatttcccttccaaaatgctcaggacaggaaataatttcgaattcacatataatttcgaagaagtcccttttcatagctcctttgccccttcccaaaatctctttaaactcgctaatcctctcgtcgatcaatatctctataggtttgtctccaccaacaataccggaggcgccggtggtggtggatcgggaggaggaggttcgggaggtggaggttctggaggcggtggcagcggaggcggcggttcagaggtacaattggtagaatcaggaggaaaattggtgcaggcaggaggctctctgaggctgtcgtgcagcgccagcggtcgcaccttttcgtttagtacctctacgatgggatggttcaggcaggcccctggtaaagaactcgagtttgtagccgcaattgactggtcggatttcaacacctattacgctgatagcgtaaagggaagatttacaatctcaaggcataacccgaggaattcagtgtatctccaactcaacagccttaaaccggaggacacggccgtctattactgcgctgctcatagtgaaacgcgcggtggaactcgctactttgataggccatcgttgtataactactggggccagggtacgcaagtgactgtctcatcgggaggcggaggcgccgttcaattcaataagaatctcgctggcaggtatgctaatacatataagaattggtttcctggccccatgggaaggacacaaggatggaatctcggaagcggagtgaatagagcttctgtgtccgcttttgctaccacaaacagaatggaactggaaggagccagctatcaagtccctcctcaacccaatggaatgacaaataatctgcaaggatccaatacatacgctctcgaaaataccatgatttttaattcccaacccgctaatcccggaacaacagctacatatctggaaggaaatatgctgattacatccgaatccgaaacccaacctgtcaatagagtcgcctataatgtgggaggacaaatggctacaaataatcaatccagcacaacagctcctgctacaggaacctataatctgcaagagattgtccctggttccgtctggatggaaagagatgtctatctgcagggccctatttgggctaaaattcccgaaacaggagcccatttccatcctagccctgctatgggaggctttggcctgaagcatcctccacctatgatgctgattaaaaataccccagtccctggcaacattacatcctttagcgatgtccctgtgtcctcctttattacacaatattccacaggccaagtgacagtcgaaatggaatgggaactgaaaaaagagaatagcaaaagatggaatcccgaaattcaatataccaataattataatgatcctcaattcgtcgatttcgctcctgattccacaggagagtataggacaacaaggcccattggcacaagatatctgacaagacctctgtgaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggccgccccgggggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccat

SEQ ID NO: 4 - DNA, 14292 bp, Artificial Sequence
  Bac Recombination Plasmid for recombination with Bac.AMT05 containing AAV5 CapCap Design (Design B - long (141 bp) promoter interspacing) - p10 VP2-3 - polH VHH(P2X7)-VP1
catggagatctgagctcggcgcgccgtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgggtaccttaaaggggtcgggtaaggtatcgggttccgataggtctggtggttctgtattccccggtgctgtccggggcaaagtccacaaactgggggtcgttgtagttgtttgtgtactggatctctgggttccacctcttggagttttccttcttgagctcccactccatctccacggtgacctgcccggtgctgtactgggtgatgaagctgctgacgggcacgtccgagaagctggtgatatttccgggcacaggcgtgttcttgatgagcatcatgggcggtgggtgtttgagtccgaatccgcccatggccggagaggggtgaaagtgcgcccccgtctctgggatcttggcccagatgggtccttggaggtacacgtccctctccatccacacgctgccgggcacgatttcctggaggttgtacgtgccggtcgcgggggcagtggtggagctctggttgttggtggccatctgcccgccgacgttgtacgccacgcggttcaccggctgcgtctcgctctcgctggtgatgagcatgttgccctcgaggtacgtggcggtggtgcccgggttcgccggctggctgttgaagatcatagtgttctccagggcataggtgttgctgccctggaggttgttggtcatgccgttcggctgcgggggcacctggtaactcgcgccctcgagctccatcctattggtcgtggcgaaggcgctgacactggcgcggttgaccccggagcccaggttccagccctgggttcggcccatgggccccgggaaccagtttttgtaggtgttggcgtatctcccggccaggttcttgttgaactggactccgccagtgttatttgtgctcacgaagcggtacaagtactggtccaccagcgggttggccagcttgaacaggttctgactgggagcgaagctggagtggaagggcacctcctcaaagttgtaggtaaactcaaagttgttgcccgttctcagcatcttgctgggaaagtactctaggcagaagaagctgctcctctcggtgggattttctgtgttgtcgcggttcagcgtcgcgtaaccgtactgcggcagcgtaaagacctgcggagggaaggccggcaggcatccctcggtcccgttgccgacgacgtagggcagctggtagtcgtcgtccgtaaacacttggacggtggaggtgaggttgttggcgatggtggtggtggagtcctgcaccgtgacctctttgacttgaatgttgaagattttgactctgagggaccggggtctgaagccccagtagttgttgatgagtctttgccagtctcgggggctccagtggctgtggaagcggttaaagtcaaagtacccccagggggtgctgtatccaaagtaggcgttggcgttgcttccgtcgacggagccgcttttgatctctcggtactggtggttgttgtagctgggcagcacccaggttcgggtggacttggtgacgactctgtcccccatccacgtggaatcgcaatgccaatctcccgaggcattgcccactccatcggcaccttggttattgtcgcccaatgggccgccacctcccgcagacattgtatcagctcccaaacttgaggctggttgggctgggatttgcagctgctgggatccgctgggtccagcttcggcgtctgacgaggtggaaggcttggagtcctcttcggtccgagccttctttctttttggaaagtggtcgtctatccgctttccggtaggggccgtcttaacaggaccggtatccgcgaagggcgaattccagcacactggcggccgttactagtgagcgggccgctttcgaatctaggggtgatcaagtcttcgtcgagtgattgtaaataaaatgtaatttacagtatagtattttaattaatatacaaatgatttgataataattcttatttaactataatatattgtgttgggttgaattaaaggtccgtaatgcccgggcagctcacggagattaccgaccaatctatctgatcggcgacactttgtgtggtactggagcggagaggtaactatgatgccgctaacaacctctctgtcgtcgctaacgtttatagtctagggccggcctcttgtaatgagacgcacaaactaatatcacaaactggaaatgtctatcaatatatagttgctgatctatgcatcagctgctagtactccggaatattaatagatcatggagataattaaaatgataaccatctcgcaaataaataagtattttactgttttcgtaacagttttgtaataaaaaaacctataaatattccggattattcataccgtcccaccatcgggcgcggatcgtaccgggcccaagcttcctgttaagacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacagctcccacaggcaaaagaatcgatgatcatttccccaagcggaaaaaagccagaacagaggaagatagcaaacccagcacaagctccgatgctgaggcaggtccatctggttcacaacaactccagattcctgctcagcctgctagctctctcggcgccgacaccttaagcgctggcggcggaggacccctcggagataacaatcagggcgctgacggcgtcggaaacgctagcggcgactggcactgtgacagcacatggatgggcgatagggtggtgacaaaaagcacaaggacatgggtcctcccttcttataataatcatcaatatagagaaattaagtccggaagcgtggatggctctaatgctaatgcttatttcggctattccacaccttggggatatttcgatttcaatagatttcattcccattggtcccctagggattggcagaggctgattaataattactggggatttaggcctagaagcctgagggtgaagatttttaatatccaggtgaaggaagtgaccgtccaagatagcacaacaacaattgctaataatctgacatcaacagtgcaggtcttcacagatgatgattatcaactcccttatgtggtgggaaatggcacagaaggctgtctccccgcttttccacctcaagtgttcacactccctcaatatggatatgccacactcaatagagataataccgagaaccctacagaaagatcctcctttttttgtttggaatatttcccttccaaaatgctcaggacaggaaataatttcgaattcacatataatttcgaagaagtcccttttcatagctcctttgccccttcccaaaatctctttaaactcgctaatcctctcgtcgatcaatatctctataggtttgtctccaccaacaataccggaggcgccggtggtggtggatcgggaggaggaggttcgggaggtggaggttctggaggcggtggcagcggaggcggcggttcagaggtacaattggtagaatcaggaggaaaattggtgcaggcaggaggctctctgaggctgtcgtgcagcgccagcggtcgcaccttttcgtttagtacctctacgatgggatggttcaggcaggcccctggtaaagaactcgagtttgtagccgcaattgactggtcggatttcaacacctattacgctgatagcgtaaagggaagatttacaatctcaaggcataacccgaggaattcagtgtatctccaactcaacagccttaaaccggaggacacggccgtctattactgcgctgctcatagtgaaacgcgcggtggaactcgctactttgataggccatcgttgtataactactggggccagggtacgcaagtgactgtctcatcgggaggcggaggcgccgttcaattcaataagaatctcgctggcaggtatgctaatacatataagaattggtttcctggccccatgggaaggacacaaggatggaatctcggaagcggagtgaatagagcttctgtgtccgcttttgctaccacaaacagaatggaactggaaggagccagctatcaagtccctcctcaacccaatggaatgacaaataatctgcaaggatccaatacatacgctctcgaaaataccatgatttttaattcccaacccgctaatcccggaacaacagctacatatctggaaggaaatatgctgattacatccgaatccgaaacccaacctgtcaatagagtcgcctataatgtgggaggacaaatggctacaaataatcaatccagcacaacagctcctgctacaggaacctataatctgcaagagattgtccctggttccgtctggatggaaagagatgtctatctgcagggccctatttgggctaaaattcccgaaacaggagcccatttccatcctagccctgctatgggaggctttggcctgaagcatcctccacctatgatgctgattaaaaataccccagtccctggcaacattacatcctttagcgatgtccctgtgtcctcctttattacacaatattccacaggccaagtgacagtcgaaatggaatgggaactgaaaaaagagaatagcaaaagatggaatcccgaaattcaatataccaataattataatgatcctcaattcgtcgatttcgctcctgattccacaggagagtataggacaacaaggcccattggcacaagatatctgacaagacctctgtgaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggccgccccgggggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccat

SEQ ID NO: 5 - DNA, 7294 bp, Artificial Sequence
  CR5_AgeI_AL256 AAV5 Cap VP1 START ATG->TAG
atcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggttagtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccggttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgccgat

SEQ ID NO: 6 - DNA, 7753 bp, Artificial Sequence
  CR5_AgeI_AL256 - AAV5 SpliceMT(G-T)+ linker TMEM30 linker
atcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccatattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagactgcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacattatctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactgccggcggaggcggatctggcggaggcggaagtggcggcggaggaagcggagggggcggatctggggggggaggctcagaagtgcagcttcaagcatctggaggtggcctggttcaagcaggaggcagtcttcgcttgtcctgcgccgctagtggatttaaaattacccactacacgatgggttggttccgacaggccccaggcaaggagagagaattcgtctctcgcattacctggggaggagacaacactttttactcaaattccgttaaaggtcgtttcaccatctcacgcgataacgccaagaacacggtatacctgcagatgaactccttaaaaccagaggataccgcagattattactgtgctgcaggctcgaccagcacagccactccacttagagttgactactggggcaaggggactcaggtgactgtcagtagtggaggtggaggcgccggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccggttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgccgat

SEQ ID NO: 7 - DNA, 7729 bp, Artificial Sequence
  CR5_AgeI_AL256 - AAV5 SpliceMT(G-T)+ linker CD71 VNAR 8.5 linker
atcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccatattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagactgcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacattatctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactgccggcggaggcggatctggcggaggcggaagtggcggcggaggaagcggagggggcggatctggggggggaggctcagcgagagtggaccagactcctcagactataactaaggagactggagagagtttaaccattaactgcgtgttacgcgacagcaactgcgcactgccttcaacgtattggtatcgaaagaagtctggatccacaaacgaagagtccatttctaagggcggacggtatgtggagacagttaatagcgggtctaaatcgttttctctgaggattaacgacttgacagtggaagattcaggaacatacaggtgtaaggtcattgcccagcttggctggtggttacgaggatgtaactaccgaaaacatgacgtgtacggggatggcacagccgtgaccgtcaacgcaggaggtggaggcgccggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccggttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgccgat

SEQ ID NO: 8 - protein, 736 aa, adeno-associated virus serotype 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 9 - protein, 599 aa, adeno-associated virus serotype 1
TAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 10 - protein, 534 aa, adeno-associated virus serotype 1
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 11 - protein, 735 aa, adeno-associated virus serotype 2 [sequence omitted]

SEQ ID NO: 12 - protein, 598 aa, adeno-associated virus serotype 2
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 13 - protein, 533 aa, adeno-associated virus serotype 2
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - protein, 736 aa, adeno-associated virus serotype 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 15 - protein, 599 aa, adeno-associated virus serotype 3
TAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 16 - protein, 534 aa, adeno-associated virus serotype 3
MASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 17 - protein, 734 aa, adeno-associated virus serotype 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 18 - protein, 598 aa, adeno-associated virus serotype 4
TAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 19 - protein, 538 aa, adeno-associated virus serotype 4
MRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 20 - protein, 724 aa, adeno-associated virus serotype 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 21 - protein, 588 aa, adeno-associated virus serotype 5
TAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 22 - protein, 532 aa, adeno-associated virus serotype 5
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 23 - protein, 736 aa, adeno-associated virus serotype 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 24 - protein, 599 aa, adeno-associated virus serotype 6
TAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 25 - protein, 534 aa, adeno-associated virus serotype 6
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 26 - protein, 737 aa, adeno-associated virus serotype 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 27 - protein, 600 aa, adeno-associated virus serotype 7
TAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 28 - protein, 534 aa, adeno-associated virus serotype 7
VAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 29 - protein, 738 aa, adeno-associated virus serotype 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 30 - protein, 601 aa, adeno-associated virus serotype 8
TAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 31 - protein, 535 aa, adeno-associated virus serotype 8
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 32 - protein, 736 aa, adeno-associated virus serotype 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 599 aa, adeno-associated virus serotype 9
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - protein, 534 aa, adeno-associated virus serotype 9
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - protein, 738 aa, adeno-associated virus serotype 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 36 - protein, 601 aa, adeno-associated virus serotype 10
TAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 37 - protein, 535 aa, adeno-associated virus serotype 10
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 38 - protein, 733 aa, adeno-associated virus serotype 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 39 - protein, 596 aa, adeno-associated virus serotype 11
TAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 40 - protein, 537 aa, adeno-associated virus serotype 11
MRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 41 - protein, 742 aa, adeno-associated virus serotype 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 42 - protein, 605 aa, adeno-associated virus serotype 12
TAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 43 - protein, 537 aa, adeno-associated virus serotype 12
MRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 44 - DNA, 2178 bp, Artificial Sequence
  Cap AAV2/5
  CDS (1)..(2178)
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 45 - protein, 725 aa, Artificial Sequence
  Synthetic Construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 46 - DNA, 2208 bp, Artificial Sequence
  AAV 1; VP1, VP2, VP3; startcodon VP1 altered (GTG)
gtggctgccgacggttatctacccgattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctg

SEQ ID NO: 47 - protein, 18 aa, Artificial Sequence
  Flexible linker
KESGSVSSEQLAQFRSLD

SEQ ID NO: 48 - protein, 14 aa, Artificial Sequence
  Flexible linker
EGKSSGSGSESKST

SEQ ID NO: 49 - protein, 8 aa, Artificial Sequence
  PON1_P1_ApoA1-epitope
GKQLNLKL

SEQ ID NO: 50 - protein, 7 aa, Artificial Sequence
  PON1 P2 ApoA1 epitope
TLSEKAK

SEQ ID NO: 51 - protein, 11 aa, Artificial Sequence
  LCAT s108 ApoA1 epitope
TYSVEYLDSSK

SEQ ID NO: 52 - protein, 6 aa, Artificial Sequence
  LCAT K249 ApoA1 epitope
LKEEQR

SEQ ID NO: 53 - protein, 11 aa, Artificial Sequence
  ABCA1 ApoA1 epitope 1
RYVWGGFAYLQ

SEQ ID NO: 54 - protein, 9 aa, Artificial Sequence
  epitope 1 r587
FEDMRYVWG

SEQ ID NO: 55 - protein, 9 aa, Artificial Sequence
  epitpe 1 q597
FAYLQDVVE

SEQ ID NO: 56 - protein, 30 aa, Artificial Sequence
  ABCA1 epitope 2
CPPGAGGLPPPQRKQNTADILQDLTGRNIS

SEQ ID NO: 57 - protein, 10 aa, Artificial Sequence
  C1477 surround epitope
MLPVCPPGAG

SEQ ID NO: 58 - protein, 9 aa, Artificial Sequence
  S1506 surround epitope
GRNISDYLV

SEQ ID NO: 59 - protein, 9 aa, Artificial Sequence
  apoB LDL-receptor binding epitope
RLTRKRGLK

SEQ ID NO: 60 - DNA, 1194 bp, Artificial Sequence
  Rep52 wt
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagaccgctaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 61 - DNA, 1194 bp, Artificial Sequence
  rep52 sf9 (insect cell) optimised
atggagctggtgggttggctggtggacaagggtatcacctccgagaagcagtggatccaggaggaccaggcttcctacatctccttcaacgctgcttccaactcccgttcccagatcaaggctgctctggacaacgctggtaagatcatgtccctgaccaagaccgctcctgactacctggtgggtcagcagcctgtggaggacatctcctccaaccgtatctacaagatcctggagctgaacggttacgaccctcagtacgctgcttccgtgttcctgggttgggctaccaagaagttcggtaagcgtaacaccatctggctgttcggtcctgctaccaccggtaagaccaacatcgctgaggctatcgctcacaccgtgcctttctacggttgcgtgaactggaccaacgagaacttccctttcaacgactgcgtggacaagatggtgatctggtgggaggagggtaagatgaccgctaaggtggtggagtccgctaaggctatcctgggtggttccaaggtgcgtgtggaccagaagtgcaagtcctccgctcagatcgaccctacccctgtgatcgtgacctccaacaccaacatgtgcgctgtgatcgacggtaactccaccaccttcgagcaccagcagcctctgcaggaccgtatgttcaagttcgagctgacccgtcgtctggaccacgacttcggtaaggtgaccaagcaggaggtgaaggacttcttccgttgggctaaggaccacgtggtggaggtggagcacgagttctacgtgaagaagggtggtgctaagaagcgtcctgctccttccgacgctgacatctccgagcctaagcgtgtgcgtgagtccgtggctcagccttccacctccgacgctgaggcttccatcaactacgctgaccgttaccagaacaagtgctcccgtcacgtgggtatgaacctgatgctgttcccttgccgtcagtgcgagcgtatgaaccagaactccaacatctgcttcacccacggtcagaaggactgcctggagtgcttccctgtgtccgagtcccagcctgtgtccgtggtgaagaaggcttaccagaagctgtgctacatccaccacatcatgggtaaggtgcctgacgcttgcaccgcttgcgacctggtgaacgtggacctggacgactgcatcttcgagcagtaa

SEQ ID NO: 62 - DNA, 1194 bp, Artificial Sequence
  rep52 AT optimised
atggaattagtaggatggttagtagataaaggaataacatcagaaaaacaatggatacaagaagatcaagcatcatatatatcatttaatgcagcatcaaattcaagatcacaaataaaagcagcattagataatgcaggaaaaataatgtcattaacaaaaacagcaccagattatttagtaggacaacaaccagtagaagatatatcatcaaatagaatatataaaatattagaattaaatggatatgatccacaatatgcagcatcagtatttttaggatgggcaacaaaaaaatttggaaaaagaaatacaatatggttatttggaccagcaacaacaggaaaaacaaatatagcagaagcaatagcacatacagtaccattttatggatgtgtaaattggacaaatgaaaattttccatttaatgattgtgtagataaaatggtaatatggtgggaagaaggaaaaatgacagcaaaagtagtagaatcagcaaaagcaatattaggaggatcaaaagtaagagtagatcaaaaatgtaaatcatcagcacaaatagatccaacaccagtaatagtaacatcaaatacaaatatgtgtgcagtaatagatggaaattcaacaacatttgaacatcaacaaccattacaagatagaatgtttaaatttgaattaacaagaagattagatcatgattttggaaaagtaacaaaacaagaagtaaaagatttttttagatgggcaaaagatcatgtagtagaagtagaacatgaattttatgtaaaaaaaggaggagcaaaaaaaagaccagcaccatcagatgcagatatatcagaaccaaaaagagtaagagaatcagtagcacaaccatcaacatcagatgcagaagcatcaataaattatgcagatagatatcaaaataaatgttcaagacatgtaggaatgaatttaatgttatttccatgtagacaatgtgaaagaatgaatcaaaattcaaatatatgttttacacatggacaaaaagattgtttagaatgttttccagtatcagaatcacaaccagtatcagtagtaaaaaaagcatatcaaaaattatgttatatacatcatataatgggaaaagtaccagatgcatgtacagcatgtgatttagtaaatgtagatttagatgattgtatatttgaacaataa

SEQ ID NO: 63 - DNA, 1194 bp, Artificial Sequence
  rep52 GC optimised
atggagctggtggggtggctggtggacaaggggatcacgagcgagaagcagtggatccaggaggaccaggcgagctacatcagcttcaacgcggcgagcaacagccggagccagatcaaggcggcgctggacaacgcggggaagatcatgagcctgacgaagacggcgccggactacctggtggggcagcagccggtggaggacatcagcagcaaccggatctacaagatcctggagctgaacgggtacgacccgcagtacgcggcgagcgtgttcctggggtgggcgacgaagaagttcgggaagcggaacacgatctggctgttcgggccggcgacgacggggaagacgaacatcgcggaggcgatcgcgcacacggtgccgttctacgggtgcgtgaactggacgaacgagaacttcccgttcaacgactgcgtggacaagatggtgatctggtgggaggaggggaagatgacggcgaaggtggtggagagcgcgaaggcgatcctgggggggagcaaggtgcgggtggaccagaagtgcaagagcagcgcgcagatcgacccgacgccggtgatcgtgacgagcaacacgaacatgtgcgcggtgatcgacgggaacagcacgacgttcgagcaccagcagccgctgcaggaccggatgttcaagttcgagctgacgcggcggctggaccacgacttcgggaaggtgacgaagcaggaggtgaaggacttcttccggtgggcgaaggaccacgtggtggaggtggagcacgagttctacgtgaagaaggggggggcgaagaagcggccggcgccgagcgacgcggacatcagcgagccgaagcgggtgcgggagagcgtggcgcagccgagcacgagcgacgcggaggcgagcatcaactacgcggaccggtaccagaacaagtgcagccggcacgtggggatgaacctgatgctgttcccgtgccggcagtgcgagcggatgaaccagaacagcaacatctgcttcacgcacgggcagaaggactgcctggagtgcttcccggtgagcgagagccagccggtgagcgtggtgaagaaggcgtaccagaagctgtgctacatccaccacatcatggggaaggtgccggacgcgtgcacggcgtgcgacctggtgaacgtggacctggacgactgcatcttcgagcagtaa

SEQ ID NO: 64 - DNA, 1194 bp, Artificial Sequence
  Extreme codon optimized Rep52
atggaattggtgggttggcttgtcgataaaggcataacgagcgaaaaacaatggattcaagaagatcaagcatcctatattagctttaacgctgcgagtaatagcagaagccagataaaagcagcgttggacaacgctggtaagatcatgtcgcttacaaagacggcgccggactatctcgtcggtcaacaaccagttgaggacataagttcaaacagaatttacaagatcttggagctcaatggttatgaccctcagtacgctgcgtctgtattcctcggatgggctacaaagaaatttggtaaacgcaatactatttggctcttcggtccagccacaaccggcaaaactaatattgccgaagctattgctcataccgtcccattttatggttgtgtgaattggactaacgaaaatttcccttttaatgattgcgtggataaaatggtcatatggtgggaagaaggtaaaatgacagctaaagtggttgaatccgcgaaggcaatcctgggtggttccaaagtccgtgtcgatcaaaagtgtaaaagctccgctcaaatcgatcctaccccggtaattgttacgagtaatactaatatgtgcgcagttattgacggtaactcgacgacgtttgaacatcaacaacccctccaggatagaatgtttaagtttgagctgactagacgcctcgaccacgatttcggtaaagttactaaacaggaggtgaaggatttctttcgctgggctaaagaccatgtcgtagaagtcgagcacgagttttatgttaagaaaggaggtgctaaaaagcgtcctgctcctagcgatgctgacatctccgaacctaagcgtgtccgtgaaagcgtcgcacaacctagcaccagcgatgctgaggcctccattaattatgctgatcgttatcagaataagtgcagccgccatgtcggtatgaacctcatgctcttcccttgtcgtcagtgtgaacgtatgaaccaaaactcaaacatatgttttacccatggtcaaaaggactgcctggagtgcttccctgtaagtgaatcgcaacctgtaagtgtggtaaagaaagcgtaccaaaagctctgttatatccaccacattatgggcaaagtaccggatgcctgtaccgcgtgtgaccttgtgaacgttgacctggacgattgtatattcgagcagtaa

SEQ ID NO: 65 - DNA, 1194 bp, Artificial Sequence; identical to SEQ ID NO: 60
  Rep52

SEQ ID NO: 66 - DNA, 1873 bp, Artificial Sequence
  Rep78
cgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaa

SEQ ID NO: 67 - DNA, 28 bp, artificial
  hr 28-mer
ctttacgagtagaattctacgcgtaaaa

SEQ ID NO: 68 - DNA, 12538 bp, Artificial Sequence
  Bac Trans
cgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccaggctgcagggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgataatacgactcactatagggagacccaagcttgatcccccctcttcctcctcctcaagggaaagctgcccacttctagctgccctgccatcccctttaaagggcgacttgctcagcgccaaaccgcggctccagccctctccagcctccggctcagccggctcatcagtcggtcaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttgtccgtgttgcttggtcttcacctgtgcagaattgcgaaccatggattcatcgacggtaccgcgggccctcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccccctgcagcctgcattaatgaatcggccaacgcgcggggagaggcggtttgcgtattgggcgctcttccgcttcctcgctcactgactcgctgcgctcggtcgttcggctgcggcgaggggggtaccagatcccatgggagctctgcagaattctctagaggcctcgcgagatcttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggccgacacccatttgaaaaaagcgaagacgaaatgttggacttgaatagcatgaagcgtcgtcagcaatacaatgacattggcgttttaaaacacgttcgtaacgttaacgctcgtgactttgtgtactgcctaacaagatacaacatagattgtagactcacaaattacaaacaaattataaaacatgagtttttgtcgtaaaaatgccacttgttttacgagtagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgat

SEQ ID NO: 69 - DNA, 1767 bp, Artificial Sequence
  AAV5 coding sequence for VP2 and VP3
acggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 70 - DNA, 2178 bp, Artificial Sequence
  AAV5 VP1 coding sequence with codon shuffled common sequence
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacagctcccacaggcaaaagaatcgatgatcatttccccaagcggaaaaaagccagaacagaggaagatagcaaacccagcacaagctccgatgctgaggcaggtccatctggttcacaacaactccagattcctgctcagcctgctagctctctcggcgccgacaccttaagcgctggcggcggaggacccctcggagataacaatcagggcgctgacggcgtcggaaacgctagcggcgactggcactgtgacagcacatggatgggcgatagggtggtgacaaaaagcacaaggacatgggtcctcccttcttataataatcatcaatatagagaaattaagtccggaagcgtggatggctctaatgctaatgcttatttcggctattccacaccttggggatatttcgatttcaatagatttcattcccattggtcccctagggattggcagaggctgattaataattactggggatttaggcctagaagcctgagggtgaagatttttaatatccaggtgaaggaagtgaccgtccaagatagcacaacaacaattgctaataatctgacatcaacagtgcaggtcttcacagatgatgattatcaactcccttatgtggtgggaaatggcacagaaggctgtctccccgcttttccacctcaagtgttcacactccctcaatatggatatgccacactcaatagagataataccgagaaccctacagaaagatcctcctttttttgtttggaatatttcccttccaaaatgctcaggacaggaaataatttcgaattcacatataatttcgaagaagtcccttttcatagctcctttgccccttcccaaaatctctttaaactcgctaatcctctcgtcgatcaatatctctataggtttgtctccaccaacaataccggaggcgttcaattcaataagaatctcgctggcaggtatgctaatacatataagaattggtttcctggccccatgggaaggacacaaggatggaatctcggaagcggagtgaatagagcttctgtgtccgcttttgctaccacaaacagaatggaactggaaggagccagctatcaagtccctcctcaacccaatggaatgacaaataatctgcaaggatccaatacatacgctctcgaaaataccatgatttttaattcccaacccgctaatcccggaacaacagctacatatctggaaggaaatatgctgattacatccgaatccgaaacccaacctgtcaatagagtcgcctataatgtgggaggacaaatggctacaaataatcaatccagcacaacagctcctgctacaggaacctataatctgcaagagattgtccctggttccgtctggatggaaagagatgtctatctgcagggccctatttgggctaaaattcccgaaacaggagcccatttccatcctagccctgctatgggaggctttggcctgaagcatcctccacctatgatgctgattaaaaataccccagtccctggcaacattacatcctttagcgatgtccctgtgtcctcctttattacacaatattccacaggccaagtgacagtcgaaatggaatgggaactgaaaaaagagaatagcaaaagatggaatcccgaaattcaatataccaataattataatgatcctcaattcgtcgatttcgctcctgattccacaggagagtataggacaacaaggcccattggcacaagatatctgacaagacctctgtga

SEQ ID NO: 71 - protein, 44 aa, Artificial Sequence
  translation of CDS AAV5 Cap
YLYRFVSTNNTGGVQFNKNLAGRYANTYKNWFPGPMGRTQGWNL

SEQ ID NO: 72 - protein, 50 aa, Artificial Sequence
  translation of CDS AAV2 Cap
YLYYLSRTNTPSGTTTQSRLQFSQAGASDIRDQSRNWLPGPCYRQQRVSK

SEQ ID NO: 73 - protein, 50 aa, Artificial Sequence
  Consensus
  misc_feature (4)..(7): Xaa can be any naturally occurring amino acid
  misc_feature (10)..(12): Xaa can be any naturally occurring amino acid
  misc_feature (20)..(20): Xaa can be any naturally occurring amino acid
  misc_feature (23)..(26): Xaa can be any naturally occurring amino acid
  misc_feature (28)..(35): Xaa can be any naturally occurring amino acid
  misc_feature (38)..(38): Xaa can be any naturally occurring amino acid
  misc_feature (42)..(43): Xaa can be any naturally occurring amino acid
  misc_feature (45)..(45): Xaa can be any naturally occurring amino acid
  misc_feature (47)..(50): Xaa can be any naturally occurring amino acid
YLYXXXXTNXXXGTTTQSRXQFXXXXAXXXXXXXXNWXPGPXXRXQXXXX

SEQ ID NO: 74 - DNA, 12538 bp, Artificial Sequence; identical to SEQ ID NO: 68
  Bac.ITR-SEAP

SEQ ID NO: 75 - DNA, 11073 bp, Artificial Sequence
  Bac.ITR-eGFP
cgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccaggctgcagggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgataatacgactcactatagggagacccaagcttgatcccccctcttcctcctcctcaagggaaagctgcccacttctagctgccctgccatcccctttaaagggcgacttgctcagcgcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacctcgagggaagaacctggttgctatggcaatagaaggggtggtatacttccttctgaccctgctcatccaacaccattttttcctcacccggtggatcgctgagcctgctagagagcccgtttttgatgaagatgacgatgtggctgaagaaagacaaagagttatgagtgggggaaataaaaccgacatcttaaagctaaacgaactaaccaaggtttactcaggctcttccagtccagcagtagatcggttatgtgttggagttcgacctggagagtgctttggcctcctgggagtgaatggtgcaggcaaaaccaccacattcaagatgctcactggggacaccacagtgacatcgggggatgctactgtggcaggcaagagcattttaactagtatttctgatgtccatcaaaacatgggctactgtcctcagtttgatgcaatcgatgacctgctcacaggcagagaacacctttacctctatgccaggctgcggggcgtgccatcgaaggaaatcgagaaggttgccaactggggtatccagagcctgggcttgtctctctacgctgaccgcctggcaggcacctacagtggaggcaataagaggaaactctctacagccatagctctcactggctgccctcccctgctgctgctggatgagcccacgacagggatggatccccaggcacgccgcatgctgtggaacaccattgtgagcatcatcagagaagggagagctgtggttctcacctcccacagcatggaagaatgtgaagctctgtgtacgcggctggccatcatggtgaagggcacctttcagtgtctgggtaccatccaacacctcaagtacaagtttggagacggctacattgtcacaatgaaaatcaaatctccaaaggacgacttgcttcccgatttgaatcctgtggagcagttcttccagggcaacttccctggcagcgtgcagagggagagacaccacagcatgctccagttccaggtcccctcgtcctccctggccaggatcttccagctgctcatttcccacaaggacagcctgctcatcgaggagtactcagtcacccagaccacgctggaccaggtgtttgtaaacttcgctaaacagcagactgagacctatgacctccctctgcacccccgggctgctggagccagctggcaagccaagcttgaagagaaatccggacggctgcagacacaggagcctttgcccgcaggatctgaacaactggctaacggaagcaaccccacggcagcagaagataagcacaccaggagtccacagtgagctggaggagactctttcagaaagaaaccaaaattcactggctcaccggaactcctgatggcaaaactgacctatcagaatggtcacctgtttatccagaaccctcgagtcgagttaagggcgaattcagatctgatatcgggggggggggatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccccctgcagcctgcattaatgaatcggccaacgcgcggggagaggcggtttgcgtattgggcgctcttccgcttcctcgctcactgactcgctgcgctcggtcgttcggctgcggcgaggggggtaccagatcccatgggagctctgcataattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggccgacacccatttgaaaaaagcgaagacgaaatgttggacttgaatagcatgaagcgtcgtcagcaatacaatgacattggcgttttaaaacacgttcgtaacgttaacgctcgtgactttgtgtactgcctaacaagatacaacatagattgtagactcacaaattacaaacaaattataaaacatgagtttttgtcgtaaaaatgccacttgttttacgagtagtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgat

SEQ ID NO: 76 - DNA, 13365 bp, Artificial Sequence
  BacRep
gtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagtcgagattaccctgttatccctaccagtgtgttggatttattgttcaaagatacagtcatccaaatccacattaaccagatcgcaggcagtacaagcgtctggcacttttcccatgatatgatgaatatagcataatttttgatacgccttttttacgacagaaacgggttgagattctgacaccggaaagcattctaaacagtctttctggccgtgagtgaaacagatattactattctgattcattctctcacattgtctgcagggaaacaacattaagttcatgcctacgtgacgagaacatttgttttggtagcggtctgcgtagtttatcgaagcttccgcatctgacgtgcttggctgcgcaaccgattctctcactcgtttgggctcacttatatctgcatcactcggggcgggtcttttcttagcaccaccttttttgacgtaaaattcatgttccacttcaacaacgtgatcctttgcccaacgaaagaagtctttgacttcttgttttgttaccttgccaaaatcatgatccagtcggcgcgtcaattcaaatttgaacattcggtcttgcaacggttgttggtgttcgaatgtcgtactgttaccgtcaatcacggcgcacatgttcgtgttgcttgtaacgatcaccggtgtcgggtctatctgcgcagagcttttgcatttctggtctacgcgcactttgctgcctcctaaaattgctttggccgactccacgactttagcggtcattttgccttcctcccaccaaataaccatcttgtcgacacagtcgttgaatggaaagttctcattggtccagttaacgcagccataaaaaggtacagtgtgggctatggcctccgctatgtttgtttttcccgtagttgcaggtccaaacaaccaaatggtgtttcttttgccaaactttttcgtcgcccagcccaaaaatacggaagccgcatattgaggatcgtagccgtttaactccaaaatcttatagatgcgattgctggaaatgtcttccacgggttgctggcccaccaggtagtcgggggcggttttagtcaggctcataatcttgcccgcattgtccaaggcagctttgatttggctacgcgagttggatgccgcattaaacgagatgtatgaggcttgatcttcttgtatccattgcttctccgaggtaatacccttgtccaccaaccaaccgaccaattccatggcgaccgagatccgcgcccgatggtgggacggtatgaataatccggaatatttataggtttttttattacaaaactgttacgaaaacagtaaaatacttatttatttgcgagatggttatcattttaattatctccatgatctattaatattccggagtactgctagcaccatggatcccggtccgaagcgcgcggaattcaaaggcctacgtcgacgagctcactagtcgcggccgatctaataaacgataacgccgggtggcgtgaggcatgtaaaaggttacatcattatcttgttcgccatccggttggtataaatagacgttcatgttggtttttgtttcagttgcaagttggctgcggcgcgcgcagcacctttgcggccatctgcagaattcgcccttgttactcttcagccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaggcctcgcgagatcttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggccgacacccatttgaaaaaagcgaagacgaaatgttggacttgaatagcatgaagcgtcgtcagcaatacaatgacattggcgttttaaaacacgttcgtaacgttaacgctcgtgactttgtgtactgcctaacaagatacaacatagattgtagactcacaaattacaaacaaattataaaacatgagtttttgtcgtaaaaatgccacttgttttacgagtagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgat

SEQ ID NO: 77 - DNA, 8771 bp, Artificial Sequence
  20ACGZ3P_pCAP11_ polH-VP23
gctaataacaggcctgctggtaatcgcaggcctttttatttggtgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgtagaaggaaggccgtcaaggcctaggcgcgccgtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgggtaccttaaaggggtcgggtaaggtatcgggttccgataggtctggtggttctgtattccccggtgctgtccggggcaaagtccacaaactgggggtcgttgtagttgtttgtgtactggatctctgggttccacctcttggagttttccttcttgagctcccactccatctccacggtgacctgcccggtgctgtactgggtgatgaagctgctgacgggcacgtccgagaagctggtgatatttccgggcacaggcgtgttcttgatgagcatcatgggcggtgggtgtttgagtccgaatccgcccatggccggagaggggtgaaagtgcgcccccgtctctgggatcttggcccagatgggtccttggaggtacacgtccctctccatccacacgctgccgggcacgatttcctggaggttgtacgtgccggtcgcgggggcagtggtggagctctggttgttggtggccatctgcccgccgacgttgtacgccacgcggttcaccggctgcgtctcgctctcgctggtgatgagcatgttgccctcgaggtacgtggcggtggtgcccgggttcgccggctggctgttgaagatcatagtgttctccagggcataggtgttgctgccctggaggttgttggtcatgccgttcggctgcgggggcacctggtaactcgcgccctcgagctccatcctattggtcgtggcgaaggcgctgacactggcgcggttgaccccggagcccaggttccagccctgggttcggcccatgggccccgggaaccagtttttgtaggtgttggcgtatctcccggccaggttcttgttgaactggactccgccagtgttatttgtgctcacgaagcggtacaagtactggtccaccagcgggttggccagcttgaacaggttctgactgggagcgaagctggagtggaagggcacctcctcaaagttgtaggtaaactcaaagttgttgcccgttctcagcatcttgctgggaaagtactctaggcagaagaagctgctcctctcggtgggattttctgtgttgtcgcggttcagcgtcgcgtaaccgtactgcggcagcgtaaagacctgcggagggaaggccggcaggcatccctcggtcccgttgccgacgacgtagggcagctggtagtcgtcgtccgtaaacacttggacggtggaggtgaggttgttggcgatggtggtggtggagtcctgcaccgtgacctctttgacttgaatgttgaagattttgactctgagggaccggggtctgaagccccagtagttgttgatgagtctttgccagtctcgggggctccagtggctgtggaagcggttaaagtcaaagtacccccagggggtgctgtatccaaagtaggcgttggcgttgcttccgtcgacggagccgcttttgatctctcggtactggtggttgttgtagctgggcagcacccaggttcgggtggacttggtgacgactctgtcccccatccacgtggaatcgcaatgccaatctcccgaggcattgcccactccatcggcaccttggttattgtcgcccaatgggccgccacctcccgcagacattgtatcagctcccaaacttgaggctggttgggctgggatttgcagctgctgggatccgctgggtccagcttcggcgtctgacgaggtggaaggcttggagtcctcttcggtccgagccttctttctttttggaaagtggtcgtctatccgctttccggtaggggccgtcttaacaggaccggtatttataggtttttttattacaaaactgttacgaaaacagtaaaatacttatttatttgcgagatggttatcattttaattatctccatgatctattaatattccggagtactagcagctgatgcatagatcagcaactatatattgatagacatttccagtttgtgatattagtttgtgcgtctcattacatcatgaggccggccgttttacgcgtagaattctacccgtaaagcgagtttagttatgagccatgtgcaaaacatgacatcagcttttatttttataacaaatgacatcatttcttgattgtgttttacacgtagaattctactcgtaaagcgagttcagttttgaaaaacaaatgacatcatctttttgattgtgctttacaagtagaattctacccgtaaatcaagttcggttttgaaaaacaaatgagtcatattgtatgatatcatattgcaaaacaaatgactcatcaatcgatcgtgcgttacacgtagaattctactcgtaaagcgagtttatgagccgtgtgcaaaacatgacatcatctcgatttgaaaaacaaatgacatcatccactgatcgtgcattacaagtagaattctactcgtaaagccagttcggttatgagccgtgtacaaaacatgacatcagattatgactcatacttgattgtgttttacgcgtagaattctactcgtaaagcggaccgcatgcaggaccgctttacgagtagaattttacttgtaaaacacaatcaagaaatgatgtcatttttgtacgtgattataaacatgtttaaacatggtacattgaacttaatttttgcaagttgataaacatgattaatgtacgactcatttgtttgtgcaagttgataaacgtgattaatatatgactcatatgtttgtgcaaaaatgatgtcatcgtacaaactcgctttacgagtagaattctacttgtaacgcatgatcaagggatgatgtcatttgtttttttaaaattcaactcgctttacgagtagaattctacttgtaaaacacaatcgagggatgatgtcatttgtagaatgatgtcatttgtttttcaaaaccgaactcgctttacgagtagaattctacttgtaacgcaagatcggtggatgatgtcattttaaaaatgatgtcatcgtacaaactcgctttacgagtagaattctacgtgtaaaactctagatacggacctttaattcaacccaacacaatatattatagttaaataagaattattatcaaatcatttgtatattaattaaaatactatactgtaaattacattttatttacaatcactcgacgaagacttgatcacccctagattcgaaagcggcccgctcactagtaacggccgccagtgtgctggaattcgcccttcgcggatattccggattattcataccgtcccaccatcgggcgcggatcgtaccgggcccaagcttcctgttaagacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacagctcccacaggcaaaagaatcgatgatcatttccccaagcggaaaaaagccagaacagaggaagatagcaaacccagcacaagctccgatgctgaggcaggtccatctggttcacaacaactccagattcctgctcagcctgctagctctctcggcgccgacaccttaagcgctggcggcggaggacccctcggagataacaatcagggcgctgacggcgtcggaaacgctagcggcgactggcactgtgacagcacatggatgggcgatagggtggtgacaaaaagcacaaggacatgggtcctcccttcttataataatcatcaatatagagaaattaagtccggaagcgtggatggctctaatgctaatgcttatttcggctattccacaccttggggatatttcgatttcaatagatttcattcccattggtcccctagggattggcagaggctgattaataattactggggatttaggcctagaagcctgagggtgaagatttttaatatccaggtgaaggaagtgaccgtccaagatagcacaacaacaattgctaataatctgacatcaacagtgcaggtcttcacagatgatgattatcaactcccttatgtggtgggaaatggcacagaaggctgtctccccgcttttccacctcaagtgttcacactccctcaatatggatatgccacactcaatagagataataccgagaaccctacagaaagatcctcctttttttgtttggaatatttcccttccaaaatgctcaggacaggaaataatttcgaattcacatataatttcgaagaagtcccttttcatagctcctttgccccttcccaaaatctctttaaactcgctaatcctctcgtcgatcaatatctctataggtttgtctccaccaacaataccggaggcgccggtggtggtggatcgggaggaggaggttcgggaggtggaggttctggaggcggtggcagcggaggcggcggttcagaggtacaattggtagaatcaggaggaaaattggtgcaggcaggaggctctctgaggctgtcgtgcagcgccagcggtcgcaccttttcgtttagtacctctacgatgggatggttcaggcaggcccctggtaaagaactcgagtttgtagccgcaattgactggtcggatttcaacacctattacgctgatagcgtaaagggaagatttacaatctcaaggcataacccgaggaattcagtgtatctccaactcaacagccttaaaccggaggacacggccgtctattactgcgctgctcatagtgaaacgcgcggtggaactcgctactttgataggccatcgttgtataactactggggccagggtacgcaagtgactgtctcatcgggaggcggaggcgccgttcaattcaataagaatctcgctggcaggtatgctaatacatataagaattggtttcctggccccatgggaaggacacaaggatggaatctcggaagcggagtgaatagagcttctgtgtccgcttttgctaccacaaacagaatggaactggaaggagccagctatcaagtccctcctcaacccaatggaatgacaaataatctgcaaggatccaatacatacgctctcgaaaataccatgatttttaattcccaacccgctaatcccggaacaacagctacatatctggaaggaaatatgctgattacatccgaatccgaaacccaacctgtcaatagagtcgcctataatgtgggaggacaaatggctacaaataatcaatccagcacaacagctcctgctacaggaacctataatctgcaagagattgtccctggttccgtctggatggaaagagatgtctatctgcagggccctatttgggctaaaattcccgaaacaggagcccatttccatcctagccctgctatgggaggctttggcctgaagcatcctccacctatgatgctgattaaaaataccccagtccctggcaacattacatcctttagcgatgtccctgtgtcctcctttattacacaatattccacaggccaagtgacagtcgaaatggaatgggaactgaaaaaagagaatagcaaaagatggaatcccgaaattcaatataccaataattataatgatcctcaattcgtcgatttcgctcctgattccacaggagagtataggacaacaaggcccattggcacaagatatctgacaagacctctgtgaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggccgcttggatcccagaattctaggtacctcttaattaactggcctcatgggccttccttctatcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaaataaaacgaaaggcccagtcttccgactgagcctttcgttttattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaa